(19) United States
(12) Patent Application Publication
(10) Pub. No.: US 2023/0112619 A1
(43) Pub. Date: Apr. 13, 2023

(54) METHOD FOR PREPARING ISOQUINOLINONE COMPOUNDS

(71) Applicants: SHANGHAI DESANO CHEMICAL PHARMACEUTICAL CO., LTD., Shanghai (CN); ARIZEST (SHANGHAI) PHARMATECH CO., LTD., Shanghai (CN); SHANGHAI ACEBRIGHT PHARMACEUTICALS GROUP CO., LTD., Shanghai (CN)

(72) Inventors: Ning JIAO, Shanghai (CN); Zhanqun ZHU, Shanghai (CN); Guofeng SUN, Shanghai (CN); Yanbin SONG, Shanghai (CN); He XING, Shanghai (CN); Xinchao WU, Shanghai (CN)

(21) Appl. No.: 17/756,101

(22) PCT Filed: Oct. 16, 2020
(86) PCT No.: PCT/CN2020/121653

§ 371 Date: Oct. 17, 2022

(30) Foreign Application Priority Data
Oct. 18, 2019 (CN) 201910995506.3
Oct. 18, 2019 (CN) 201910995514.8
Oct. 18, 2019 (CN) 201910996278.1

(51) Int. Cl. C07D 217/26

(52) U.S. Cl. CPC C07D 217/26

(57) ABSTRACT

Provided is a method for preparing isoquinolinone compounds. The preparation method has the advantages of a reasonable route, convenient and easy operations, high preparation yield, high purity, and suitability for industrial production.

### FIELD OF THE INVENTION

The present invention relates to the field of medicinal chemistry, in particular, it relates to a method for preparing isoquinolinone compounds.

### BACKGROUND TECHNIQUE

Roxadustat, the chemical name is (4-hydroxy-1-methyl-7-phenoxy-isoquinolinyl-3-carbonyl)-amino]-acetic acid, the molecular formula is C₁₉H₁₆N₂O₅, the molecular weight is 352.11, the CAS number is 808118-40-3, and the chemical structural formula is:

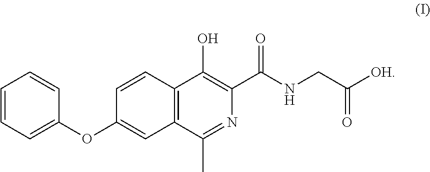 [SMILES: Cc1nc(C(=O)NCC(=O)O)c(O)c2ccc(Oc3ccccc3)cc12]

Roxadustat is a disease developed by FibroGen for the treatment of renal anemia, and was applied for listing in China in November 2017. The drug is the first small-molecule hypoxia-inducible factor prolyl hydroxylase inhibitor (HIF-PHI) drug developed in world for treating renal anemia. The physiological role of hypoxia-inducible factor (HIF) not only increases the expression of erythropoietin, but also increases the expression of erythropoietin receptor and proteins that promote iron absorption and circulation. Roxadustat inhibits PH enzyme by mimicking ketoglutaric acid, one of the substrates of prolyl hydroxylase (PH), and affects the role of PH enzyme in maintaining the rate balance of HIF production and degradation, so as to correct anemia. Roxadustat offers a new treatment option for patients with anemia caused by chronic kidney disease.

However, in the existing Roxadustat preparation technology, the reaction route often needs to react under the conditions of low temperature, high temperature, airtight and pressurized, the reaction conditions are harsh, the processes have high requirements for equipment, the reaction routes are long, and there are many side reactions. These lead to difficulty in subsequent purification, resulting in low yield and low purity of the synthesized Roxadustat. In addition, the existing synthesis method requires expensive catalysts, which is unfavorable for industrialized production.

Therefore, it is necessary to develop a method for synthesizing the isoquinolinone compound Roxadustat with a reasonable route, convenient and easy operations, and suitability for industrial production.

### SUMMARY OF THE INVENTION

The object of the present invention is to provide a preparation method with reasonable route, convenient and easy operations, high yield and high purity, and suitable for industrial production of isoquinolinone compounds.

The first aspect of the present invention provides a method for preparing the compound of formula 3, and the method comprises the following steps:

1) reacting the compound of formula 1 with acyl chloride to obtain the compound of formula 2;

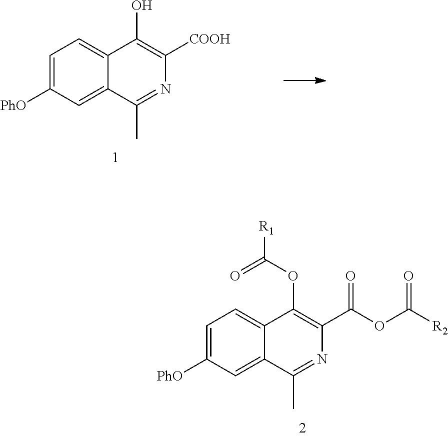

2) after the compound of formula 2 is reacted with aminolysis reagent, the compound of formula 3 is obtained by hydrolysis reaction, wherein the aminolysis reagent is selected from the group consisting of glycine, glycine derivatives, and a combination thereof;

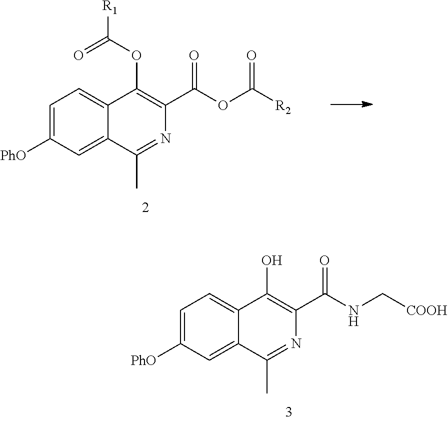

wherein, the acyl chloride is selected from the group consisting of: R₁C(O)Cl, R₂C(O)Cl, and a combination thereof;

R₁ and R₂ are each independently C1-C10 alkyl or C6-C10 aryl.

In another preferred embodiment, R₁ and R₂ are each independently C1-C6 alkyl or C6-C10 aryl.

In another preferred embodiment, in the step 1), the obtained compound of formula 2 is the mixture containing the compound of formula 2 obtained by the reaction.

In another preferred embodiment, in the step 1), the compound of formula 1 is reacted with acyl chloride under the action of a deacid reagent and in a first inert solvent to obtain the compound of formula 2.

In another preferred embodiment, in the step 2), the compound of formula 2 is first aminated by aminolysis reagent, and then hydrolyzed by the first basic reagent to obtain the compound of formula 3.

In another preferred embodiment, in the step 1), the deacid reagent is selected from the group consisting of triethylamine (TEA), 1,8-diazabicycloundec-7-ene (DBU), N,N-diisopropylethylamine (DIEA), pyridine, N-methylmorpholine, and a combination thereof.

In another preferred embodiment, the first inert solvent in the step 1) is selected from the group consisting of tetrahydrofuran, dichloromethane, toluene, and a combination thereof.

In another preferred embodiment, in the step 1), the acyl chloride is selected from the group consisting of acetyl chloride, trimethylacetyl chloride, benzoyl chloride, and a combination thereof.

In another preferred embodiment, in the step 1), the molar ratio of the acyl chloride to the compound of formula 1 is 1-4:1, preferably 2-3.5:1.

In another preferred embodiment, in the step 2), the first basic reagent is selected from the group consisting of sodium hydroxide, potassium hydroxide, lithium hydroxide, or a combination thereof.

In another preferred embodiment, in the step 2), the glycine derivative includes glycinate (salt) or glycinate (ester).

In another preferred embodiment, R₁ and R₂ are each independently methyl, ethyl, n-propyl, phenyl, benzyl, n-butyl, isobutyl, or tert-butyl.

In another preferred embodiment, in the step 2), the molar ratio of the aminolysis reagent to the compound of formula 1 is 1-3:1.

In another preferred embodiment, in the step 1), the molar ratio of the compound of formula 1 to the deacid reagent is 1:1-6, preferably 1:2-4.

In another preferred embodiment, in the step 2), the molar ratio of the compound of formula 2 to the first basic reagent is 1:1-6, preferably 1:2-3.

In another preferred embodiment, in the step 1), the reaction temperature is 15-40° C., preferably 20-30° C.

In another preferred embodiment, in the step 1), the reaction time is 0.5-24 h, preferably 1-5 h, more preferably 3-5 h.

In another preferred embodiment, in the step 2), the reaction temperature is 15-40° C., preferably 20-30° C.

In another preferred embodiment, in the step 2), the reaction time is 0.5-24 h, preferably 4-8 h.

In another preferred embodiment, in the step 2), the molar ratio of the aminolysis reagent to the compound of formula 2 is 1-3:1.

In another preferred embodiment, the glycine derivative is selected from the group consisting of sodium glycinate, methyl glycinate, and a combination thereof.

In another preferred embodiment, the first basic reagent includes sodium hydroxide.

In another preferred embodiment, the glycinate includes sodium glycinate.

In another preferred embodiment, the glycinate includes methyl glycinate.

In another preferred embodiment, in the step 1), the molar ratio of the deacid reagent to the compound of formula 1 is 1-5:1.

In another preferred embodiment, in the step 1), after the compound of formula 1, the deacid reagent and the first inert solvent are mixed, the temperature is lowered to 0-10° C., acyl chloride is added, the temperature is raised to 20-30° C., and the reaction is carried out to obtain a compound of formula 2.

In another preferred embodiment, in the step 1), after the compound of formula 1, the deacid reagent and the first inert solvent are mixed, the temperature is lowered to 0-10° C., acyl chloride is added, the temperature is raised to 20-30° C., and the reaction is carried out to obtain the mixture containing the compound of formula 2, optionally, the compound of formula 2 is obtained by the post-treatment of the mixture containing the compound of formula 2.

In another preferred embodiment, in the step 1), the compound of formula 2 is cooled to 0-10° C., aminolysis reagent is added, and the temperature is raised to 20-40° C. (preferably 20-30° C.) to carry out the aminolysis reaction (preferably, the reaction time is 1-4 h, preferably 2-3 h); after the completion of the aminolysis reaction is detected, the first basic reagent is added to carry out the hydrolysis reaction (preferably, the reaction time is 1-4 h, preferably 1-3 h) to give the compound 3.

In another preferred embodiment, in the step 1), the compound of formula 2 is cooled to 0-10° C., aminolysis reagent is added, and the temperature is raised to 20-40° C. (preferably 20-30° C.) to carry out the aminolysis reaction (preferably, the reaction time is 1-4 h, preferably 2-3 h); after the completion of the aminolysis reaction is detected, the first basic reagent is added to carry out the hydrolysis reaction (preferably, the reaction time is 1-4 h, preferably 1-3 h); the obtained reaction solution is extracted with dichloromethane and water, the pH of the aqueous phase is adjusted to 2-3 with dilute hydrochloric acid, a solid is precipitated, filtered, and washed with acetone to obtain compound 3.

In another preferred embodiment, the mixture containing the compound of formula 2 obtained in the step 1) can be subjected to the subsequent reaction of step 2) without post-treatment.

In another preferred embodiment, the reaction is carried out under normal atmosphere.

In another preferred embodiment, in the step 1), the reaction is carried out under normal atmosphere.

In another preferred embodiment, in the step 2), the reaction is carried out under normal atmosphere.

In another preferred embodiment, the compound of formula 1 is prepared by the following method:

(a) reacting the compound of formula s1 with halogenated reagent in a second inert solvent to obtain the compound of formula s2;

(b) reacting the compound of formula s2 with methylating reagent in a third inert solvent in the presence of palladium catalyst and the second basic reagent to obtain the compound of formula 1;

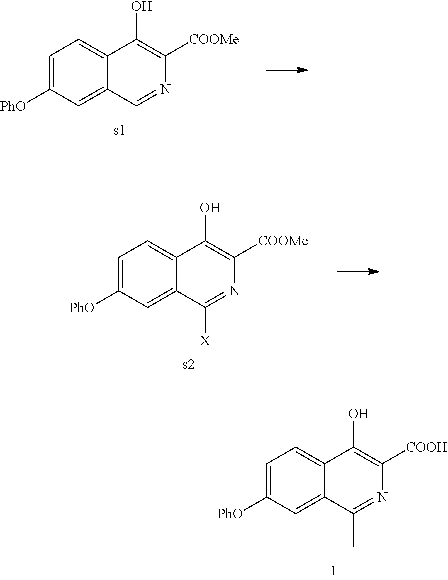

wherein x is Cl, Br or I.

In another preferred embodiment, in the step (a), the reaction is carried out under normal atmosphere.

In another preferred embodiment, in the step (b), the reaction is carried out under normal atmosphere.

In another preferred embodiment, in the step a), the second inert solvent is selected from the group consisting of acetonitrile, methanol, ethanol, ethyl acetate, dichloromethane, and a combination thereof.

In another preferred embodiment, in the step a), the halogenated reagent is selected from the group consisting of NCS, NBS, NIS, dichlorohydantoin, dibromohydantoin, diiodohydantoin, bromine, iodine, and a combination thereof.

In another preferred embodiment, in the step a), the molar ratio of the compound of formula s1 to the halogenated reagent is 1:1-3.

In another preferred embodiment, in the step b), the methylating agent is selected from the group consisting of trimethylboron, methylboronic acid, isopropyl methylborate, potassium methyltrifluoroborate, and a combination thereof.

In another preferred embodiment, in the step b), the second basic reagent is selected from the group consisting of NaOH, KOH, LiOH, Na₂CO₃, K₂CO₃, Na₃PO4, K₃PO4, and a combination thereof.

In another preferred embodiment, in the step b), the palladium catalyst is selected from the group consisting of palladium acetate, bis(triphenylphosphine) palladium dichloride, tetrakis(triphenylphosphine) palladium, tris(benzylideneacetone) dipalladium, bis(diphenylphosphino) ferrocene palladium dichloride, triphenylphosphine palladium dichloride, and a combination thereof.

In another preferred embodiment, in the step b), the third inert solvent includes the mixed solution of ethylene glycol monomethyl ether and water.

In another preferred embodiment, in the step b), the third inert solvent includes the mixed solution of ethylene glycol monomethyl ether and water, and the volume ratio of ethylene glycol monomethyl ether to water is 2-8:1, preferably 2-5:1.

In another preferred embodiment, in the step b), the molar ratio of the compound of formula s2 to the methylating reagent is 1:0.5-4, preferably 1:1-2.

In another preferred embodiment, in the step b), the molar ratio of the compound of formula s2 to the second basic reagent is 1:0.5-5, preferably 1:1-3.

In another preferred embodiment, in the step a), the second inert solvent is acetonitrile, dichloromethane, or a mixed solution of acetonitrile and dichloromethane.

In another preferred embodiment, the volume ratio of acetonitrile to dichloromethane is 1-2:1.

In another preferred embodiment, in the step a), the third inert solvent includes a mixture of ethylene glycol monomethyl ether and water, and the volume ratio of ethylene glycol monomethyl ether to water is.

In another preferred embodiment, in the step a), the compound of formula s1 is added to the second inert solvent, the temperature is cooled to 0-10° C.; after the halogenated reagent is added, the temperature is raised to room temperature to carry out the reaction to obtain the compound of formula s2.

In another preferred embodiment, in the step a), the compound of formula s1 is added to the second inert solvent, the temperature is cooled to 0-10° C.; after the halogenated reagent is added, the temperature is raised to room temperature to carry out the reaction; after the completion of reaction is detected, the mixture is concentrated to dryness, slurried with acetonitrile, and filtered to give the compound of formula s2.

In another preferred embodiment, in the step b), after the compound of formula s2, the palladium catalyst, the second basic reagent, the methylating reagent and the third inert solvent are mixed, the temperature is raised to 90-100° C. to carry out the reaction (preferably, the reaction time is 3-5 h) to obtain the compound of formula 1.

In another preferred embodiment, in the step b), after the compound of formula s2, the palladium catalyst, the second basic reagent, the methylating reagent and the third inert solvent are mixed, the temperature is raised to 90-100° C. to carry out the reaction (preferably, the reaction time is 3-5 h); after the completion of reaction is detected, the temperature is cooled to 20-30° C., then pure water is added, the pH is adjusted to 2-3 by adding hydrochloric acid, the mixture is filtered and washed with methanol to obtain the compound of formula 1.

In another preferred embodiment, in the step a), the halogenated reagent is selected from the group consisting of NBS, NCS, NIS, diiodohydantoin, and a combination thereof.

In another preferred embodiment, in the step a), the molar ratio of compound s1 to the halogenated reagent is 1:1.05-1.3.

In another preferred embodiment, in the step b), the methylating reagent is selected from the group consisting of trimethylboron, methylboronic acid, isopropyl methylboronate, and a combination thereof.

In another preferred embodiment, in the step b), the second basic reagent includes K₃PO4.

In another preferred embodiment, in the step b), the palladium catalyst includes bis(triphenylphosphine) palladium dichloride.

In another preferred embodiment, the reaction does not need to be carried out in closed environment.

In another preferred embodiment, the reaction is carried out under closed or open conditions.

In another preferred embodiment, in the step (a), the reaction temperature is room temperature.

In another preferred embodiment, in the step (a), the reaction time is 0.5-24 h, preferably 2-6 h.

In another preferred embodiment, in the step (b), the reaction temperature is 70-120° C., preferably 90-100° C.

In another preferred embodiment, in the step (b), the reaction time is 0.5-24 h, preferably 2-5 h.

The second aspect of the present invention provides an intermediate of isoquinolinone compounds, and the structure of the intermediate of isoquinolinone compounds is shown in formula 2,

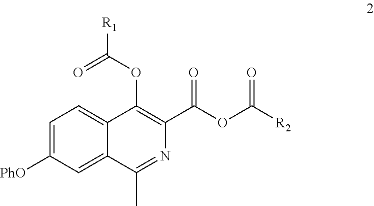

wherein, R₁ and R₂ are each independently C1-C10 alkyl or C6-C10 aryl.

In another preferred embodiment, the intermediate of isoquinolinone compounds is:

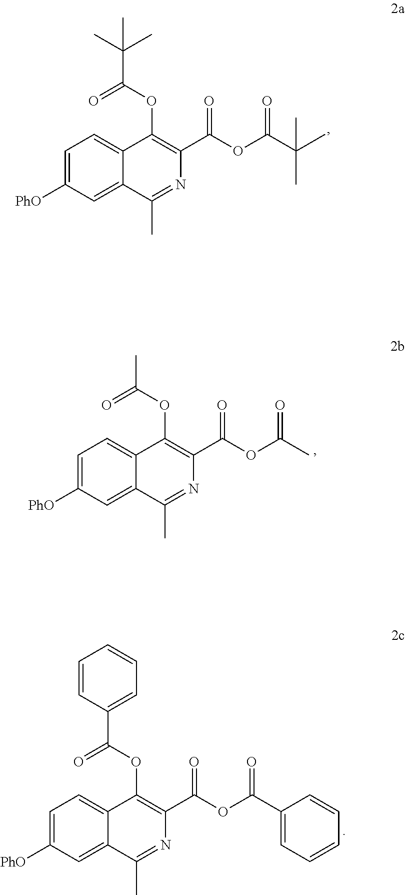 [SMILES: Cc1nc(C(=O)OC(=O)C(C)(C)C)c(OC(=O)C(C)(C)C)c2ccc(Oc3ccccc3)cc12 ; CC(=O)OC(=O)c1nc(C)c2cc(Oc3ccccc3)ccc2c1OC(C)=O ; Cc1nc(C(=O)OC(=O)c2ccccc2)c(OC(=O)c2ccccc2)c2ccc(Oc3ccccc3)cc12]

The third aspect of the present invention provides a method for preparing the intermediate as described in the second aspect of the present invention, and the method comprises the steps of:

1) reacting the compound of formula 1 with acyl chloride to obtain the compound of formula 2;

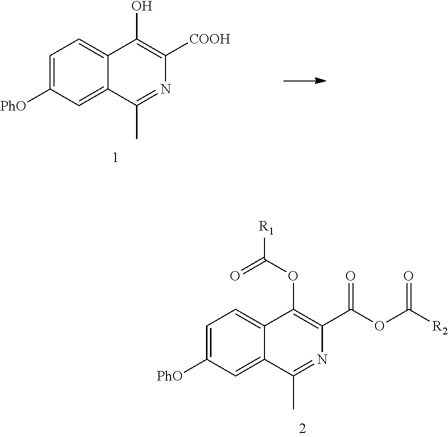

wherein, R₁ and R₂ are as defined in the first aspect of the present invention.

The fourth aspect of the present invention provides a method for preparing the compound of formula 4, and the method comprises the steps of the compound of formula 3 is converted into the compound of formula 4 through an active metal catalysis;

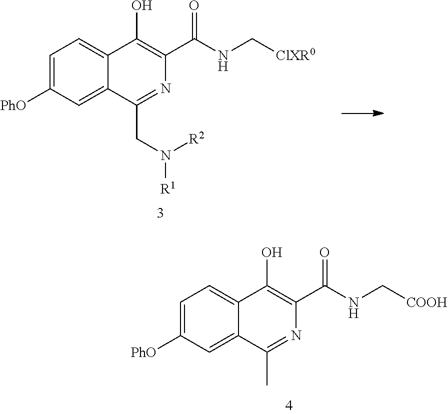

wherein, R⁰ is H, C1-C10 alkyl or C6-C10 aryl; R¹ and R² are independently C1-C10 alkyl, C3-C10 cycloalkyl, C6-C10 aryl, C6-C10 aryl-C1-C10 alkyl-, or R¹ and R² and the nitrogen atom to which they are attached together form 3-10 membered heterocycloalkyl, and the 3-10 membered heterocycloalkyl contains 1-2 (preferably 1 or 2) N atoms and 0-2 (preferably 0, 1 or 2) heteroatoms selected from O and S.

In another preferred embodiment, R⁰ is H or C1-C6 alkyl.

In another preferred embodiment, R⁰ is H or methyl.

In another preferred embodiment, R and R² are independently methyl, benzyl, or R¹ and R² and the nitrogen atom to which they are attached together form piperidinyl or morpholinyl.

In another preferred embodiment, R¹ and R² are independently C1-C6 alkyl, C3-C8 cycloalkyl, C6-C10 aryl, or C6-C10 aryl-C1-C4 alkyl-, or R¹ and R² and the nitrogen atom to which they are attached together form 3-10 membered heterocycloalkyl containing heteroatoms, wherein the heteroatoms include oxygen atom, sulfur atom and/or nitrogen atom.

In another preferred embodiment, when R⁰ is not hydrogen, the method comprises steps 1-2):

1) reacting the compound of formula 3 with an active metal in acid, or an aqueous solution of acid, or an alcoholic solution of acid to obtain the compound of the formula 4′;

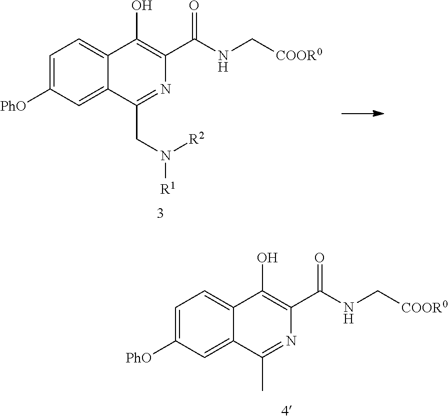

2) reacting the compound of formula 4′ with a base in a first inert solvent to obtain the compound of formula 4;

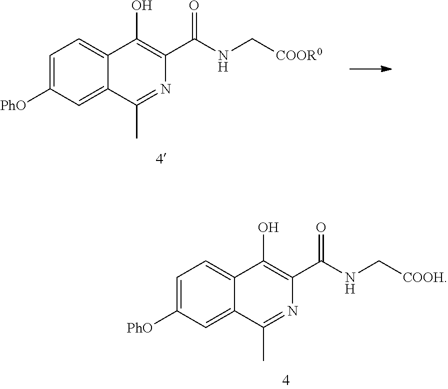

In another preferred embodiment, when R⁰ is not hydrogen, the method comprises steps 1′-2′): 1′) reacting the compound of formula 3 with a base in a second inert solvent to obtain the compound of formula 3′;

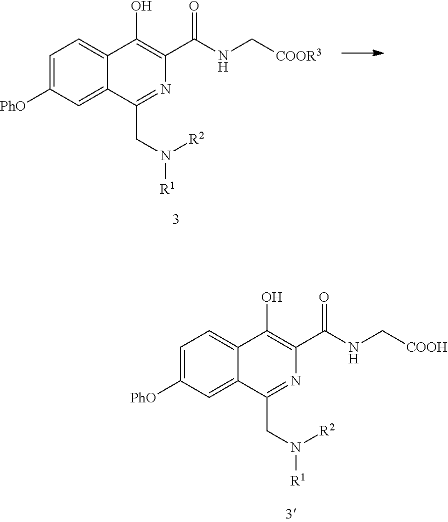

2′) reacting the compound of formula 3′ with an active metal in acid, or an aqueous solution of acid, or an alcoholic solution of acid to obtain the compound of the formula 4;

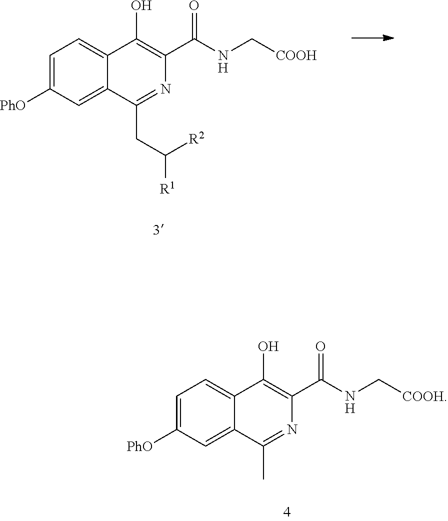

In another preferred embodiment, when R⁰ is hydrogen, the method comprises the following steps: 1″) reacting the compound of formula 3 with an active metal in acid, or an aqueous solution of acid, or an alcoholic solution of acid to obtain the compound of formula 4.

In another preferred embodiment, in the step 1), the step 2′) and/or the step 1″), the acid is inorganic acid or organic acid, wherein the inorganic acid is selected from the group consisting of hydrochloric acid, sulfuric acid, phosphoric acid, and a combination thereof; the organic acid is selected from the group consisting of formic acid, acetic acid, propionic acid, trifluoroacetic acid, trifluoromethanesulfonic acid, and a combination thereof.

In another preferred embodiment, in the step 1), the step 2′) and/or the step 1″), the alcohol solvent of the alcoholic solution of acid is selected from the group consisting of methanol, ethanol, isopropanol, n-butanol, ethylene glycol, and a combination thereof.

In another preferred embodiment, in the step 1), the step 2′) and/or the step 1″), the active metal is selected from the group consisting of magnesium, aluminum, zinc, iron, and a combination thereof.

In another preferred embodiment, in the step 1), the molar ratio of the active metal to the compound of formula 3 is 2-20, preferably 2-10:1.

In another preferred embodiment, in the step 2′), the molar ratio of the active metal to the compound of formula 3′ is 2-20, preferably 5-15.

In another preferred embodiment, in the step 1″), the molar ratio of the active metal to the compound of formula 3 is 2-20, preferably 5-10.

In another preferred embodiment, in the step 1), the reaction temperature is 60-140° C., preferably 60-100° C.

In another preferred embodiment, in the step 1), the reaction time is 0.5-12 h, preferably 4-6 h.

In another preferred embodiment, in the step 2′), the reaction temperature is 60-140° C., preferably 60-100° C.

In another preferred embodiment, in the step 2′), the reaction time is 0.5-12 h, preferably 2-6 h.

In another preferred embodiment, in the step 1″), the reaction temperature is 60-140° C., preferably 70-120° C.

In another preferred embodiment, in the step 2), the first inert solvent is selected from the group consisting of water, methanol, ethanol, isopropanol, n-butanol, ethylene glycol, tetrahydrofuran, 1,4-dioxane, acetonitrile, N,N-dimethylformamide (DMF), N-methylpyrrolidone (NMP), dimethylsulfoxide (DMSO), and a combination thereof.

In another preferred embodiment, in the step 1′), the second inert solvent is selected from water, methanol, ethanol, isopropanol, n-butanol, ethylene glycol, tetrahydrofuran, 1,4-dioxane, acetonitrile, N,N-dimethylformamide (DMF), N-methylpyrrolidone (NMP), dimethylsulfoxide (DMSO), and a combination thereof.

In another preferred embodiment, in the step 2) and/or step 1′), the base is selected from the group consisting of sodium hydroxide, potassium hydroxide, lithium hydroxide, and a combination thereof.

In another preferred embodiment, in the step 2), the reaction time is 2-8 h, preferably 3-6 h.

In another preferred embodiment, in the step 2), the reaction temperature is room temperature.

In another preferred embodiment, in the step 1′), the reaction time is 2-8 h, preferably 3-6 h.

In another preferred embodiment, in the step 1′), the reaction temperature is room temperature.

In another preferred embodiment, the step 1″) comprises the steps: after the compound of formula 3, the active metal, and the acid, or the aqueous solution of acid, or the alcoholic solution of acid are mixed, the mixture is filtered with suction, the filter cake is washed with acetic acid, the filtrate is concentrated, and the mixture is treated with methyl tert-butyl ether to obtain the compound of formula 4.

In another preferred embodiment, the step 1) comprises the steps: the compound of formula 3, the active metal, and the acid, or the aqueous solution of acid, or the alcoholic solution of acid are mixed and reacted; after the reaction is completed, the mixture is filtered with suction, the filter cake is washed with acetic acid, the filtrate is concentrated, and the mixture is treated with methyl tert-butyl ether to obtain the compound of formula 4.

In another preferred embodiment, in the step 2), the compound of formula 4′, the base and the first inert solvent are mixed and reacted; after the reaction is completed, the pH is adjusted to be acidic with acid to precipitate a solid, and the mixture is filtered to obtain the compound of formula 4.

In another preferred embodiment, in the step 1′), the compound of formula 3, the base and the second inert solvent are mixed and reacted; after the reaction is completed, the pH is adjusted to be acidic with acid to precipitate a solid, and the mixture is filtered to obtain the compound of formula 3′.

In another preferred embodiment, in the step 2′), the compound of formula 3′, the active metal and the acid or the aqueous solution of acid or the alcoholic solution of acid are mixed and reacted; after the reaction is completed, the mixture is filtered with suction, the filter cake is washed with acetic acid, the filtrate is concentrated, and the mixture is treated with methyl tert-butyl ether to obtain the compound of formula 4.

In another preferred embodiment, the reaction is carried out under normal pressure.

In another preferred embodiment, in the step 1), the reaction is carried out under normal atmosphere.

In another preferred embodiment, in the step 2), the reaction is carried out under normal atmosphere.

In another preferred embodiment, in the step 1′), the reaction is carried out under normal atmosphere.

In another preferred embodiment, in the step 2′), the reaction is carried out under normal atmosphere.

In another preferred embodiment, in the step 1″), the reaction is carried out under normal atmosphere.

In another preferred embodiment, in the step 1), the step 2′) or the step 1″), the acid is selected from the group consisting of sulfuric acid, phosphoric acid, trifluoroacetic acid, acetic acid, and a combination thereof.

In another preferred embodiment, in the step 1), the step 2′) or the step 1″), the alcohol solvent of the alcoholic solution of acid is isopropanol.

In another preferred embodiment, in the step 1), the step 2′) or the step 1″), the molar ratio of the active metal to the compound of formula 3 is 5-15.

In another preferred embodiment, in the step 1), the step 2′) or the step 1″), the reaction temperature is 80-130° C.

In another preferred embodiment, in the step 2), the first inert solvent is methanol.

In another preferred embodiment, in the step 1′), the second inert solvent is methanol.

In another preferred embodiment, in the step 2), the base is sodium hydroxide.

In another preferred embodiment, in the step 1′), the base is sodium hydroxide.

In another preferred embodiment, the compound of formula 3 is prepared by the following method:

a) reacting the compound of formula 1 with glycine or glycinate to obtain the compound of formula 2;

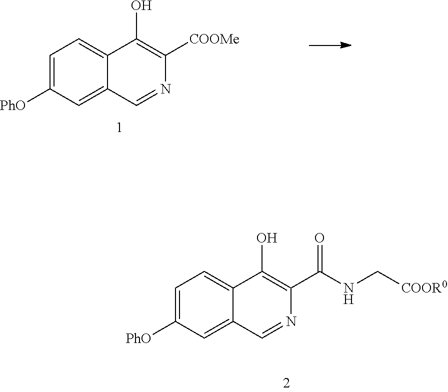

b) reacting the compound of formula 2 with aminal to obtain the compound of formula 3;

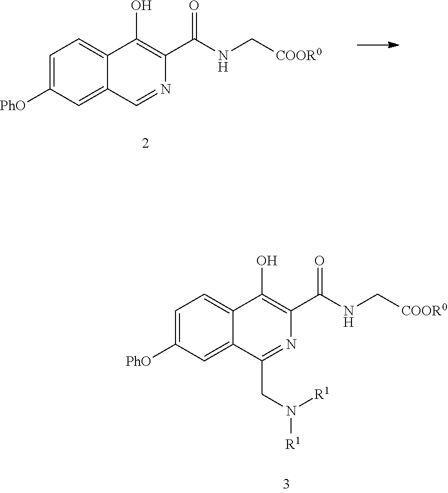

wherein, the glycinate is NH₂—CH₂—C(O)—O—R⁰, and the aminal is (R¹R²)N—CH₂—N(R¹R²); R⁰ is selected from H, C1-C10 alkyl, or C6-C10 aryl; R¹ and R² are independently C1-C10 alkyl, C3-C10 cycloalkyl, C6-C10 aryl, C6-C10 aryl-C1-C10 alkyl-, or R¹ and R² and the nitrogen atom to which they are attached together form 3-10 membered heterocycloalkyl, and the 3-10 membered heterocycloalkyl contains 1-2 (preferably 1 or 2) N atoms and 0-2 (preferably 0, 1 or 2) heteroatoms selected from O and S.

In another preferred embodiment, in the step a), the compound of formula 1 is reacted with glycine or glycinate in the presence of the third inert solvent and organic base to obtain the compound of formula 2.

In another preferred embodiment, in the step b), the compound of formula 2 is reacted with aminal under acid catalysis in the fourth inert solvent to obtain the compound of formula 3.

In another preferred embodiment, in the step a), the third inert solvent is selected from the group consisting of ethylene glycol monomethyl ether, methanol, ethanol, isopropanol, n-butanol, N,N-dimethylformamide (DMF), N-methylpyrrolidone (NMP), dimethylsulfoxide (DMSO), acetonitrile, tetrahydrofuran, 1,4-dioxane, ethyl acetate, isopropyl acetate, dichloromethane, and a combination thereof.

In another preferred embodiment, in the step a), the organic base is selected from the group consisting of triethylamine (TEA), 1,8-diazabicycloundec-7-ene (DBU), N,N-diisopropylethylamine (DIEA), N-methylmorpholine, pyridine, and a combination thereof.

In another preferred embodiment, in the step a), the glycinate is selected from the group consisting of methyl glycinate, ethyl glycinate, benzyl glycinate, and a combination thereof.

In another preferred embodiment, in the step a), the molar ratio of the glycine or glycinate to the compound of formula 1 is 1-4.

In another preferred embodiment, in the step a), the molar ratio of the organic base to the compound of formula 1 is 1-5.

In another preferred embodiment, in the step a), the reaction temperature is 50° C.-100° C.

In another preferred embodiment, in the step a), the reaction time is 4-10 h.

In another preferred embodiment, in the step b), the fourth inert solvent is selected from the group consisting of water, methanol, ethanol, isopropanol, n-butanol, ethylene glycol, 1,4-dioxane, acetic acid, and a combination thereof.

In another preferred embodiment, in the step b), the acid is selected from the group consisting of formic acid, acetic acid, propionic acid, trifluoroacetic acid, trifluoromethanesulfonic acid, hydrochloric acid, sulfuric acid, phosphoric acid, and a combination thereof.

In another preferred embodiment, in the step b), the aminal is selected from the group consisting of tetramethylmethanediamine, tetrabenzylmethanediamine, bispiperazinylmethane, bismorpholinomethane, dipiperidinylmethane, and a combination thereof.

In another preferred embodiment, in the step b), the molar ratio of the aminal to the compound of formula 2 is 1-10.

In another preferred embodiment, in the step b), the reaction temperature is 70° C.-120° C.

In another preferred embodiment, in the step b), the reaction time is 3-10 h.

In another preferred embodiment, in the step a), after the compound of formula 1, the organic base, glycine or glycinate and the third inert solvent are mixed and reacted, the mixture is filtered, the filtrate is diluted with water, the pH is adjusted to be acidic, and crystallization is performed to obtain the compound of formula 2.

In another preferred embodiment, in the step b), after the compound of formula 2, the acid, the aminal and the fourth inert solvent are mixed and reacted, the reaction solution is extracted with water and ethyl acetate to obtain the compound of formula 3.

In another preferred embodiment, in the step a), the third inert solvent is acetonitrile.

In another preferred embodiment, in the step a), the organic base is 1,8-diazabicycloundec-7-ene (DBU).

In another preferred embodiment, in the step a), the glycinate is methyl glycinate.

In another preferred embodiment, in the step a), the molar ratio of the glycine or glycinate to the compound of formula 1 is 1.3-2.5.

In another preferred embodiment, in the step a), the molar ratio of the organic base to the compound of formula 1 is 2-4.

In another preferred embodiment, in the step a), the reaction temperature is 65° C.-90° C.

In another preferred embodiment, in the step b), the fourth inert solvent is selected from the group consisting of water, acetic acid, and a combination thereof.

In another preferred embodiment, in the step b), the acid is selected from the group consisting of trifluoroacetic acid, sulfuric acid, phosphoric acid, and a combination thereof.

In another preferred embodiment, in the step b), the aminal is tetramethylmethanediamine.

In another preferred embodiment, in the step b), the molar ratio of the aminal to the compound of formula 2 is 1.5-5.

In another preferred embodiment, in the step b), the reaction temperature is 80° C.-110° C.

In another preferred embodiment, the reaction is carried out under normal atmosphere.

In another preferred embodiment, in the step b), the reaction is carried out under normal atmosphere.

In another preferred embodiment, in the step a), the reaction is carried out under normal atmosphere.

In another preferred embodiment, the reaction is carried out under normal atmosphere

In another preferred embodiment, the mixture containing the compound of formula 3 obtained in the step b) can be subjected to the subsequent reaction according to the step 1), step 1′) or step 1″) without post-treatment.

In another preferred embodiment, the reaction does not need to be carried out in closed environment.

In another preferred embodiment, the reaction is carried out under closed and open conditions.

The fifth aspect of the present invention provides an intermediate of isoquinolinone compound, and the structure of the intermediate of isoquinolinone compound is shown in formula 3,

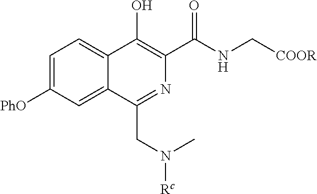

wherein, R⁰ is H, C1-C10 alkyl, or C6-C10 aryl;

R¹ and R² are independently C1-C10 alkyl, C3-C10 cycloalkyl, C6-C10 aryl, C6-C10 aryl-C1-C10 alkyl-, or R¹ and R² and the nitrogen atom to which they are attached together form 3-10 membered heterocycloalkyl, and the 3-10 membered heterocycloalkyl contains 1-2 (preferably 1 or 2) N atoms and 0-2 (preferably 0, 1 or 2) heteroatoms selected from O and S.

In another preferred embodiment, the intermediate is

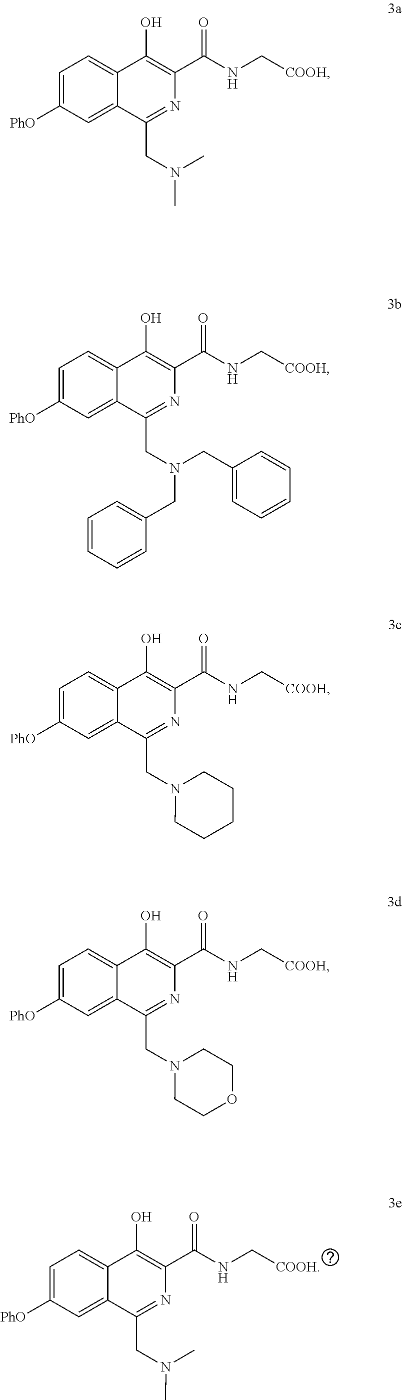 [SMILES: CN(C)Cc1nc(C(=O)NCC(=O)O)c(O)c2ccc(Oc3ccccc3)cc12 ; O=C(O)CNC(=O)c1nc(CN(Cc2ccccc2)Cc2ccccc2)c2cc(Oc3ccccc3)ccc2c1O ; O=C(O)CNC(=O)c1nc(CN2CCCCC2)c2cc(Oc3ccccc3)ccc2c1O ; O=C(O)CNC(=O)c1nc(CN2CCOCC2)c2cc(Oc3ccccc3)ccc2c1O ; CN(C)Cc1nc(C(=O)NCC(=O)O)c(O)c2ccc(Oc3ccccc3)cc12]

The sixth aspect of the present invention provides a method for preparing the compound of formula 3, and the method comprises step a) or step b):

step a):

reacting the compound of formula 1 with glycine to obtain the compound of formula 2, then reacting the compound of formula 2 with alcohol and acyl chloride to obtain the compound of formula 3;

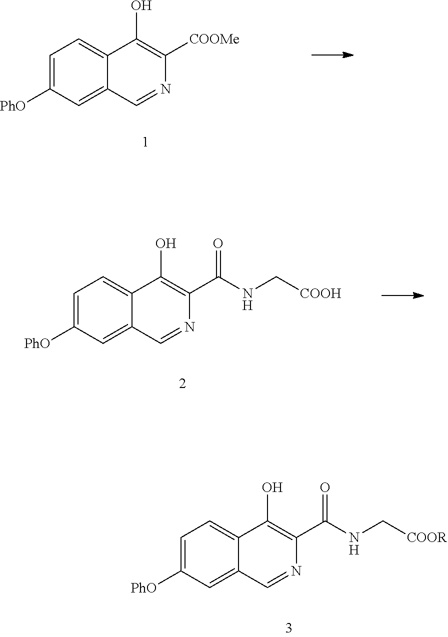

or step b):
reacting the compound of formula 1 with glycinate to obtain the compound of formula 3;

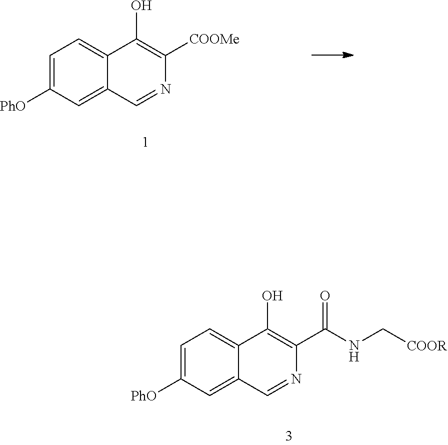

wherein, the acyl chloride is RC(O)Cl, and the glycinate is NH₂—CH₂—C(O)—O—R;

R is C1-C10 alkyl, C6-C10 aryl, C6-C10 aryl-C1-C4 alkyl-, or —R¹OR², wherein R¹ and R² are each independently C1-C10 alkyl.

In another preferred embodiment, R is C1-C6 alkyl, C6-C10 aryl, C6-C10 aryl-C1-C4 alkyl-.

In another preferred embodiment, the step a) comprises the following steps:

a1) reacting the compound of formula 1 with glycine in the first inert solvent under the action of the first basic reagent to obtain the compound of formula 2;

a2) reacting the compound of formula 2 with alcohol and acyl chloride to obtain the compound of formula 3.

In another preferred embodiment, the step b) comprises the following steps:

reacting the compound of formula 1 with glycinate in the second inert solvent under the action of the second basic reagent to obtain the compound of formula 3.

In another preferred embodiment, R is methyl, methoxymethyl, ethyl, ethoxyethyl, propyl, isopropyl, n-butyl, isobutyl, tert-butyl, benzyl or phenyl.

In another preferred embodiment, in the step a1), the first inert solvent is selected from the group consisting of ethylene glycol monomethyl ether, methanol, ethanol, isopropanol, n-butanol, N,N-dimethylformamide (DMF), N-methylpyrrolidone (NMP), dimethylsulfoxide (DMSO), acetonitrile, tetrahydrofuran, 1,4-dioxane, ethyl acetate, isopropyl acetate, dichloromethane, and a combination thereof.

In another preferred embodiment, in the step a1), the first basic reagent is selected from the group consisting of triethylamine (TEA), 1,8-diazabicycloundec-7-ene (DBU), N,N-diisopropylethylamine (DIEA), N-methylmorpholine, pyridine, and a combination thereof.

In another preferred embodiment, in the step a1), the molar ratio of the glycine to the compound of formula 1 is 1-4:1.

In another preferred embodiment, in the step a1), the reaction temperature is 50-100° C.

In another preferred embodiment, in the step a1), the reaction time is 2-12 h, preferably 4-8 h.

In another preferred embodiment, in the step a2), the alcohol is selected from the group consisting of methanol, ethanol, isopropanol, n-butanol, and a combination thereof.

In another preferred embodiment, in the step a2), the acyl chloride is selected from the group consisting of thionyl chloride, acetyl chloride, benzoyl chloride, oxalyl chloride, and a combination thereof.

In another preferred embodiment, in the step a2), the volume ratio of the alcohol to the compound of formula 2 is 1:1-30:1.

In another preferred embodiment, in the step a2), the molar ratio of the acyl chloride to the compound of formula 2 is 1-10:1, preferably 1-6:1.

In another preferred embodiment, in the step a2), the temperatures of all the reactions are the temperature such that the reactions are carried out under reflux conditions.

In another preferred embodiment, in the step a2), the time of all the reactions is 2-8 h, preferably 2-5 h.

In another preferred embodiment, in the step b), the second inert solvent is selected from the group consisting of ethylene glycol monomethyl ether, methanol, ethanol, isopropanol, n-butanol, N,N-dimethylformamide (DMF), N-methylpyrrolidone (NMP), dimethylsulfoxide (DMSO), acetonitrile, tetrahydrofuran, 1,4-dioxane, ethyl acetate, isopropyl acetate, dichloromethane, and a combination thereof.

In another preferred embodiment, in the step b), the second basic reagent is selected from the group consisting of triethylamine (TEA), 1,8-diazabicycloundec-7-ene (DBU), N,N-diisopropylethylamine (DIEA), N-methylmorpholine, pyridine, and a combination thereof.

In another preferred embodiment, in the step b), the glycinate is selected from the group consisting of methyl glycinate, ethyl glycinate, benzyl glycinate, methoxymethyl glycinate, and a combination thereof.

In another preferred embodiment, in the step b), the molar ratio of the glycinate to the compound of formula 1 is 1-4:1.

In another preferred embodiment, in the step b), the reaction temperature is 50-100° C., preferably 55-75° C.

In another preferred embodiment, in the step b), the reaction time is 2-12 h, preferably 4-8 h.

In another preferred embodiment, in the step a1), the reaction time is 5-8 h.

In another preferred embodiment, in the step a1), the molar ratio of the first basic reagent to the compound of formula 1 is 1-6:1, preferably 1-4, more preferably 1.5-4, most preferably 1.5-2.5.

In another preferred embodiment, the step a1) comprises that, after the compound of formula 1, the first basic reagent, glycine and the first inert solvent are mixed and reacted, the mixture is filtered, the pH of the filtrate is adjusted to be acidic, crystallization is performed, and the mixture is filtered to obtain the compound of formula 2.

In another preferred embodiment, the step a2) comprises that, after the compound of formula 2 is mixed with alcohol, the temperature is cooled to 5-15° C., acyl chloride is added, and the mixture is heated to reflux and the reaction is carried out to obtain the compound of formula 3.

In another preferred embodiment, the step a2) comprises that, after the compound of formula 2 is mixed with alcohol, the temperature is cooled to 5-15° C., acyl chloride is added, the mixture is heated to reflux and the reaction is carried out; the reaction is completed, the reaction solution is concentrated to dryness, and dichloromethane is added to extract, the mixture is washed with water, dried and filtered; after the mixture is concentrated and dissolved, petroleum ether is added for crystallization, and the mixture is filtered to obtain the compound of formula 3.

In another preferred embodiment, in the step b), the molar ratio of the second basic reagent to the compound of formula 1 is 1-6:1, preferably 2-4:1.

In another preferred embodiment, the step b) comprises that, after the compound of formula 1, the second basic reagent, glycinate and the second inert solvent are mixed, the reaction is completed, water and ethyl acetate are added for extraction to obtain the compound of formula 3.

In another preferred embodiment, in the step a2), all the reactions are carried out under reflux conditions.

In another preferred embodiment, the reaction is carried out under normal atmosphere.

In another preferred embodiment, in the step a1), the reaction is carried out under normal atmosphere.

In another preferred embodiment, in the step a2), the reaction is carried out under normal atmosphere.

In another preferred embodiment, in the step b), the reaction is carried out under normal atmosphere.

In another preferred embodiment, in the step a1), the first inert solvent is acetonitrile.

In another preferred embodiment, in the step a1), the first basic reagent is 1,8-diazabicycloundec-7-ene (DBU).

In another preferred embodiment, in the step a1), the molar ratio of the glycine to the compound of formula 1 is 1.3-2.5:1, preferably 1-2:1.

In another preferred embodiment, in the step a1), the reaction temperature is 65-90° C.

In another preferred embodiment, in the step a2), the alcohol is methanol.

In another preferred embodiment, in the step a2), the acyl chloride is oxalyl chloride.

In another preferred embodiment, in the step a2), the volume ratio of the alcohol to the compound of formula 2 is 1:1-20:1.

In another preferred embodiment, in the step a2), the molar ratio of the acyl chloride to the compound of formula 2 is 1-4:1, preferably 2-4:1.

In another preferred embodiment, in the step b), the molar ratio of the glycinate to the compound of formula 1 is 1.3-2.5.

In another preferred embodiment, in the step b), the temperature of the reaction in the step b) is 50-75° C.

The seventh aspect of the present invention provides a method for preparing the compound of formula 5, and the method comprises the following steps:
1) reacting the compound of formula 3 with halogenated reagent to obtain the compound of formula 4;

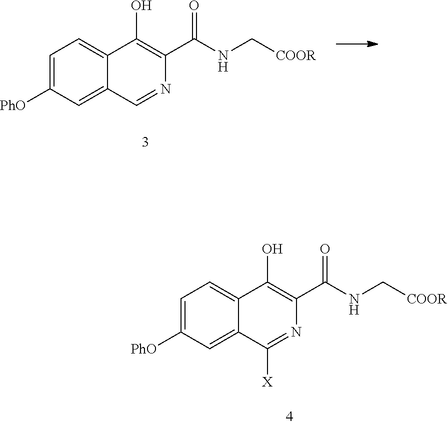

2) reacting the compound of formula 4 with methylating reagent to obtain the compound of formula 5;

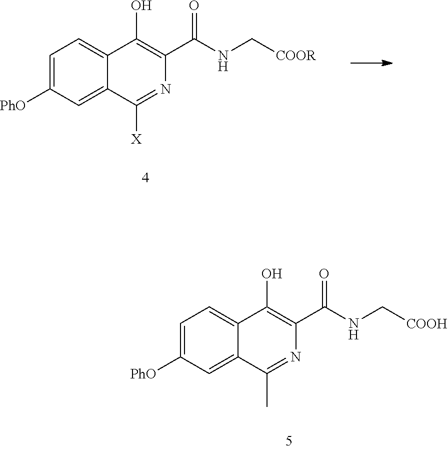

R is C1-C10 alkyl, C6-C10 aryl, C6-C10 aryl-C1-C4 alkyl-, or —R¹OR², wherein R¹ and R² are each independently selected from C1-C10 alkyl, and X is Cl, Br or I.

In another preferred embodiment, R is C1-C6 alkyl, C6-C10 aryl, C6-C10 aryl-C1-C4 alkyl-.

In another preferred embodiment, R is methyl, methoxymethyl, ethyl, ethoxyethyl, propyl, isopropyl, n-butyl, isobutyl, tert-butyl, benzyl or phenyl.

In another preferred embodiment, the compound of formula 3 is prepared by the method described in the sixth aspect of the present invention.

In another preferred embodiment, the halogenated reagent contains halogen X.

In another preferred embodiment, in the step 1), the compound of formula 3 is subjected to halogenation reaction with halogenated reagent in a third inert solvent to obtain the compound of formula 4.

In another preferred embodiment, in the step 2), the compound of formula 4 is reacted with methylating reagent in a fourth inert solvent in the presence of a third basic reagent and palladium catalyst to obtain the compound of formula 5.

In another preferred embodiment, in the compound of formula 3, R is methyl, methoxymethyl, ethyl, ethoxyethyl, propyl, isopropyl, n-butyl, isobutyl, tert-butyl, benzyl or phenyl.

In another preferred embodiment, in the step 1), the third inert solvent is selected from the group consisting of methanol, ethanol, isopropanol, dichloromethane, acetonitrile, tetrahydrofuran, and a combination thereof.

In another preferred embodiment, in the step 1), the halogenated reagent is selected from the group consisting of NCS, dichlorohydantoin, NBS, dibromohydantoin, bromide, tetrabutylammonium tribromide, pyridinium tribromide, iodine, NIS, diiodohydantoin, and a combination thereof.

In another preferred embodiment, in the step 1), the volume ratio of the third inert solvent to the compound of formula 3 is 1:1-30:1.

In another preferred embodiment, in the step 1), the molar ratio of the halogenated reagent to the compound of formula 3 is 1.0-10:1.

In another preferred embodiment, in the step 1), the reaction time is 1-8 h, preferably 1-5 h, more preferably 2-4 h.

In another preferred embodiment, in the step 1), the reaction temperature is 0-30° C., preferably 20-30° C.

In another preferred embodiment, in the step 2), the fourth inert solvent is selected from the group consisting of water, N,N-dimethylformamide, methanol, ethanol, isopropanol, n-butanol, ethylene glycol, ethylene glycol monomethyl ether, ethylene glycol dimethyl ether, 1,4-dioxane, tetrahydrofuran, and a combination thereof.

In another preferred embodiment, in the step 2), the third basic reagent is selected from the group consisting of sodium carbonate, potassium carbonate, potassium acetate, sodium phosphate, potassium phosphate, and a combination thereof.

In another preferred embodiment, in the step 2), the palladium catalyst is selected from the group consisting of bis(triphenylphosphorus) palladium dichloride, palladium acetate, triphenylphosphine palladium acetate, tetrakis(triphenylphosphine) palladium, acetylacetonate palladium, [1,1′-bis(diphenylphosphino)ferrocene] palladium dichloride, [1,1′-bis(diphenylphosphino)ferrocene]palladium dichloride dichloromethane complex, and a combination thereof.

In another preferred embodiment, in the step 2), the methylating reagent is selected from the group consisting of trimethylboron, methylboronic acid, isopropyl methylborate, potassium methyltrifluoroborate, and a combination thereof.

In another preferred embodiment, in the step 2), the volume ratio of the fourth inert solvent to the compound of formula 4 is 1:1-30:1, preferably 20-30:1.

In another preferred embodiment, in the step 2), the molar ratio of the third basic reagent to the compound of formula 4 is 1-10:1, preferably 2-6:1.

In another preferred embodiment, in the step 2), the molar ratio of the methylating agent to the compound of formula 4 is 1-10:1, preferably 2-6:1, more preferably 2-3.5:1.

In another preferred embodiment, in the step 2), the reaction temperature is 50° C.-120° C., preferably 100-120° C.

In another preferred embodiment, in the step 2), the reaction time is 1-10 h, preferably 1-7 h, more preferably 3-5 h.

In another preferred embodiment, in the step 2), the reaction temperature is 80-140° C., preferably 90-130° C., more preferably 100-120° C.

In another preferred embodiment, the step 1) comprises that, after the compound of formula 3 is mixed with the third inert solvent, the temperature is cooled to 0-10° C., halogenated reagent is added, and the reaction is carried out to obtain the compound of formula 4.

In another preferred embodiment, in the step 2), the fourth inert solvent is a mixed solution formed by water and a solvent selected from the group consisting of: ethanol, ethylene glycol monomethyl ether, and a combination thereof.

In another preferred embodiment, in the step 2), the fourth inert solvent is aqueous solution of ethanol, aqueous solution of ethylene glycol monomethyl ether, or aqueous solution of ethanol+ethylene glycol monomethyl ether.

In another preferred embodiment, in the step 2), the fourth inert solvent is aqueous solution of ethanol, and the volume ratio of ethanol to water is 20-40:4-12.

In another preferred embodiment, in the step 2), the fourth inert solvent is aqueous solution of ethanol+ethylene glycol monomethyl ether, and the volume ratio of ethanol, ethylene glycol monomethyl ether and water is 25-35:15-25: 4-12.

In another preferred embodiment, in the step 2), the fourth inert solvent is aqueous solution of ethylene glycol monomethyl ether, and the volume ratio of ethylene glycol monomethyl ether to water is 1-10:1, preferably 2-8:1.

In another preferred embodiment, in the step 2), the molar ratio of the third basic reagent to the compound of formula 4 is 1-3:1.

In another preferred embodiment, in the step 2), the volume ratio of the fourth inert solvent to the compound of formula 4 is 1:1-10:1.

In another preferred embodiment, in the step 2), the molar ratio of the methylating agent to the compound of formula 4 is 1-3.1.

In another preferred embodiment, in the step 2), after the compound of formula 4, the third basic reagent, palladium catalyst, methylating reagent and the fourth inert solvent are mixed and reacted; after the reaction is completed, the reaction solution is filtered, water is added, the pH is adjusted to 3-4 for crystallization, and the mixture is filtered to obtain the compound of formula 5.

In another preferred embodiment, the reaction is carried out under normal atmosphere.

In another preferred embodiment, in the step 1), the reaction is carried out under normal atmosphere.

In another preferred embodiment, in the step 2), the reaction is carried out under normal atmosphere.

In another preferred embodiment, the volume ratio of the dichloromethane and acetonitrile is 0.8-1.2:0.8-1:2.

In another preferred embodiment, in the step 1), the third inert solvent is selected from the group consisting of acetonitrile, dichloromethane, and a combination thereof.

In another preferred embodiment, in the step 1), the halogenated reagent is selected from the group consisting of NBS, NCS, dibromohydantoin, and a combination thereof.

In another preferred embodiment, in the step 1), the volume ratio of the third inert solvent to the compound of formula 3 is 1:1-30:1.

In another preferred embodiment, in the step 1), the molar ratio of the halogenated reagent to the compound of formula 3 is 1-6:1, preferably 1-4:1, more preferably 1-3:1.

In another preferred embodiment, in the step 2), the fourth inert solvent is selected from the group consisting of ethylene glycol monomethyl ether, ethanol, and a combination thereof.

In another preferred embodiment, in the step 2), the third basic reagent is potassium phosphate.

In another preferred embodiment, in the step 2), the palladium catalyst is bis(triphenylphosphorus) palladium dichloride.

In another preferred embodiment, in the step 2), the methylating reagent is methylboronic acid.

In another preferred embodiment, the reaction does not need to be carried out in closed environment.

In another preferred embodiment, the reaction is carried out under closed and open conditions.

The eighth aspect of the present invention provides an intermediate of the isoquinolinone compound, and the structure of the intermediate of the isoquinolinone compound is as shown in formula 3 or formula 4:

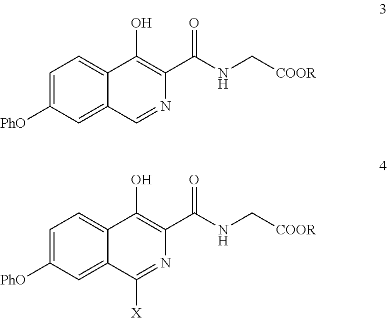

wherein, R is selected from C1-C10 alkyl, C6-C10 aryl, —R¹OR², wherein R¹ and R² are each independently selected from C1-C10 alkyl, and X is Cl, Br or I.

In another preferred embodiment, R is C1-C6 alkyl, C6-C10 aryl, C6-C10 aryl-C1-C4 alkyl-.

In another preferred embodiment, R is methyl, methoxymethyl, ethyl, ethoxyethyl, propyl, isopropyl, n-butyl, isobutyl, tert-butyl, benzyl, or phenyl.

In another preferred embodiment, the isoquinolinone compound intermediate is:

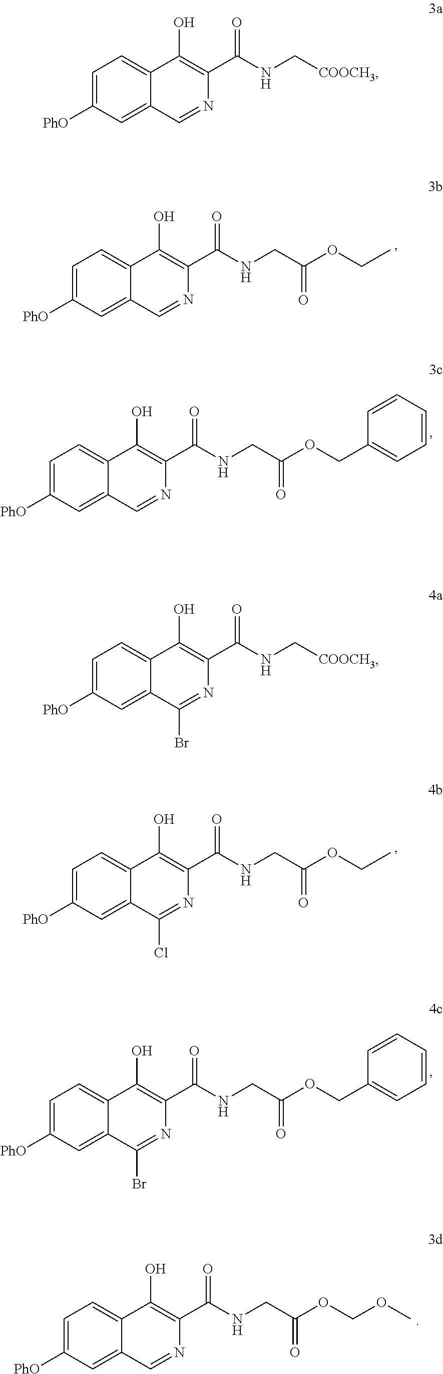

In another preferred embodiment, R is methyl, methoxymethyl, ethyl, ethoxyethyl, propyl, isopropyl, n-butyl, isobutyl, tert-butyl, benzyl or phenyl, and X is Br.

It should be understood that, within the scope of the present invention, the above technical features of the present invention and the technical features specifically described in the following descriptions (such as the examples) can be combined with each other to form a new or preferred technical solution. Due to space limitations, they will not be repeated herein.

### DETAILED DESCRIPTION OF THE INVENTION

After extensive and in-depth research, the present invention unexpectedly found a method for preparing isoquinolinone compounds. The preparation method of the isoquinolinone compounds of the present invention has the advantages of a reasonable route, convenient and easy operations, high preparation yield, high purity, and suitability for industrial production.

On this basis, the inventors have completed the present invention.

Terms

As used herein, the terms “include,” “comprise” and “contain”, which are used interchangeably, include not only closed definitions, but also semi-closed, and open definitions. In other words, the term includes “consist of” and “substantially consist of”.

As used herein, the term “alkyl” refers to a straight (ie, unbranched) or branched chain saturated hydrocarbyl containing only carbon atoms, or a group of a combination of straight and branched chains. When the front of the alkyl group is limited by the number of carbon atoms (e.g., C1-C10 alkyl), it means that the alkyl group has 1 to 10 carbon atoms. Representative examples include, but are not limited to methyl, ethyl, propyl, isopropyl, butyl, isobutyl, sec-butyl, tert-butyl, or the like.

As used herein, the term “cycloalkyl” refers to a ring system group having a saturated or partially saturated monocyclic ring, bicyclic ring or polycyclic ring (fused, bridged or spiro ring). When the front of a certain cycloalkyl group is limited by the number of carbon atoms (e.g., C3-CTO), it means that the cycloalkyl group has 3 to 10 carbon atoms. Representative examples include, but are not limited to cyclopropyl, cyclobutyl, cyclopentyl, cycloheptyl, or the like.

The term “aryl” refers to an aromatic cyclic hydrocarbon group, for example, with 1, or 2 rings, especially refers to a monocyclic and a bicyclic group, such as phenyl, biphenyl or naphthyl. Any aromatic ring having two or more aromatic rings (bicyclic, etc.), the aromatic rings of aryl group may be connected by single bond (such as biphenyl) or fused (such as naphthalene, anthracene, etc.). When the front of an aryl group is limited by the number of carbon atoms, it refers to the number of ring carbon atoms in the aryl group, for example, C6-C10 aryl is an aryl group having 6 to 10 ring carbon atoms. Representative examples include, but are not limited to, phenyl, biphenyl, or naphthyl.

The term “heterocycloalkyl” is also referred to as “heterocyclyl”, which refers to a completely saturated or partially unsaturated cyclic group in which at least one heteroatom is present in a ring having at least one carbon atom. When the front of a heterocycloalkyl group is limited by the number of member, it refers to the ring atom number of heterocycloalkyl group, for example, 3-10 membered heterocycloalkyl refers to a heterocycloalkyl group having 3 to 10 ring atoms. Representative examples include, but are not limited to piperidinyl or morpholinyl. In the present invention, unless otherwise specified, all substituents are unsubstituted substituents.

As used herein, the structures of

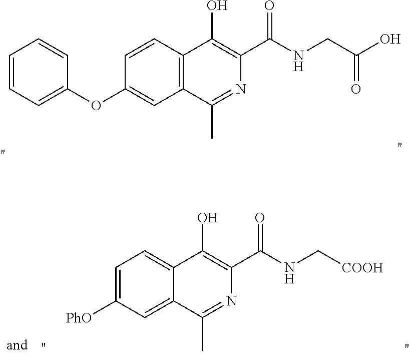 [SMILES: Cc1nc(C(=O)NCC(=O)O)c(O)c2ccc(Oc3ccccc3)cc12 ; Cc1nc(C(=O)NCC(=O)O)c(O)c2ccc(Oc3ccccc3)cc12]

are used interchangeably.

Abbreviations used in the present invention and their meanings are described in the following table:

| Abbreviation | Meaning |
|---|---|
| DMF | N,N-dimethylformamide |
| NMP | N-methylpyrrolidone |
| DMSO | dimethylsulfoxide |
| TEA | triethylamine |
| DBU | 1,8-diazabicycloundec-7-ene |
| DIEA | N,N-diisopropylethylamine |
| DMF | N,N-dimethylformamide |
| TLC | thin layer chromatography |
| DCC | dicyclohexylcarbodiimide |
| EDC | (1-(3-dimethylaminopropyl)-3- |
| | ethylcarbodiimide hydrochloride |
| NCS | N-chlorosuccinimide |
| NBS | N-bromosuccinimide |
| NIS | N-iodosuccinimide |

As used herein, “inert solvent” refers to a solvent that does not react with other substances (eg, starting materials, catalysts, etc.) in the reaction.

As used herein, the structures of

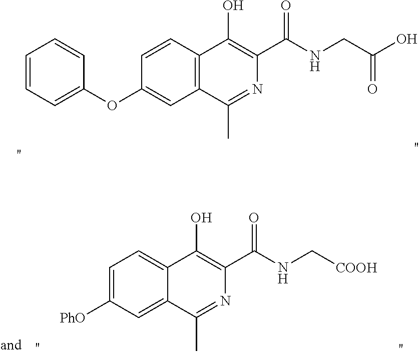 [SMILES: Cc1nc(C(=O)NCC(=O)O)c(O)c2ccc(Oc3ccccc3)cc12 ; Cc1nc(C(=O)NCC(=O)O)c(O)c2ccc(Oc3ccccc3)cc12]

are used interchangeably.

Preparation Method

The present invention provides a preparation method of isoquinolinone compounds of formula 3, as follows in detail:

The present invention provides a method for preparing the compound of formula 3, specifically, the method comprises the following steps:

1) reacting the compound of formula 1 with acyl chloride to obtain the compound of formula 2;

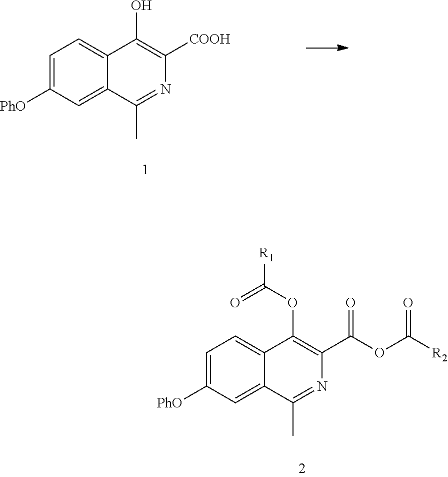

2) after the compound of formula 2 is reacted with aminolysis reagent, the compound of formula 3 is obtained by hydrolysis reaction, wherein the aminolysis reagent is selected from the group consisting of glycine, glycine derivatives, and a combination thereof;

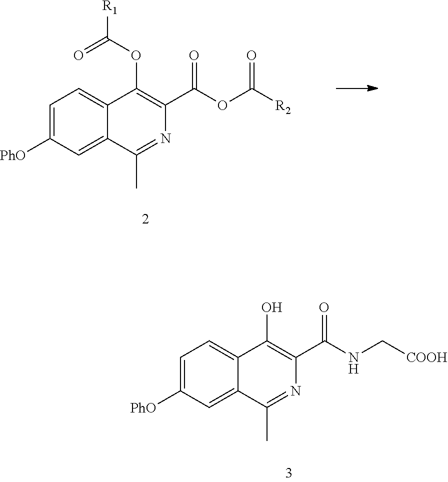

wherein, the acyl chloride is selected from the group consisting of: R₁C(O)Cl, R₂C(O)Cl, and a combination thereof; R₁ and R₂ are each independently C1-C10 alkyl or C6-C10 aryl.

In a preferred embodiment of the present invention, in the step 1), the compound of formula 1 is reacted with acyl chloride under the action of a deacid reagent and in a first inert solvent to obtain the compound of formula 2.

In another preferred embodiment of the present invention, in the step 2), the compound of formula 2 is first aminated by aminolysis reagent, and then hydrolyzed by a first basic reagent to obtain the compound of formula 3.

In another preferred embodiment, in the step 1), the deacid reagent includes (but is not limited to) triethylamine (TEA), 1,8-diazabicycloundec-7-ene (DBU), N,N-diisopropylethylamine (DIEA), pyridine, N-methylmorpholine, and a combination thereof.

In another preferred embodiment, the first inert solvent in the step 1) includes (but is not limited to) tetrahydrofuran, dichloromethane, toluene, and a combination thereof.

In another preferred embodiment, in the step 1), the acyl chloride includes (but is not limited to) acetyl chloride, trimethylacetyl chloride, benzoyl chloride, and a combination thereof.

In another preferred embodiment, in the step 1), the molar ratio of the acyl chloride to the compound of formula 1 is 1-4:1, preferably 2-3.5:1.

In another preferred embodiment, in the step 2), the first basic reagent includes (but is not limited to) sodium hydroxide, potassium hydroxide, lithium hydroxide, and a combination thereof.

In another preferred embodiment, in the step 2), the glycine derivative includes glycinate (salt) or glycinate (ester).

In another preferred embodiment, R₁ and R₂ are each independently methyl, ethyl, n-propyl, phenyl, benzyl, n-butyl, isobutyl, or tert-butyl.

In another preferred embodiment, in the step 2), the molar ratio of the aminolysis reagent to the compound of formula 1 is 1-3:1.

In another preferred embodiment, in the step 1), the molar ratio of the compound of formula 1 to the deacid reagent is 1:1-6, preferably 1:2-4.

In another preferred embodiment, in the step 2), the molar ratio of the compound of formula 2 to the first basic reagent is 1:1-6, preferably 1:2-3.

In another preferred embodiment, in the step 1), the reaction temperature is 15-40° C., preferably 20-30° C.

In another preferred embodiment, in the step 2), the reaction temperature is 15-40° C., preferably 20-30° C.

In another preferred embodiment, the glycine derivative includes (but is not limited to) sodium glycinate, methyl glycinate, and a combination thereof.

In another preferred embodiment, the first basic reagent includes sodium hydroxide.

In another preferred embodiment, the glycinate includes sodium glycinate.

In another preferred embodiment, the glycinate includes methyl glycinate.

In another preferred embodiment, in the step 1), the reaction is carried out under normal atmosphere.

In another preferred embodiment, in the step 2), the reaction is carried out under normal atmosphere.

In another preferred embodiment of the present invention, the compound of formula 1 is prepared by the following method:

(a) reacting the compound of formula s1 with halogenated reagent in a second inert solvent to obtain the compound of formula s2;

(b) reacting the compound of formula s2 with methylating reagent in a third inert solvent in the presence of palladium catalyst and a second basic reagent to obtain the compound of formula 1;

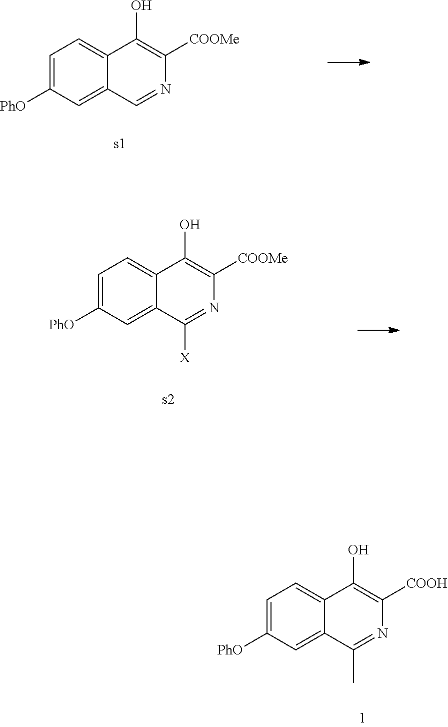

wherein x is Cl, Br or I.

In another preferred embodiment, in the step (a), the reaction is carried out under normal atmosphere.

In another preferred embodiment, in the step (b), the reaction is carried out under normal atmosphere.

In another preferred embodiment, in the step a), the second inert solvent includes (but is not limited to) acetonitrile, methanol, ethanol, ethyl acetate, dichloromethane, and a combination thereof.

In another preferred embodiment, in the step a), the halogenated reagent is selected from the group consisting of NCS, NBS, NIS, dichlorohydantoin, dibromohydantoin, diiodohydantoin, bromine, iodine, and a combination thereof.

In another preferred embodiment, in the step a), the molar ratio of the compound of formula s1 to the halogenated reagent is 1:1-3.

In another preferred embodiment, in the step b), the methylating agent includes (but is not limited to) trimethylboron, methylboronic acid, isopropyl methylborate, potassium methyltrifluoroborate, and a combination thereof.

In another preferred embodiment, in the step b), the second basic reagent includes (but is not limited to) NaOH, KOH, LiOH, Na₂CO₃, K₂CO₃, Na₃PO4, K₃PO4, and a combination thereof.

In another preferred embodiment, in the step b), the palladium catalyst includes (but is not limited to) palladium acetate, bis(triphenylphosphine) palladium dichloride, tetrakis(triphenylphosphine) palladium, tris(benzylideneacetone) dipalladium, bis(diphenylphosphino) ferrocene palladium dichloride, triphenylphosphine palladium dichloride, and a combination thereof.

In another preferred embodiment, in the step (a), the reaction temperature is room temperature.

In another preferred embodiment, in the step (a), the reaction time is 0.5-24 h, preferably 2-6 h.

In another preferred embodiment, in the step (b), the reaction temperature is 70-120° C., preferably 90-100° C.

In another preferred embodiment, in the step (b), the reaction time is 0.5-24 h, preferably 2-5 h.

In the method for preparing the isoquinolinone compound of the formula 3 of the present invention, it has the following excellent technical effects:

1) The present invention unexpectedly adopts the “mixed anhydride method” to construct the amide bond in the chemical structure of isoquinolinone compounds, the compound of formula 1 is reacted with acyl chloride under the action of a deacid reagent to obtain the anhydride of the compound of formula 2, and then aminated with glycine or its derivatives by “one-pot method”, and then base can be used to hydrolyze the 4-position ester group in the molecular structure. Thus, the compound of formula 3 can be easily obtained, and the use of conventional condensing agents such as DCC, EDC and the like is avoided, and the by-products of condensing agents are eliminated in the reaction.

2) In the present invention, since the solvents or reagents used in the process of preparing compound 3 by aminolysis of compound 2 can be the same as those used in preparing compound 2, no post-treatment is required, the “one-pot method” continuous injection from the compound 1 to isoquinolinone compounds can be realized, which greatly shortens the production cycle, improves production efficiency, and is more conducive to industrialized scale-up production. 3) Compared with the prior art, the preparation process of the compound of formula 3 of the present invention has the advantages of simple preparation process, short reaction time, high yield, few by-products, high purity, and has good industrialization prospect.

Intermediate

The present invention also provides an intermediate of the isoquinolinone compounds, and the structure of the intermediate of isoquinolinone compounds is shown in formula 2,

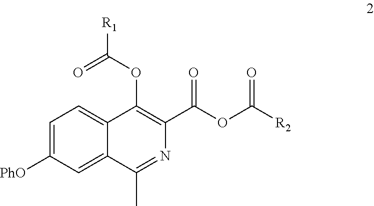

wherein R₁ and R₂ are each independently C1-C10 alkyl or C6-C10 aryl.

In another preferred embodiment, the intermediate of isoquinolinone compounds is:

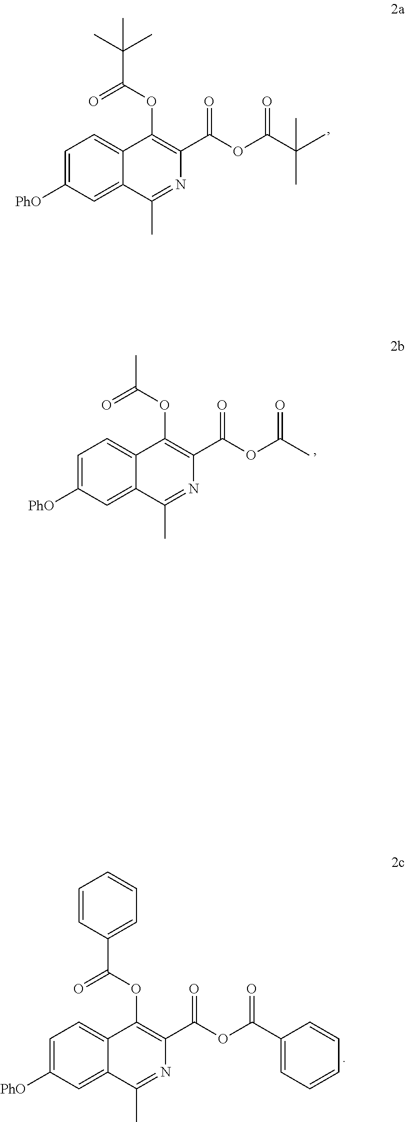 [SMILES: Cc1nc(C(=O)OC(=O)C(C)(C)C)c(OC(=O)C(C)(C)C)c2ccc(Oc3ccccc3)cc12 ; CC(=O)OC(=O)c1nc(C)c2cc(Oc3ccccc3)ccc2c1OC(C)=O ; Cc1nc(C(=O)OC(=O)c2ccccc2)c(OC(=O)c2ccccc2)c2ccc(Oc3ccccc3)cc12]

The present invention also provides a method for preparing the intermediate of isoquinolinone compounds, and the method comprising the steps of:

1) reacting the compound of formula 1 with acyl chloride to obtain the compound of formula 2;

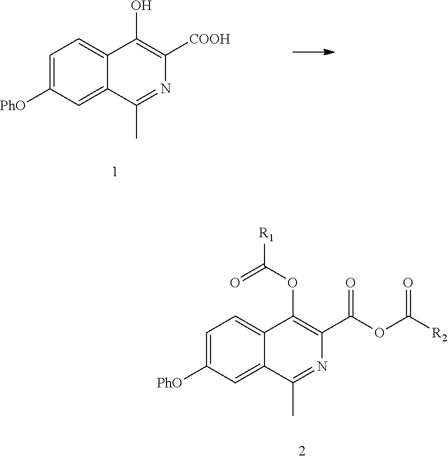

wherein, R₁ and R₂ are defined as above.

Preparation Method of Isoquinolinone Compounds of Formula 4

The present invention provides a preparation method of the compound of formula 3, specifically, the method comprises the following steps:

the compound of formula 3 is converted into the compound of formula 4 through active metal catalysis;

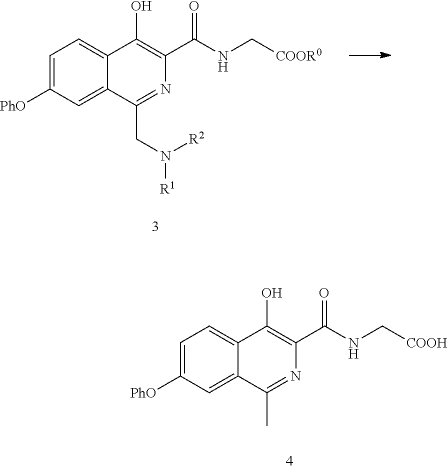

wherein R⁰ is H, C1-C10 alkyl or C6-C10 aryl;

R¹ and R² are independently C1-C10 alkyl, C3-C10 cycloalkyl, C6-C10 aryl, C6-C10 aryl-C1-C10 alkyl-, or R¹ and R² and the nitrogen atom to which they are attached together form 3-10 membered heterocycloalkyl, and the 3-10 membered heterocycloalkyl contains 1-2 (preferably 1 or 2) N atoms and 0-2 (preferably 0, 1 or 2) heteroatoms selected from O and S.

In another preferred embodiment, wherein R⁰ is H or methyl.

In another preferred embodiment, R¹ and R² are independently methyl, benzyl, or R¹ and R² and the nitrogen atom to which they are attached together form piperidinyl or morpholinyl.

In a preferred embodiment of the present invention, when R⁰ is not hydrogen, the method comprises steps 1-2):

1) reacting the compound of formula 3 with an active metal in acid, or an aqueous solution of acid, or an alcoholic solution of acid to obtain the compound of the formula 4′;

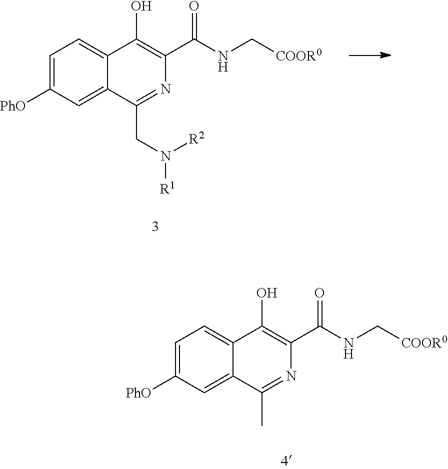

2) reacting the compound of formula 4′ with a base in a first inert solvent to obtain the compound of formula 4;

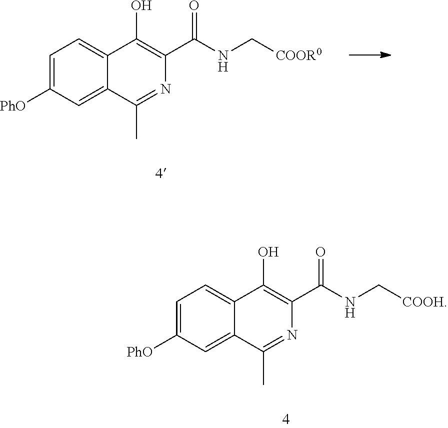

In a preferred embodiment of the present invention, when R⁰ is not hydrogen, the method comprises steps 1′-2′):

1′) reacting the compound of formula 3 with a base in a second inert solvent to obtain the compound of formula 3′;

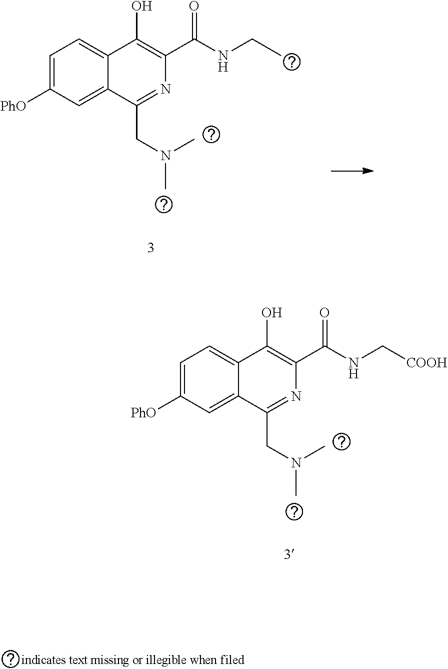 [SMILES: CCNC(=O)c1nc(CN(C)C)c2cc(Oc3ccccc3)ccc2c1O ; CN(C)Cc1nc(C(=O)NCC(=O)O)c(O)c2ccc(Oc3ccccc3)cc12]

2′) reacting the compound of formula 3′ with an active metal in acid, or an aqueous solution of acid, or an alcoholic solution of acid to obtain the compound of the formula 4;

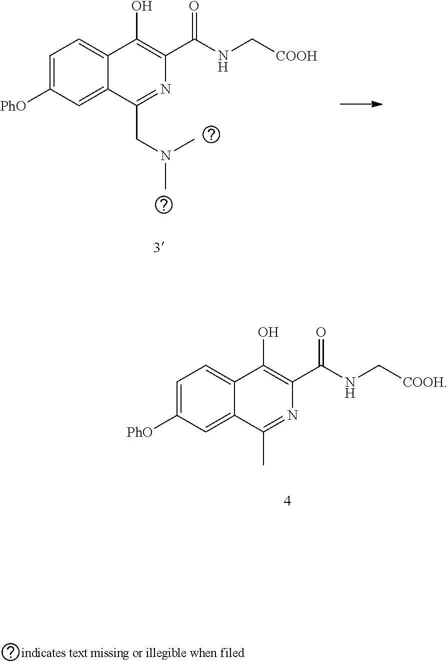 [SMILES: CN(C)Cc1nc(C(=O)NCC(=O)O)c(O)c2ccc(Oc3ccccc3)cc12 ; Cc1nc(C(=O)NCC(=O)O)c(O)c2ccc(Oc3ccccc3)cc12]

In a preferred embodiment of the present invention, when R⁰ is hydrogen, and the method comprises the following steps:

1″) reacting the compound of formula 3 with an active metal in acid, or an aqueous solution of acid, or an alcoholic solution of acid to obtain the compound of formula 4.

In a preferred embodiment of the present invention, in the step 1), the step 2′) and/or the step 1″), the acid is inorganic acid or organic acid, wherein the inorganic acid includes (but is not limited to) hydrochloric acid, sulfuric acid, phosphoric acid, and a combination thereof; the organic acid includes (but is not limited to) formic acid, acetic acid, propionic acid, trifluoroacetic acid, trifluoromethanesulfonic acid, and a combination thereof.

In another preferred embodiment, in the step 1), the step 2′) and/or the step 1″), the alcohol solvent of the alcoholic solution of acid includes (but is not limited to) methanol, ethanol, isopropanol, n-butanol, ethylene glycol, and a combination thereof.

In another preferred embodiment, in the step 1), the step 2′) and/or the step 1″), the active metal includes (but is not limited to) magnesium, aluminum, zinc, iron, and a combination thereof.

In another preferred embodiment, in the step 1), the molar ratio of the active metal to the compound of formula 3 is 2-20, preferably 2-10:1.

In another preferred embodiment, in the step 2′), the molar ratio of the active metal to the compound of formula 3′ is 2-20, preferably 5-15.

In another preferred embodiment, in the step 1″), the molar ratio of the active metal to the compound of formula 3 is 2-20, preferably 5-10.

In another preferred embodiment, in the step 1), the reaction temperature is 60-140° C., preferably 60-100° C.

In another preferred embodiment, in the step 2′), the reaction temperature is 60-140° C., preferably 60-100° C.

In another preferred embodiment, in the step 1″), the reaction temperature is 60-140° C., preferably 70-120° C.

In another preferred embodiment, in the step 2), the first inert solvent includes (but is not limited to) water, methanol, ethanol, isopropanol, n-butanol, ethylene glycol, tetrahydrofuran, 1,4-dioxane, acetonitrile, N,N-dimethylformamide (DMF), N-methylpyrrolidone (NMP), dimethylsulfoxide (DMSO), and a combination thereof.

In another preferred embodiment, in the step 1′), the second inert solvent is selected from water, methanol, ethanol, isopropanol, n-butanol, ethylene glycol, tetrahydrofuran, 1,4-dioxane, acetonitrile, N,N-dimethylformamide (DMF), N-methylpyrrolidone (NMP), dimethylsulfoxide (DMSO), and a combination thereof and/or In the step 2) and/or step 1′), the base includes (but is not limited to) sodium hydroxide, potassium hydroxide, lithium hydroxide, and a combination thereof.

In another preferred embodiment, in the step 2), the reaction temperature is room temperature.

In another preferred embodiment, in the step 1′), the reaction temperature is room temperature.

In another preferred embodiment, in the step 1), the reaction is carried out under normal atmosphere.

In another preferred embodiment, in the step 2), the reaction is carried out under normal atmosphere.

In another preferred embodiment, in the step 1′), the reaction is carried out under normal atmosphere.

In another preferred embodiment, in the step 2′), the reaction is carried out under normal atmosphere.

In another preferred embodiment, in the step 1″), the reaction is carried out under normal atmosphere.

In another preferred embodiment of the present invention, the compound of formula 3 is prepared by the following method:

a) reacting the compound of formula 1 with glycine or glycinate to obtain the compound of formula 2;

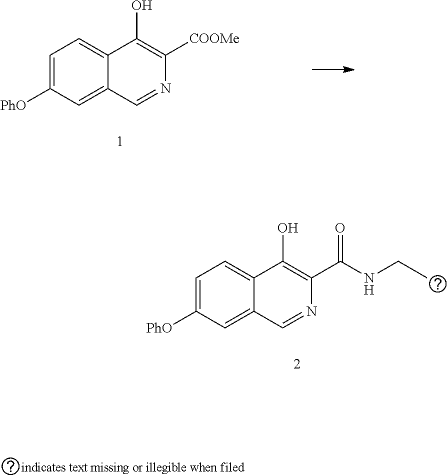 [SMILES: COC(=O)c1ncc2cc(Oc3ccccc3)ccc2c1O ; CCNC(=O)c1ncc2cc(Oc3ccccc3)ccc2c1O]

b) reacting the compound of formula 2 with aminal to obtain the compound of formula 3;

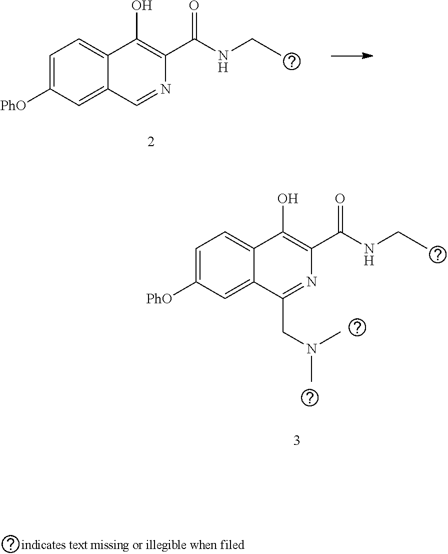

wherein, the glycinate is NH₂—CH₂—C(O)—O—R⁰, and the aminal is (R¹R²)N—CH₂—N(R¹R²);

R⁰ is selected from H, C1-C10 alkyl, or C6-C10 aryl;

R¹ and R² are independently C1-C10 alkyl, C3-C10 cycloalkyl, C6-C10 aryl, C6-C10 aryl-C1-C10 alkyl-, or R¹ and R² and the nitrogen atom to which they are attached together form 3-10 membered heterocycloalkyl, and the 3-10 membered heterocycloalkyl contains 1-2 (preferably 1 or 2) N atoms and 0-2 (preferably 0, 1 or 2) heteroatoms selected from O and S.

In another preferred embodiment, wherein R⁰ is H or C1-C6 alkyl.

In another preferred embodiment, wherein R⁰ is H or methyl.

In another preferred embodiment, R¹ and R² are independently methyl, benzyl, or R and R² and the nitrogen atom to which they are attached together form piperidinyl or morpholinyl.

In another preferred embodiment, R¹ and R² are independently C1-C6 alkyl, C3-C8 cycloalkyl, C6-C10 aryl, or C6-C10 aryl-C1-C4 alkyl-, or R¹ and R² and the nitrogen atom to which there are attached together form 3-10 membered heterocycloalkyl containing heteroatoms, wherein the heteroatoms include oxygen atom, sulfur atom and/or nitrogen atom.

In a preferred embodiment of the present invention, in the step a), the compound of formula 1 is reacted with glycine or glycinate in the presence of a third inert solvent and organic base to obtain the compound of formula 2.

In another preferred embodiment of the present invention, in the step b), the compound of formula 2 is reacted with aminal under acid catalysis in a fourth inert solvent to obtain the compound of formula 3.

In another preferred embodiment, in the step a), the third inert solvent includes (but is not limited to) ethylene glycol monomethyl ether, methanol, ethanol, isopropanol, n-butanol, N,N-dimethylformamide (DMF), N-methylpyrrolidone (NMP), dimethylsulfoxide (DMSO), acetonitrile, tetrahydrofuran, 1,4-dioxane, ethyl acetate, isopropyl acetate, dichloromethane, and a combination thereof.

In another preferred embodiment, in the step a), the organic base includes (but is not limited to) triethylamine (TEA), 1,8-diazabicycloundec-7-ene (DBU), N,N-diisopropylethylamine (DIEA), N-methylmorpholine, pyridine, and a combination thereof.

In another preferred embodiment, in the step a), the glycinate includes (but is not limited to) methyl glycinate, ethyl glycinate, benzyl glycinate, and a combination thereof.

In another preferred embodiment, in the step a), the molar ratio of the glycine or glycinate to the compound of formula 1 is 1-4.

In another preferred embodiment, in the step a), the molar ratio of the organic base to the compound of formula 1 is 1-5.

In another preferred embodiment, in the step a), the reaction temperature is 50° C.-100° C.

In another preferred embodiment, in the step b), the fourth inert solvent includes (but is not limited to) water, methanol, ethanol, isopropanol, n-butanol, ethylene glycol, 1,4-dioxane, acetic acid, and a combination thereof.

In another preferred embodiment, in the step b), the acid includes (but is not limited to) formic acid, acetic acid, propionic acid, trifluoroacetic acid, trifluoromethanesulfonic acid, hydrochloric acid, sulfuric acid, phosphoric acid, and a combination thereof.

In another preferred embodiment, in the step b), the aminal includes (but is not limited to) tetramethylmethanediamine, tetrabenzylmethanediamine, bispiperazinylmethane, bismorpholinomethane, dipiperidinylmethane, and a combination thereof.

In another preferred embodiment, in the step b), the molar ratio of the aminal to the compound of formula 2 is 1-10.

In another preferred embodiment, in the step b), the reaction temperature is 70° C.-120° C.

In the step b), the reaction time is 3-10 h.

In the method for preparing the isoquinolinone compounds of the formula 4 of the present invention, it has the following excellent technical effects:

1) In the present invention, the compound of formula 1 is first reacted with glycine for aminolysis, and glycine group is preferentially introduced on the isoquinoline core. This step can be completed only by heating under normal atmosphere, without in closed environment such as using a sealed tube, which reduces the requirements for equipment in the preparation process, simplifies the operation steps, and reduces potential safety hazards.

2) In the present invention, since the solvents or reagents used in the preparation of the compound of formula 4 can be the same as those used in the preparation of the compound of formula 3, no post-treatment is required, and the “one-pot method” continuous injection from the compound of formula 2 to isoquinolinone compounds can be realized, which greatly shortens the production cycle, improves production efficiency, and is more conducive to industrialized scale-up production.

3) The preparation method of the isoquinolinone compounds of the present invention has a short reaction route, does not need to use precious metals (such as palladium) as catalyst, can not only reduce the content of heavy metals in the final product, which facilitate post-processing and improve product quality, but also reduce the cost of production.

4) Compared with the prior art, the preparation process of the isoquinolinone compounds of the present invention has the advantages of simple preparation process, short reaction time, high yield, few by-products, the results of scale-up production is good, and it has good industrialization prospect.

Intermediates of Isoquinolinone Compounds

The present invention provides an intermediate of isoquinolinone compounds, and the structure of the intermediate of isoquinolinone compounds is shown in formula 3;

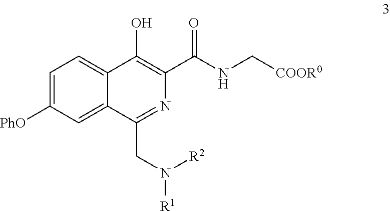

wherein, R⁰ is H, C1-C10 alkyl, or C6-C10 aryl;

R¹ and R² are independently C1-C10 alkyl, C3-C10 cycloalkyl, C6-C10 aryl, C6-C10 aryl-C1-C10 alkyl-, or R¹ and R² and the nitrogen atom to which they are attached together form 3-10 membered heterocycloalkyl, and the 3-10 membered heterocycloalkyl contains 1-2 (preferably 1 or 2) N atoms and 0-2 (preferably 0, 1 or 2) heteroatoms selected from O and S.

In another preferred embodiment, wherein R⁰ is H or C1-C6 alkyl.

In another preferred embodiment, wherein R⁰ is H or methyl.

In another preferred embodiment, R¹ and R² are independently methyl, benzyl, or R¹ and R² and the nitrogen atom to which they are attached together form piperidinyl or morpholinyl.

In another preferred embodiment, R¹ and R² are independently C1-C6 alkyl, C3-C8 cycloalkyl, C6-C10 aryl, or C6-C10 aryl-C1-C4 alkyl-, or R¹ and R² and the nitrogen atom to which they are attached together form 3-10 membered heterocycloalkyl containing heteroatoms, wherein the heteroatoms include oxygen atom, sulfur atom and/or nitrogen atom.

In another preferred embodiment, the intermediate is

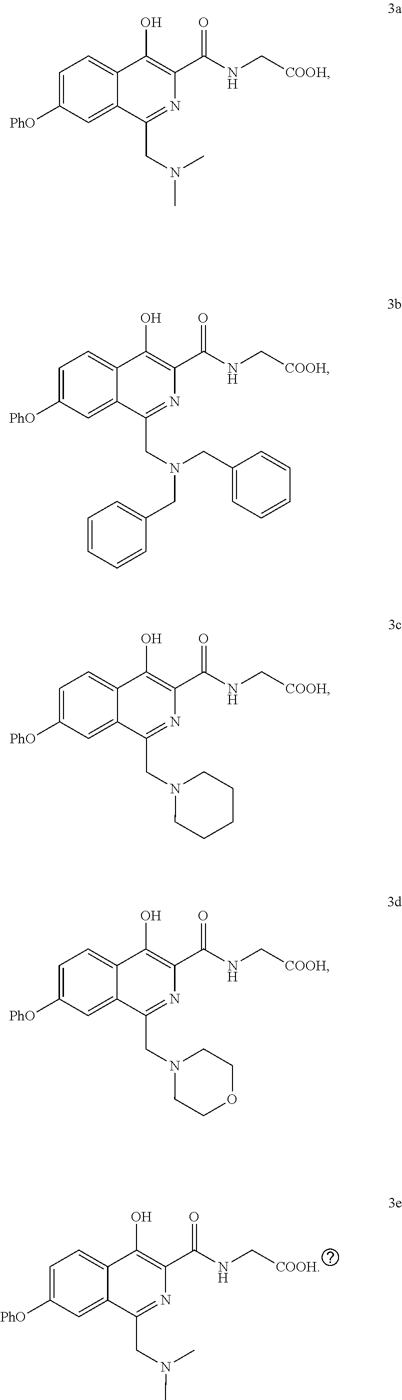 [SMILES: CN(C)Cc1nc(C(=O)NCC(=O)O)c(O)c2ccc(Oc3ccccc3)cc12 ; O=C(O)CNC(=O)c1nc(CN(Cc2ccccc2)Cc2ccccc2)c2cc(Oc3ccccc3)ccc2c1O ; O=C(O)CNC(=O)c1nc(CN2CCCCC2)c2cc(Oc3ccccc3)ccc2c1O ; O=C(O)CNC(=O)c1nc(CN2CCOCC2)c2cc(Oc3ccccc3)ccc2c1O ; CN(C)Cc1nc(C(=O)NCC(=O)O)c(O)c2ccc(Oc3ccccc3)cc12]

The Preparation Method of Isoquinolinone Compounds of Formula 3

The present invention also provides a method for preparing the compound of formula 3, specifically, the present invention provides a method for preparing the compound of formula 3, and the method comprises step a) or step b):

step a):

reacting the compound of formula 1 with glycine to obtain the compound of formula 2, then reacting the compound of formula 2 with alcohol and acyl chloride to obtain the compound of formula 3;

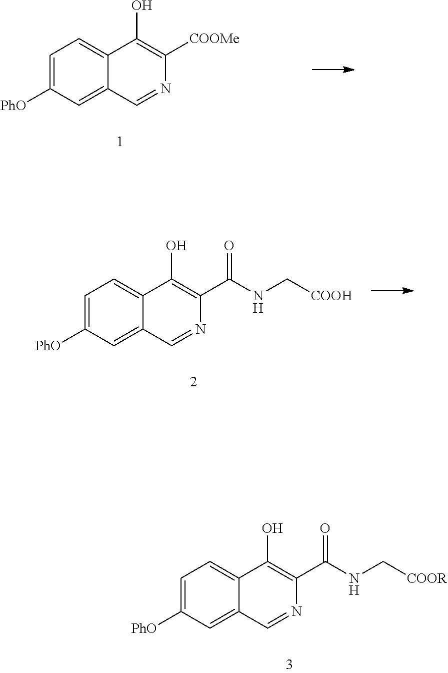

or step b):

reacting the compound of formula 1 with glycinate to obtain the compound of formula 3;

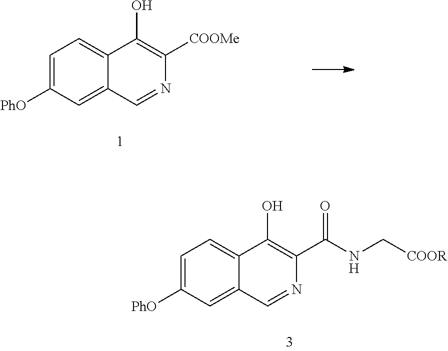

wherein, the acyl chloride is RC(O)Cl, and the glycinate is NH₂—CH₂—C(O)—O—R;

R is C1-C10 alkyl, C6-C10 aryl, C6-C10 aryl-C1-C4 alkyl-, or —R¹OR², wherein R¹ and R² are each independently C1-C10 alkyl.

In another preferred embodiment, R is methyl, methoxymethyl, ethyl, ethoxyethyl, propyl, isopropyl, n-butyl, isobutyl, tert-butyl, benzyl or phenyl.

In a preferred embodiment of the present invention, the step a) comprises the following steps:

a1) reacting the compound of formula 1 with glycine in a first inert solvent under the action of a first basic reagent to obtain the compound of formula 2;

a2) reacting the compound of formula 2 with alcohol and acyl chloride to obtain the compound of formula 3.

In another preferred embodiment of the present invention, the step b) comprises the following steps:

reacting the compound of formula 1 with glycinate in a second inert solvent under the action of a second basic reagent to obtain the compound of formula 3.

In a preferred embodiment, in the step a1), the first inert solvent includes (but is not limited to) ethylene glycol monomethyl ether, methanol, ethanol, isopropanol, n-butanol, N,N-dimethylformamide (DMF), N-methylpyrrolidone (NMP), dimethylsulfoxide (DMSO), acetonitrile, tetrahydrofuran, 1,4-dioxane, ethyl acetate, isopropyl acetate, dichloromethane, and a combination thereof.

In another preferred embodiment, in the step a1), the first basic reagent includes (but is not limited to) triethylamine (TEA), 1,8-diazabicycloundec-7-ene (DBU), N,N-diisopropylethylamine (DIEA), N-methylmorpholine, pyridine, and a combination thereof.

In another preferred embodiment, in the step a1), the molar ratio of the glycine to the compound of formula 1 is 1-4:1.

In another preferred embodiment, in the step a1), the reaction temperature is 50-100° C.

In another preferred embodiment, in the step a2), the alcohol includes (but is not limited to) methanol, ethanol, isopropanol, n-butanol, and a combination thereof.

In another preferred embodiment, in the step a2), the acyl chloride includes (but is not limited to) thionyl chloride, acetyl chloride, benzoyl chloride, oxalyl chloride, and a combination thereof.

In another preferred embodiment, in the step a2), the molar ratio of acyl chloride to the compound of formula 2 is 1-10:1, preferably 1-6:1.

In another preferred embodiment, in the step a2), the temperatures of all the reactions are the temperature such that the reactions are carried out under reflux conditions.

In another preferred embodiment, in the step b), the second inert solvent includes (but is not limited to) ethylene glycol monomethyl ether, methanol, ethanol, isopropanol, n-butanol, N,N-dimethylformamide (DMF), N-methylpyrrolidone (NMP), dimethylsulfoxide (DMSO), acetonitrile, tetrahydrofuran, 1,4-dioxane, ethyl acetate, isopropyl acetate, dichloromethane, and a combination thereof.

In another preferred embodiment, in the step b), the second basic reagent includes (but is not limited to) triethylamine (TEA), 1,8-diazabicycloundec-7-ene (DBU), N,N-diisopropylethylamine (DIEA), N-methylmorpholine, pyridine, and a combination thereof.

In another preferred embodiment, in the step b), the glycinate includes (but is not limited to) methyl glycinate, ethyl glycinate, benzyl glycinate, methoxymethyl glycinate, and a combination thereof.

In another preferred embodiment, in the step b), the molar ratio of glycinate to the compound of formula 1 is 1-4:1.

In another preferred embodiment, in the step b), the reaction temperature is 50-100° C., preferably 55-75° C.

In another preferred embodiment, in the step a1), the molar ratio of the first basic reagent to the compound of formula 1 is 1-6:1, preferably 1-4, more preferably 1.5-4, most preferably 1.5-2.5.

In another preferred embodiment, the reaction is carried out under normal atmosphere.

Preparation Method of the Isoquinolinone Compounds of Formula 5

The present invention provides a method for preparing the compound of formula 5; specifically, the method comprises the following steps:

1) reacting the compound of formula 3 with halogenated reagent to obtain the compound of formula 4;

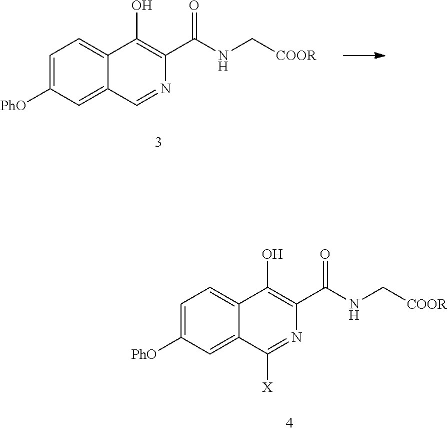

2) reacting the compound of formula 4 with methylating reagent to obtain the compound of formula 5:

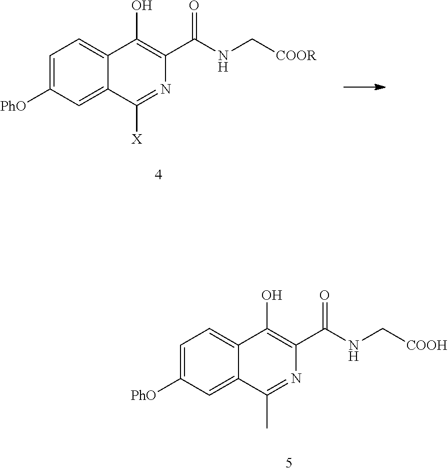

R is C1-C10 alkyl, C6-C10 aryl, C6-C10 aryl-C1-C4 alkyl-, or —R¹OR², wherein R¹ and R² are each independently selected from C1-C10 alkyl, and X is Cl, Br or I.

In a preferred embodiment, the compound of formula 3 is prepared by the method described as above.

In another preferred embodiment, in the step 1), the compound of formula 3 is subjected to halogenation reaction with halogenated reagent in a third inert solvent to obtain the compound of formula 4.

In another preferred embodiment, in the step 2), the compound of formula 4 is reacted with methylating reagent in a fourth inert solvent in the presence of a third basic reagent and palladium catalyst to obtain the compound of formula 5.

In another preferred embodiment, in the step 1), the third inert solvent is selected from the group consisting of methanol, ethanol, isopropanol, dichloromethane, acetonitrile, tetrahydrofuran, and a combination thereof.

In another preferred embodiment, in the step 1), the halogenated reagent is selected from the group consisting of NCS, dichlorohydantoin, NBS, dibromohydantoin, bromide, tetrabutylammonium tribromide, pyridinium tribromide, iodine, NIS, diiodohydantoin, and a combination thereof.

In another preferred embodiment, in the step 1), the molar ratio of halogenated reagent to the compound of formula 3 is 1.0-10:1.

In another preferred embodiment, in the step 1), the reaction temperature is 0-30° C., preferably 20-30° C.

In another preferred embodiment, in the step 2), the fourth inert solvent includes (but is not limited to) water, N,N-dimethylformamide, methanol, ethanol, isopropanol, n-butanol, ethylene glycol, ethylene glycol monomethyl ether, ethylene glycol dimethyl ether, 1,4-dioxane, tetrahydrofuran, and a combination thereof.

In another preferred embodiment, in the step 2), the third basic reagent includes (but is not limited to) sodium carbonate, potassium carbonate, potassium acetate, sodium phosphate, potassium phosphate, and a combination thereof.

In another preferred embodiment, in the step 2), the palladium catalyst includes (but is not limited to) bis(triphenylphosphorus) palladium dichloride, palladium acetate, triphenylphosphine palladium acetate, tetrakis(triphenylphosphine) palladium, acetylacetonate palladium, [1,1′-bis(diphenylphosphino)ferrocene] palladium dichloride, [1,1′-bis(diphenylphosphino)ferrocene]palladium dichloride dichloromethane complex, and a combination thereof.

In another preferred embodiment, in the step 2), the methylating reagent includes (but is not limited to) trimethylboron, methylboronic acid, isopropyl methylborate, potassium methyltrifluoroborate, and a combination thereof.

In another preferred embodiment, in the step 2), the volume ratio of the fourth inert solvent to the compound of formula 4 is 1:1 to 30:1, preferably 20-30:1.

In another preferred embodiment, in the step 2), the molar ratio of methylating agent to the compound of formula 4 is 1-10.1, preferably 2-6:1, more preferably 2-3.5:1.

In another preferred embodiment, in the step 2), the reaction temperature is 50° C.-120° C., preferably 100-120° C.

In another preferred embodiment, in the step 2), the reaction temperature is 80-140° C., preferably 90-130° C., more preferably 100-120° C.

In another preferred embodiment, in the step 1), the reaction is carried out under normal atmosphere.

In another preferred embodiment, in the step 2), the reaction is carried out under normal atmosphere.

In the preparation method of the isoquinolinone compounds of the formula 3 of the present invention, it has the following excellent technical effects:

1) In the present invention, the compound of formula 1 is first reacted with glycine for aminolysis, and the glycine group is preferentially introduced on the isoquinoline core. This step can be completed only by heating under normal atmosphere, without in closed environment such as using a sealed tube, which reduces the requirements for equipment in the preparation process, simplifies the operation steps, and reduces potential safety hazards.

2) In the process of preparing the isoquinolinone compounds of formula 3 from the compound of formula 3 of the present invention, the halogenation reaction and the Suzuki coupling reaction are fast.

3) Compared with the prior art, the preparation process of the isoquinolinone compounds of the present invention has the advantages of simple preparation process, short reaction time, high yield, few by-products, the results of scale-up production are good, and it has good industrialization prospect.

Intermediate

The present invention also provides an intermediate of the isoquinolinone compounds, and the structure of the intermediate of the isoquinolinone compounds is as shown in formula 3 or formula 4:

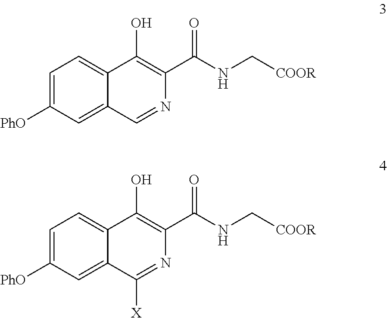

wherein, R is selected from C1-C10 alkyl, C6-C10 aryl, —R¹OR², wherein R¹ and R² are each independently selected from C1-C10 alkyl, and X is Cl, Br or I.

R is methyl, methoxymethyl, ethyl, ethoxyethyl, propyl, isopropyl, n-butyl, isobutyl, tert-butyl, benzyl, or phenyl.

In another preferred embodiment, X is Br.

In another preferred embodiment, the intermediate of the isoquinolinone compounds intermediate is:

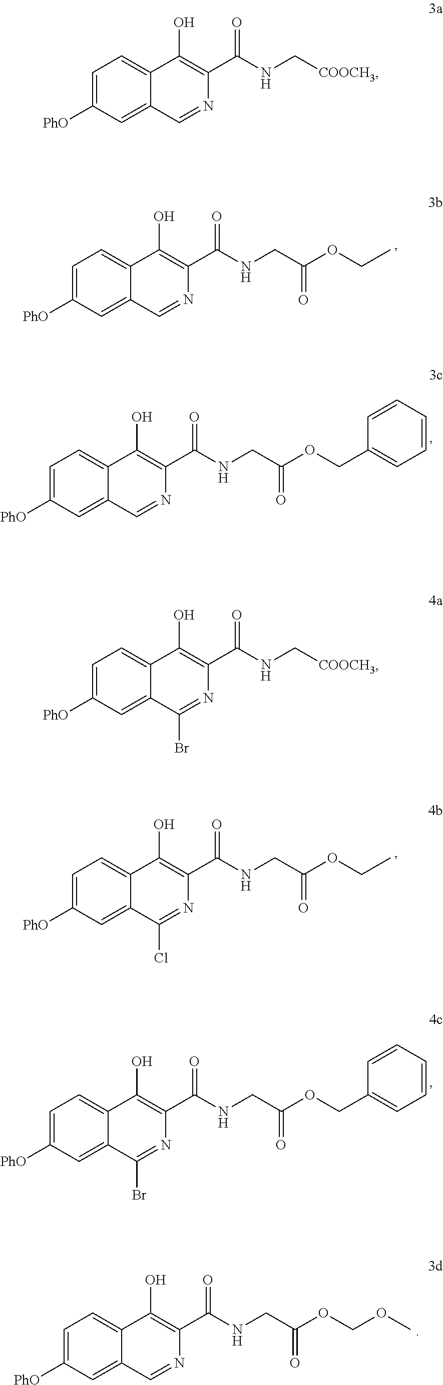

The Main Advantages of the Present Invention Include:

The preparation method of the isoquinolinone compounds of the present invention has the advantages of a reasonable route, convenient and easy operations, high preparation yield and high purity, and suitability for industrial production.

The present invention will be further illustrated below with reference to the specific examples. It should be understood that these examples are only to illustrate the invention but not to limit the scope of the invention. The experimental methods with no specific conditions described in the following examples are generally performed under the conventional conditions or according to the manufacturer's instructions. Unless indicated otherwise, percentages and parts are calculated by weight.

### EXAMPLE

In the examples, all reactions were carried out under normal pressure (standard normal atmosphere), and room temperature refers to 25±5° C.

### Example 1

### Example 1.1

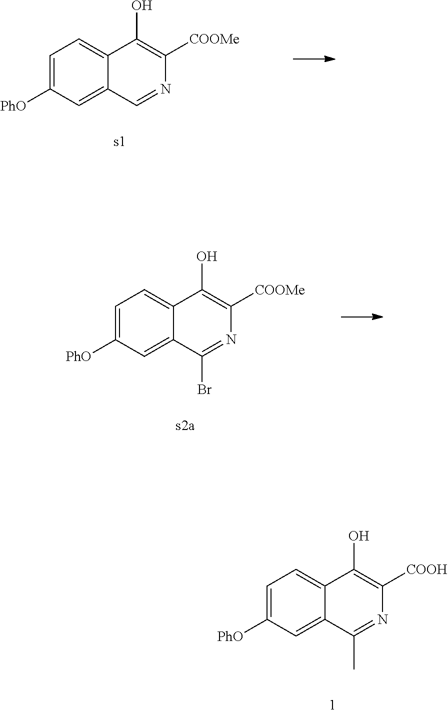 [SMILES: COC(=O)c1ncc2cc(Oc3ccccc3)ccc2c1O ; COC(=O)c1nc(Br)c2cc(Oc3ccccc3)ccc2c1O ; Cc1nc(C(=O)O)c(O)c2ccc(Oc3ccccc3)cc12]

Methyl 4-hydroxy-7-phenoxyisoquinolinyl-3-carboxylate (10 g, 33.87 mmol) was added to dichloromethane, and after cooling to 0-10° C., N-bromosuccinimide (NBS) solid (35.6 mmol) was added in batches. After the addition, the temperature was raised to room temperature and the reaction was carried out under stirring for 4-5 h. The completion of the reaction was detected by TLC plate. The mixture was concentrated to dryness, slurried with acetonitrile, and filtered to give 11.85 g of methyl 4-hydroxy-1-bromo-7-phenoxyisoquinolinyl-3-carboxylate with a yield of 93.9%.

The above methyl 4-hydroxy-1-bromo-7-phenoxyisoquinolinyl-3-carboxylate (7.0 g, 0.019 mol), tetrakis (triphenylphosphine) palladium (0.05 eq), methylboronic acid (1.5 eq), and potassium phosphate (2.0 eq) were added to 140 mL of ethylene glycol monomethyl ether and 28 mL of water in turn. The temperature was raised to 90-100° C. and the reaction was carried out for 3 h. The completion of the reaction was detected by TLC. The mixture was cooled to 20-30° C., then pure water was added. The pH of the mixture was adjusted to 2-3 by hydrochloric acid. After filtering, the obtained mixture was rinsed with methanol to give 5.1 g of 4-hydroxy-1-methyl-7-phenoxyisoquinolinyl-3-carboxylic acid with a yield of 91%.

### Example 1.2

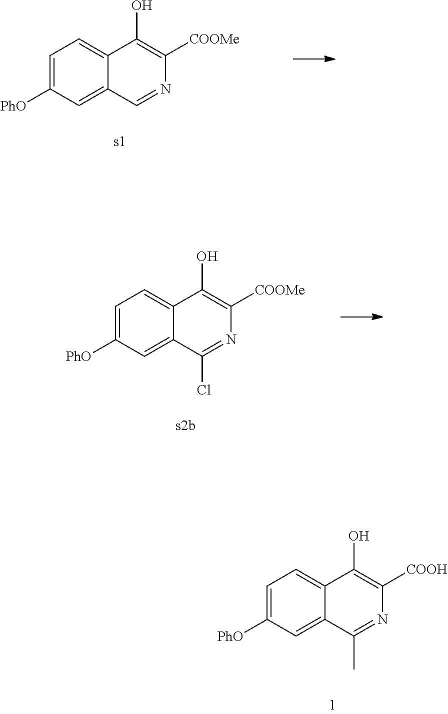 [SMILES: COC(=O)c1ncc2cc(Oc3ccccc3)ccc2c1O ; COC(=O)c1nc(Cl)c2cc(Oc3ccccc3)ccc2c1O ; Cc1nc(C(=O)O)c(O)c2ccc(Oc3ccccc3)cc12]

Methyl 4-hydroxy-7-phenoxyisoquinolinyl-3-carboxylate (10 g, 33.87 mmol) was added to acetonitrile. After the mixture was cooled to 0-10° C., N-chlorosuccinimide (NCS) solid (71.2 mmol) was added in batches. After the addition, the temperature was raised to room temperature and the reaction was carried out under stirring for 3.5-4.5 h. The completion of the reaction was detected by TLC plate. The mixture was concentrated to dryness, slurried with acetonitrile, and filtered to give 10.4 g of methyl 4-hydroxy-1-chloro-7-phenoxyisoquinolinyl-3-carboxylate with a yield of 92%.

The above methyl 4-hydroxy-1-chloro-7-phenoxyisoquinolinyl-3-carboxylate (6.2 g, 0.019 mol), triphenylphosphine palladium dichloride (0.05 eq), trimethylboron (1.5 eq) and Na₃PO4 (2.0 eq) were added to 124 mL of ethylene glycol monomethyl ether and 24.8 mL of water in turn. The temperature was raised to 90-100° C. and the reaction was carried out for 4 h. The completion of the reaction was detected by TLC. The mixture was cooled to 20-30° C., then pure water was added. The pH of the mixture was adjusted to 2-3 by hydrochloric acid. After filtering, the obtained mixture was rinsed with methanol to obtain 4.9 g of 4-hydroxy-1-methyl-7-phenoxyisoquinolinyl-3-carboxylic acid with a yield of 88%.

### Example 1.3

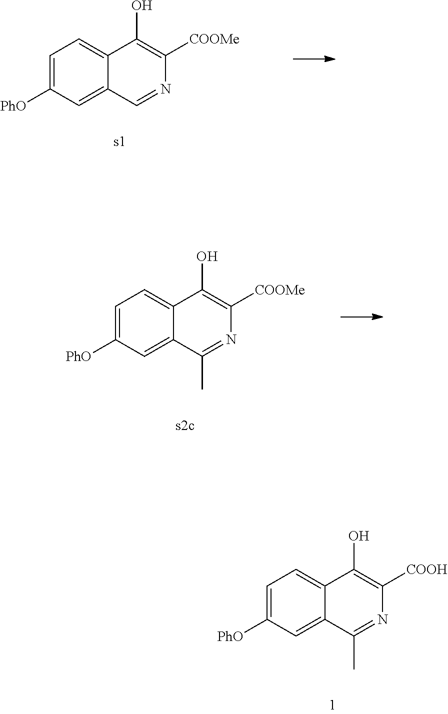 [SMILES: COC(=O)c1ncc2cc(Oc3ccccc3)ccc2c1O ; COC(=O)c1nc(C)c2cc(Oc3ccccc3)ccc2c1O ; Cc1nc(C(=O)O)c(O)c2ccc(Oc3ccccc3)cc12]

Methyl 4-hydroxy-7-phenoxyisoquinolinyl-3-carboxylate (10 g, 33.87 mmol) was added to 100 mL of acetonitrile and 100 mL of dichloromethane. After the mixture was cooled to 0-10° C., diiodohydantoin (90 mmol) was added in batches. After the addition, the temperature was raised to room temperature and the reaction was carried out under stirring for 4-4.5 h. The completion of the reaction was detected by TLC plate. The mixture was concentrated to dryness, slurried with acetonitrile, and filtered to give 11.45 g of methyl 4-hydroxy-1-iodo-7-phenoxyisoquinolinyl-3-carboxylate with a yield of 90.7%.

The above methyl 4-hydroxy-1-iodo-7-phenoxyisoquinolinyl-3-carboxylate (7.0 g, 0.019 mol), triphenylphosphine palladium dichloride (0.05 eq), isopropyl methylborate (1.5 eq) and K₂CO₃ (2.0 eq) were added to 105 mL of ethylene glycol monomethyl ether and 35 mL of water in turn. The temperature was raised to 90-100° C. and the reaction was carried out for 4 h. The completion of the reaction was detected by TLC. The mixture was cooled to 20-30° C., then pure water was added. The pH of the mixture was adjusted to 2-3 by hydrochloric acid. After filtering, the obtained solid was rinsed with methanol to give 5.3 g of 4-hydroxy-1-methyl-7-phenoxyisoquinolinyl-3-carboxylic acid with a yield of 95.4%.

### Example 1.4

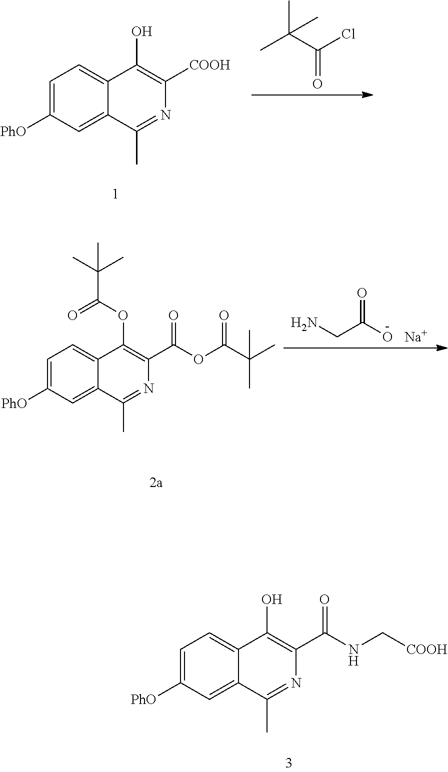

4-Hydroxy-1-methyl-7-phenoxyisoquinolinyl-3-carboxylic acid (2.0 g, 6.77 mmol) was added to 20 mL of tetrahydrofuran, followed by the addition of N, N-diisopropylethylamine (3.0 eq). The mixture was cooled to 0-10° C., and trimethylacetyl chloride (2.2 eq) was slowly added. After the addition, the temperature was raised to 20-30° C. and the reaction was carried out for 2-3 h. The completion of the reaction was detected by TLC to obtain the mixture containing pivalic acid-4-pivaloyloxy-1-methyl-7-phenoxyisoquinolinyl-3-carboxylic anhydride. The obtained mixture was directly subjected to the next step of the reaction, a small amount of the mixture was taken to be concentrated and filtered, and the filter residue was subjected to column chromatography to give the compound of formula 2a:

MS m/z (ESI) of the compound of formula 2: 464 (M+1); ¹H NMR (400 MHz, CDCl₃) δ 7.94 (dd, J=8.5, 1.1 Hz, 1H), 7.51 (s, 1H), 7.50-7.40 (m, 3H), 7.23 (d, J=7.4 Hz, 1H), 7.10 (dd, J=8.5, 0.8 Hz, 2H), 2.77 (s, 3H), 1.52 (s, 9H), 1.41 (s, 9H).

The mixture obtained above was cooled to 0-10° C. and sodium glycinate (2.0 eq, calculated based on the compound of formula 1) was slowly added. The temperature was raised to 20-30° C. and the reaction was carried out for 2-3 h. The completion of the reaction was detected by TLC. Then NaOH (2 eq, calculated based on the compound of formula 1) was added, and the mixture was stirred at room temperature for 2 h. Dichloromethane and water were used for extraction. The pH of the aqueous phase was adjusted to 2-3 with dilute hydrochloric acid and the solid was precipitated, filtered, rinsed with acetone, and dried to give 2.15 g of the compound product of formula 3 with a yield of 90%.

MS m/z (ESI) of the compound of formula 3: 353 (M+1); ¹H NMR (400 MHz, DMSO) δ 13.07 (d, J=196.2 Hz, 2H), 9.10 (t, J=5.9 Hz, 1H), 8.30 (d, J=9.0 Hz, 1H), 7.62 (d, J=2.3 Hz, 1H), 7.51 (ddd, J=15.9, 8.6, 5.0 Hz, 3H), 7.26 (t, J=7.4 Hz, 1H), 7.19 (d, J=7.7 Hz, 2H), 4.06 (d, J=6.1 Hz, 2H), 2.71 (s, 3H).

### Example 1.5

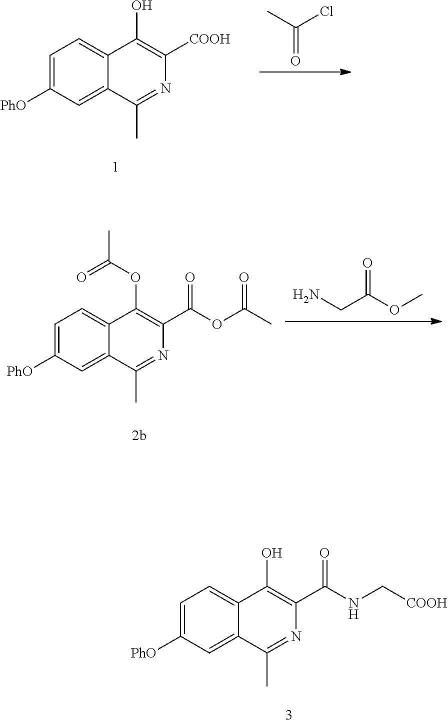

4-Hydroxy-1-methyl-7-phenoxyisoquinolinyl-3-carboxylic acid (2.0 g, 6.77 mmol) was added to 15 mL of dichloromethane followed by addition of triethylamine (4.0 eq). The mixture was cooled to 0-10° C. and acetyl chloride (3.2 eq) was slowly added. After the addition, the temperature was raised to 20-30° C. and the reaction was carried out for 1.5-2.5 h. The completion of the reaction was detected by TLC to obtain the mixture containing acetic acid-4-acetoxy-1-methyl-7-phenoxyisoquinolinyl-3-carboxylic anhydride. The obtained mixture was directly subjected to the next step of the reaction, a small amount of the mixture was taken to be concentrated and filtered, and the filter residue was subjected to column chromatography to give the compound of formula 2b:

MS m/z (ESI) of the compound of formula 2b: 380 (M+1). ¹H NMR (400 MHz, DMSO) δ 7.95 (dd, J=8.5, 1.1 Hz, 1H), 7.53 (s, 1H), 7.51-7.41 (m, 3H), 7.24 (d, J=7.4 Hz, 1H), 7.11 (dd, J=8.5, 0.8 Hz, 2H), 2.77 (s, 3H), 2.23 (s, 3H), 2.19 (s, 3H).

The mixture obtained above was cooled to 0-10° C., and methyl glycinate (2.0 eq, calculated based on the compound of formula 1) was slowly added. The temperature was raised to 30° C. and reaction was carried out for 3 h. The completion of the reaction was detected by TLC. Then KOH (2 eq, calculated based on the compound of formula 1) was added, and the mixture was stirred at room temperature for 2 h. Dichloromethane and water were used for extraction. The pH of the aqueous phase was adjusted to 2-3 with dilute hydrochloric acid and the solid was precipitated, filtered, rinsed with acetone, and dried to give 2.19 g of the compound product of formula 3 with a yield of 92%. The analytical data of the compound of formula 3 are the same as that in Example 1.4.

### Example 1.6

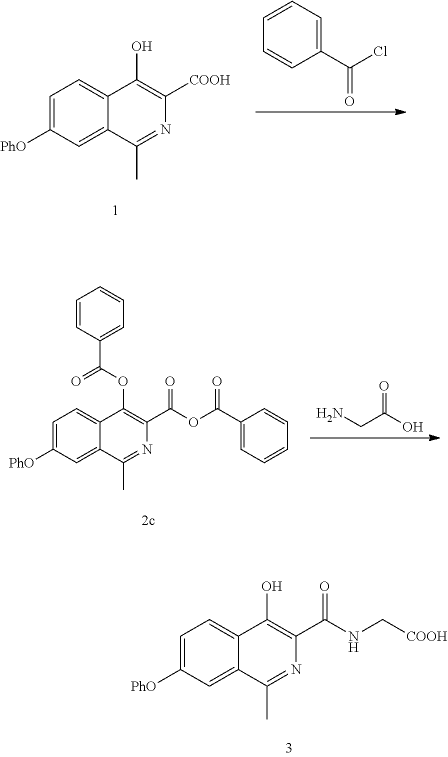 [SMILES: Cc1nc(C(=O)O)c(O)c2ccc(Oc3ccccc3)cc12 ; O=C(Cl)c1ccccc1 ; Cc1nc(C(=O)OC(=O)c2ccccc2)c(OC(=O)c2ccccc2)c2ccc(Oc3ccccc3)cc12 ; NCC(=O)O ; Cc1nc(C(=O)NCC(=O)O)c(O)c2ccc(Oc3ccccc3)cc12]

4-Hydroxy-1-methyl-7-phenoxyisoquinolinyl-3-carboxylic acid (2.0 g, 6.77 mmol) was added to 20 mL of toluene, followed by addition of DBU (2.0 eq). The mixture was cooled to 0-10° C. and benzoyl chloride (2.8 eq) was slowly added. The temperature was raised to 20-30° C. and the reaction was carried out for 2.5-3.5 h. The completion of the reaction was detected by TLC to obtain the mixture containing benzoic acid-4-benzoyloxy-1-methyl-7-phenoxyisoquinolinyl-3-carboxylic anhydride. The obtained mixture was directly subjected to the next step of the reaction, a small amount of the mixture was taken to be concentrated and filtered, and the filter residue was subjected to column chromatography to give the compound of formula 2c:

MS m/z (ESI) of the compound of formula 2c: 504 (M+1); ¹H NMR (400 MHz, DMSO) δ8.19 (dd, J=8.5, 0.8 Hz, 4H), 8.03 (dd, J=8.5, 1.1 Hz, 1H), 7.82-7.78 (m, 2H), 7.73-7.67 (m, 3H), 7.63-7.59 (m, 2H), 7.53-7.43 (m, 3H), 7.21 (d, J=7.4 Hz, 1H), 7.10 (dd, J=8.5, 0.8 Hz, 2H), 2.78 (s, 3H).

The mixture obtained above was cooled to 0-10° C., and glycine (2.0 eq, calculated based on the compound of formula 1) was slowly added. Then the temperature was raised to 30° C. and the reaction was carried out for 3 h. The completion of the reaction was detected by TLC. Then LiOH (3 eq, calculated based on the compound of formula 1) was added, and the mixture was stirred at room temperature to react for 2 h. Dichloromethane and water were used for extraction. The pH of the aqueous phase was adjusted to 2-3 with dilute hydrochloric acid and the solid was precipitated, filtered, rinsed with acetone, and dried to give 2.24 g of the compound product of formula 3 with a yield of 94%. The analytical data for the compound of formula 3 are the same as that in Example 1.4.

### Comparative Example 1

According to the synthetic method of Roxadustat disclosed by CN104024227, the specific route is as follows:

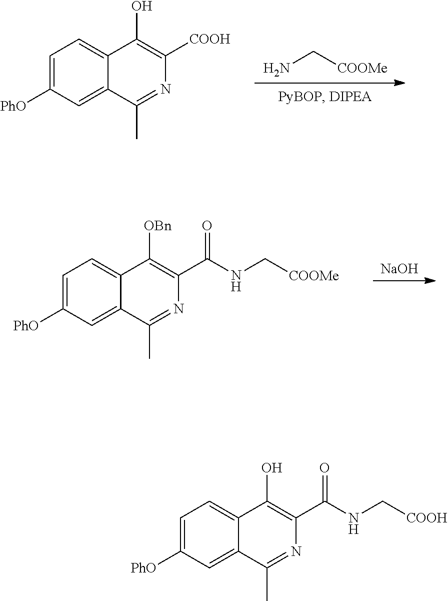 [SMILES: Cc1nc(C(=O)O)c(O)c2ccc(Oc3ccccc3)cc12 ; COC(=O)CN ; COC(=O)CNC(=O)c1nc(C)c2cc(Oc3ccccc3)ccc2c1OCc1ccccc1 ; "NaOH" ; Cc1nc(C(=O)NCC(=O)O)c(O)c2ccc(Oc3ccccc3)cc12]

The total yield of Roxadustat prepared by this route is 49.2%. The yield is low, and its application in practical production is limited.

### Example 2

### Example 2.1

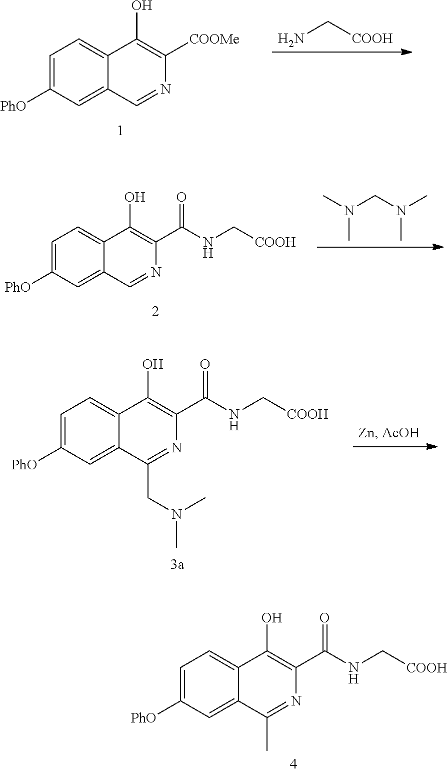

1) Methyl 4-hydroxy-7-phenoxyisoquinolinyl-3-carboxylate (20 g, 68 mmol) was added to acetonitrile, DBU (20.7 g, 136 mmol) was slowly added dropwise, then glycine (7.66 g, 102 mmol) was added. The temperature was raised to 50° C. and the reaction was carried out for 6 h. The completion of the reaction was detected by TLC plate. The mixture was cooled to room temperature and filtered. The filtrate was diluted with water and the pH is adjusted to be weakly acidic. The mixture was stirred to crystallization, filtered and baked to give 22 g of (4-hydroxy-7-phenoxyisoquinolinyl-3-carboxamido) acetic acid with a yield of 95.6% and a purity of 98.4% by HPLC. ¹H-NMR (400 MHz, CDCl₃): δ12.85 (s, 1H), 8.48-8.37 (m, 2H), 8.34 (d, J=9.0 Hz, 1H), 7.52-7.37 (m, 3H), 7.23 (d, J=7.4 Hz, 1H), 7.15-7.08 (m, 2H), 4.28 (d, J=5.8 Hz, 2H); MS m/z(ESI): 339 (M+1).

2) (4-Hydroxy-7-phenoxyisoquinolinyl-3-carboxamido) acetic acid (2.2 g, 6.5 mmol) and acetic acid were mixed, followed by slow addition of tetramethylmethanediamine (13 mmol). The atmosphere of the reaction system was replaced with argon, then the temperature was raised to 70° C. and the reaction was carried out for 3 h. The completion of the reaction was detected by TLC. The obtained reaction system was directly used in the next step without treatment. A small amount of reaction mixture was taken out, and water was added to the mixture to precipitate the solid to give the compound shown in formula 3a with the purity of 99% by HPLC. ¹H NMR (400 MHz, DMSO) δ 13.60 (s, 1H), 10.01 (s, 1H), 8.21 (d, J=9.1 Hz, 1H), 7.70 (d, J=2.0 Hz, 1H), 7.56-7.44 (m, 3H), 7.28 (t, J=7.4 Hz, 1H), 7.19 (d, J=7.7 Hz, 2H), 4.56 (s, 2H), 4.00 (d, J=6.1 Hz, 2H), 2.68 (s, 6H); MS m/z(ESI): 396 (M+1).

3) Zinc powder (4.2 g, 32.5 mmol) was added to the reaction system obtained in the previous step at room temperature to react at 70° C. for 5 h. The completion of the reaction was detected by TLC. The mixture was cooled to room temperature, filtered with suction. The filter cake was rinsed with acetic acid, and the filtrate was concentrated and treated with methyl tert-butyl ether to give 2.28 g of [(4-hydroxy-1-methyl-7-phenoxy-isoquinolinyl-3-carbonyl)-amino]-acetic acid with a yield of 99.1% and a purity of 99.5% by HPLC. ¹H NMR (400 MHz, DMSO) δ 13.07 (d, J=196.2 Hz, 2H), 9.10 (t, J=5.9 Hz, 1H), 8.30 (d, J=9.0 Hz, 1H), 7.62 (d, J=2.3 Hz, 1H), 7.51 (ddd, J=15.9, 8.6, 5.0 Hz, 3H), 7.26 (t, J=7.4 Hz, 1H), 7.19 (d, J=7.7 Hz, 2H), 4.06 (d, J=6.1 Hz, 2H), 2.71 (s, 3H); MS m/z(ESI): 353 (M+1).

### Example 2.2

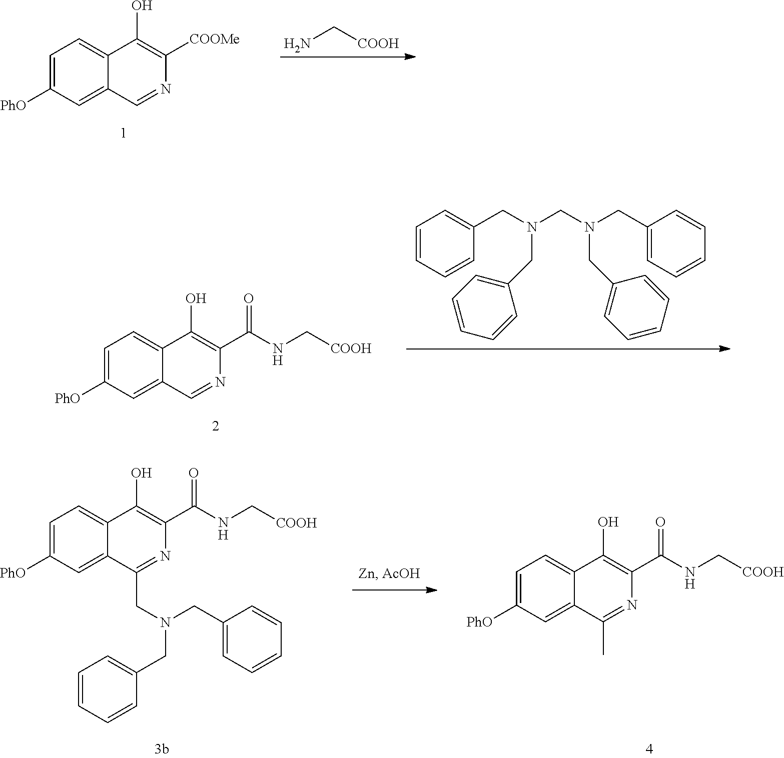 [SMILES: COC(=O)c1ncc2cc(Oc3ccccc3)ccc2c1O ; NCC(=O)O ; O=C(O)CNC(=O)c1ncc2cc(Oc3ccccc3)ccc2c1O ; c1ccc(CN(Cc2ccccc2)CN(Cc2ccccc2)Cc2ccccc2)cc1 ; O=C(O)CNC(=O)c1nc(CN(Cc2ccccc2)Cc2ccccc2)c2cc(Oc3ccccc3)ccc2c1O ; Cc1nc(C(=O)NCC(=O)O)c(O)c2ccc(Oc3ccccc3)cc12]

1) Methyl 4-hydroxy-7-phenoxyisoquinolinyl-3-carboxylate (20 g, 68 mmol) was added to DMSO and TEA (13.7 g, 136 mmol) was slowly added dropwise, followed by addition of glycine (10.2 g, 136 mmol). The temperature was raised to 65° C. and the reaction was carried out for 6 h. The completion of the reaction was detected by TLC plate. The mixture was cooled to room temperature and filtered. The filtrate was diluted with water and the pH was adjusted to be weakly acidic. The mixture was stirred to crystallization, filtered and baked to give 21.5 g of (4-hydroxy-7-phenoxyisoquinolinyl-3-carboxamido) acetic acid with a yield of 93.5% and a purity of 97.9% by HPLC.

2) (4-Hydroxy-7-phenoxyisoquinolinyl-3-carboxamido) acetic acid (2.2 g, 6.5 mmol) and trifluoroacetic acid were mixed, followed by slow addition of tetrabenzylmethyldiamine (26 mmol). The atmosphere of the reaction system was replaced with argon, then the temperature was raised to 100° C. and the reaction was carried out for 3 h. The completion of the reaction was detected by TLC. The obtained reaction system was directly used in the next step without treatment. A small amount of reaction mixture was taken out and added with water to precipitate the solid to give the compound shown in formula 3b with the purity of 99% by HPLC. MS m/z(ESI): 548(M+1). ¹H NMR (400 MHz, DMSO) δ 13.62 (s, 1H), 10.21 (s, 1H), 8.36 (d, J=9.1 Hz, 1H), 7.80 (d, J=2.0 Hz, 1H), 7.60-7.50 (m, 3H), 7.36-7.24 (m, 11H), 7.12 (d, J=7.7 Hz, 2H), 4.56 (s, 2H), 4.00 (s, 4H), 3.72 (d, J=6.1 Hz, 2H).

3) Zinc powder (8.4 g, 65 mmol) was added to the reaction system obtained in the previous step at room temperature and reacted at 120° C. for 5 h. The completion of the reaction was detected by TLC. The mixture was cooled to room temperature, filtered with suction. The filter cake was rinsed with acetic acid, and the filtrate was concentrated and treated with methyl tert-butyl ether to give 2.25 g of [(4-hydroxy-1-methyl-7-phenoxy-isoquinolinyl-3-carbonyl)-amino]-acetic acid with a yield of 98.7% and a purity of 98.8% by HPLC. The spectral data of the other substances are the same as that in Example 2.1.

### Example 2.3

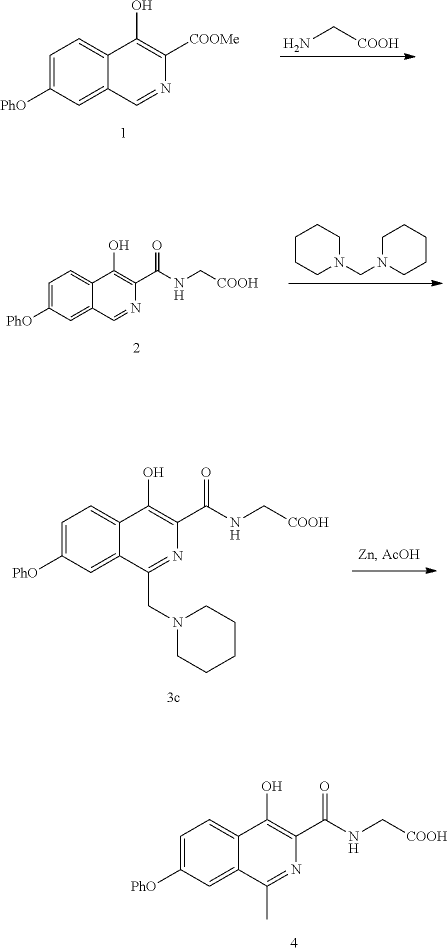 [SMILES: COC(=O)c1ncc2cc(Oc3ccccc3)ccc2c1O ; NCC(=O)O ; O=C(O)CNC(=O)c1ncc2cc(Oc3ccccc3)ccc2c1O ; C1CCN(CN2CCCCC2)CC1 ; O=C(O)CNC(=O)c1nc(CN2CCCCC2)c2cc(Oc3ccccc3)ccc2c1O ; Cc1nc(C(=O)NCC(=O)O)c(O)c2ccc(Oc3ccccc3)cc12]

1) Methyl 4-hydroxy-7-phenoxyisoquinolinyl-3-carboxylate (20 g, 68 mmol) was added to acetonitrile and pyridine (10.8 g, 136 mmol) was slowly added dropwise, followed by addition of glycine (7.66 g, 102 mmol). Then the temperature was raised to 100° C. and the reaction was carried out for 6 h. The completion of the reaction was detected by TLC plate. The mixture was cooled to room temperature and filtered. The filtrate was diluted with water and the pH was adjusted to be weakly acidic. The mixture was stirred to crystallization, filtered and baked to give 21 g of (4-hydroxy-7-phenoxyisoquinolinyl-3-carboxamido) acetic acid with a yield of 91.3% and a purity of 98% by HPLC.

2) (4-Hydroxy-7-phenoxyisoquinolinyl-3-carboxamido) acetic acid (2.2 g, 6.5 mmol) was added to 10 mL of acetic acid and 10 mL of isopropanol, followed by slow addition of dipiperidinylmethane (30 mmol). The atmosphere of the reaction system was replaced with argon, then the temperature was raised to 90° C. and the reaction was carried out for 3 h. The completion of the reaction was detected by TLC. The obtained reaction system was directly used in the next step without treatment. A small amount of reaction mixture was taken out and added with water to precipitate the solid to give the compound shown in formula 3c with a purity of 99% by HPLC. MS m/z(ESI): 436(M+1). ¹H NMR (400 MHz, DMSO) δ 13.60 (s, 1H), 10.03 (s, 1H), 8.23 (d, J=9.1 Hz, 1H), 7.70 (d, J=2.0 Hz, 1H), 7.56-7.44 (m, 3H), 7.28 (t, J=7.4 Hz, 1H), 7.19 (d, J=7.7 Hz, 2H), 4.26 (s, 2H), 3.68 (d, J=6.1 Hz, 2H), 2.84-2.44 (m, 4H), 1.66-1.50 (m, 4H), 1.46-1.38 (m, 2H).

3) Zinc powder (16.8 g, 130 mmol) was added to the reaction system obtained in the previous step at room temperature and reacted at 60° C. for 5 h. The completion of the reaction was detected by TLC. The mixture was cooled to room temperature, filtered with suction. The filter cake was rinsed with acetic acid, and the filtrate was concentrated and treated with methyl tert-butyl ether to give 2.1 g of [(4-hydroxy-1-methyl-7-phenoxy-isoquinolinyl-3-carbonyl)-amino]-acetic acid with a yield of 92.1% and a purity of 99.3% by HPLC. The spectral data of the other substances are the same as that in Example 2.1.

### Example 2.4

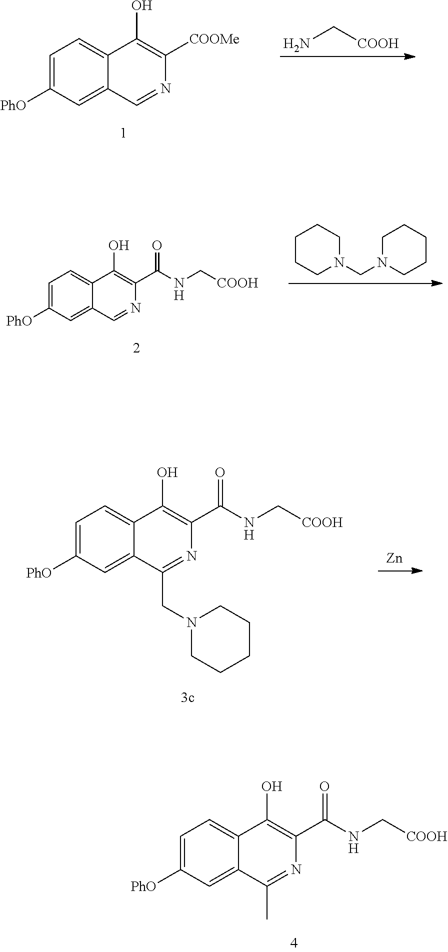

1) Methyl 4-hydroxy-7-phenoxyisoquinolinyl-3-carboxylate (20 g, 68 mmol) was added to acetonitrile, DBU (27.4 g, 272 mmol) was slowly added dropwise, followed by addition of glycine (7.66 g, 102 mmol). The temperature was raised to 100° C. and the reaction was carried out for 6 h. The completion of the reaction was detected by TLC plate. The mixture was cooled to room temperature and filtered. The filtrate was diluted with water and the pH was adjusted to be weakly acidic. The mixture was stirred to crystallization, filtered and baked to give 21 g of (4-hydroxy-7-phenoxyisoquinolinyl-3-carboxamido) acetic acid with a yield of 91.3% and a purity of 98.3% by HPLC.

2) (4-Hydroxy-7-phenoxyisoquinolinyl-3-carboxamido) acetic acid (2.2 g, 6.5 mmol) was added to the mixed solution of 10 mL of water and 1 mL of concentrated sulfuric acid, dipiperidinemethane (13 mmol) was slowly added with stirring. The atmosphere of the reaction system was replaced with argon, then the temperature was raised to 90° C. and the reaction was carried out for 5 h. The completion of the reaction was detected by TLC. The obtained reaction system was directly used in the next step without treatment. A small amount of reaction mixture was taken out and added with water to precipitate the solid to give the compound shown in formula 3c with the purity of 99% by HPLC.

3) Zinc powder (4.2 g, 32.5 mmol) and appropriate amount of sulfuric acid were added to the reaction system obtained in the previous step at room temperature and reacted at 90° C. for 5 h. The completion of the reaction was detected by TLC. The mixture was cooled to room temperature, filtered with suction. The filter cake was dried to give 2.25 g of [(4-hydroxy-1-methyl-7-phenoxy-isoquinolinyl-3-carbonyl)-amino]-acetic acid with a yield of 98.7% and a purity of 99.2% by HPLC. The spectral data of the substance is the same as that in Example 2.3.

### Example 2.5

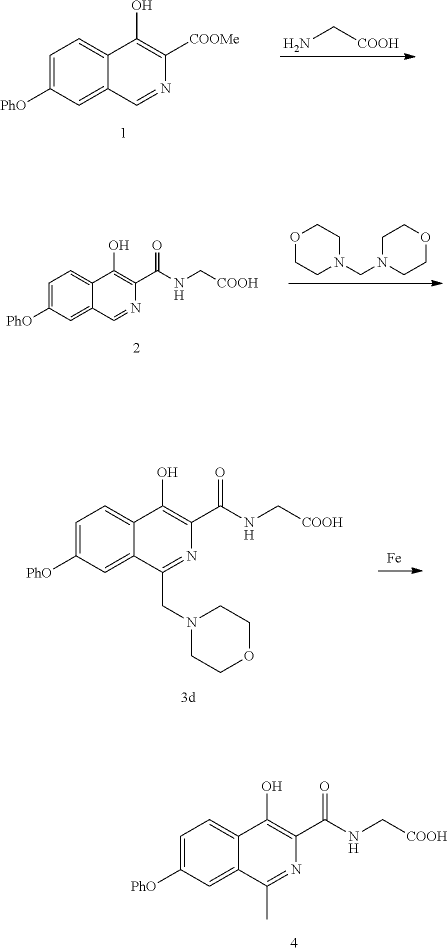

1) Methyl 4-hydroxy-7-phenoxyisoquinolinyl-3-carboxylate (20 g, 68 mmol) was added to acetonitrile, DBU (27.4 g, 272 mmol) was slowly added dropwise, followed by addition of glycine (7.66 g, 102 mmol). The temperature was raised to 100° C. and the reaction was carried out for 6 h. The completion of the reaction was detected by TLC plate. The mixture was cooled to room temperature and filtered. The filtrate was diluted with water and the pH was adjusted to be weakly acidic. The mixture was stirred to crystallization, filtered and baked to give 21 g of (4-hydroxy-7-phenoxyisoquinolinyl-3-carboxamido) acetic acid with a yield of 91.3% and a purity of 98.1% by HPLC.

2) (4-Hydroxy-7-phenoxyisoquinolinyl-3-carboxamido) acetic acid (2.2 g, 6.5 mmol) was added to a mixed solution of 15 mL of propionic acid and 5 mL of 1,4-dioxane, followed by slow addition of dimorpholinomethane (6.5 mmol). The atmosphere of the reaction system was replaced with argon, then the temperature was raised to 90° C. and the reaction was carried out for 3 h. The completion of the reaction was detected by TLC. The obtained reaction system was directly used in the next step without treatment. A small amount of reaction mixture was taken out and added with water to precipitate the solid to give the compound shown in formula 3d with the purity of 99% by HPLC. MS m/z(ESI): 438(M+1). ¹H NMR (400 MHz, DMSO) δ 13.61 (s, 1H), 10.02 (s, 1H), 8.22 (d, J=9.1 Hz, 1H), 7.70 (d, J=2.0 Hz, 1H), 7.56-7.44 (m, 3H), 7.28 (t, J=7.4 Hz, 1H), 7.19 (d, J=7.7 Hz, 2H), 4.28 (s, 2H), 3.70 (d, J=6.1 Hz, 2H), 3.44-3.34 (m, 4H), 2.87-2.42 (m, 4H).

3) Iron powder (4.2 g, 32.5 mmol) and appropriate amount of phosphoric acid were added to the reaction system obtained in the previous step at room temperature and reacted at 130° C. for 5 h. The completion of the reaction was detected by TLC. The mixture was cooled to room temperature, filtered with suction. The filter cake was rinsed with acetic acid, and the filtrate was concentrated and treated with methyl tert-butyl ether to give 2.15 g of [(4-hydroxy-1-methyl-7-phenoxy-isoquinolinyl-3-carbonyl)-amino]-acetic acid with a yield of 93% and a purity of 99.6% by HPLC. The spectral data of the other substances are the same as that in Example 2.1.

### Example 2.6

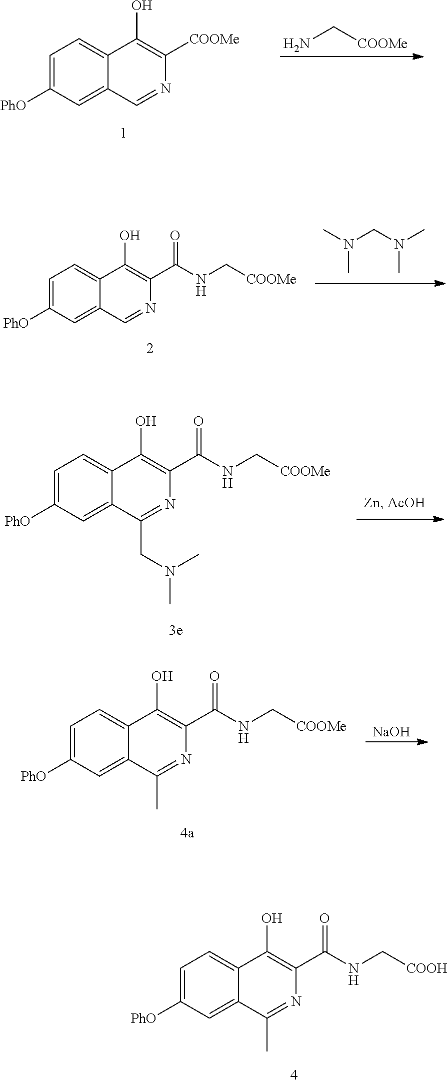

1) Methyl 4-hydroxy-7-phenoxyisoquinolinyl-3-carboxylate (20 g, 68 mmol) was added to acetonitrile and DBU (20.7 g, 136 mmol) was slowly added dropwise, followed by addition of methyl glycinate (9.09 g, 102 mmol). The temperature was raised to 50° C. and the reaction was carried out for 6 h. The completion of the reaction was detected by TLC plate. The mixture was cooled to room temperature and filtered. The filtrate was extracted with water and ethyl acetate, and the organic phase was concentrated to dryness under reduced pressure to give 21.6 g of methyl (4-hydroxy-7-phenoxyisoquinolinyl-3-carboxamido) acetate with a yield of 90% and a purity of 98.2% by HPLC. MS m/z(ESI): 353 (M+1). ¹H NMR (400 MHz, CDCl₃) δ 12.85 (s, 1H), 8.48-8.37 (m, 2H), 8.34 (d, J=9.0 Hz, 1H), 7.52-7.37 (m, 3H), 7.23 (d, J=7.4 Hz, 1H), 7.15-7.08 (m, 2H), 4.28 (d, J=5.8 Hz, 2H), 3.81 (s, 3H).

2) Methyl (4-hydroxy-7-phenoxyisoquinolinyl-3-carboxamido) acetate (2.2 g, 6.5 mmol) and acetic acid were mixed, followed by slow addition of tetramethylmethanediamine (13 mmol). The atmosphere of the reaction system was replaced with argon, then the temperature was raised to 80° C. and the reaction was carried out for 3 h. The completion of the reaction was detected by TLC. The obtained reaction system didn't need to be treated and directly subjected to the next step. A small amount of reaction mixture was taken out and added with water to precipitate the solid to give the compound shown in formula 3e with a purity of 99% by HPLC. MS m/z(ESI): 410 (M+1). ¹H NMR (400 MHz, DMSO) δ13.35 (s, 1H), 9.22 (s, 1H), 8.29 (d, J=9.1 Hz, 1H), 7.93 (d, J=2.1 Hz, 1H), 7.53 (m, J=19.4, 13.4, 8.1 Hz, 3H), 7.28 (t, J=7.4 Hz, 1H), 7.21 (d, J=7.7 Hz, 2H), 4.15 (d, J=6.1 Hz, 2H), 3.79 (s, 2H), 3.69 (s, 3H), 2.12 (s, 6H).

3) Zinc powder (4.2 g, 32.5 mmol) was added to the reaction system obtained in the previous step at room temperature and reacted at 80° C. for 5 h. The completion of the reaction was detected by TLC. The mixture was cooled to room temperature, filtered with suction. The filter cake was rinsed with acetic acid, and the filtrate was concentrated and treated with methyl tert-butyl ether to give 2.28 g of methyl [(4-hydroxy-1-methyl-7-phenoxy-isoquinolinylyl-3-carbonyl)-amino]-acetate with a yield of 99.1% and a purity of 98.5% by HPLC. MS m/z(ESI): 367 (M+1). ¹H NMR (400 MHz, DMSO) δ 13.22 (s, 1H), 9.24 (t, J=6.2 Hz, 1H), 8.30 (d, J=9.0 Hz, 1H), 7.63 (d, J=2.3 Hz, 1H), 7.51 (i, J=9.9, 4.2, 2.3 Hz, 3H), 7.27 (dd, J=9.3, 5.5 Hz, 1H), 7.18 (i, J=10.1, 5.2 Hz, 2H), 4.15 (d, J=6.2 Hz, 2H), 3.69 (s, 3H), 2.72 (s, 3H).

4) Methyl [(4-hydroxy-1-methyl-7-phenoxy-isoquinolinyl-3-carbonyl)-amino]-acetate(2 g, 5.46 mmol) and sodium hydroxide (0.328 g, 8.19 mmol) were added to 20 mL of methanol and reacted under stirring at room temperature for 4 h. The completion of the reaction was detected by TLC. The pH of the mixture was adjusted to be weakly acidic with 2N hydrochloric acid to precipitate solid. After filtering, 1.88 g of [(4-hydroxy-1-methyl-7-phenoxy-isoquinolinyl-3-carbonyl)-amino]-acetic acid was obtained with a yield of 98% and a purity of 98.6% by HPLC. The spectral data are the same as that in Example 2.1.

### Example 2.7

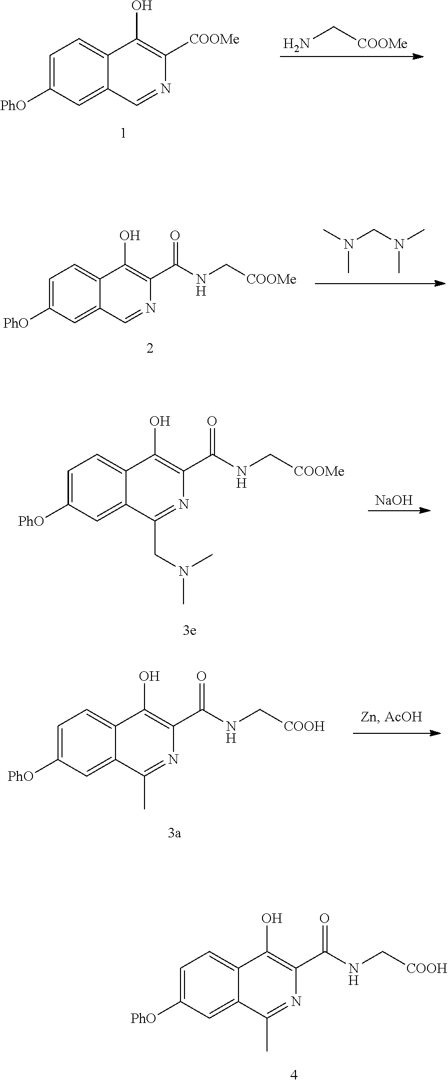

1) Methyl 4-hydroxy-7-phenoxyisoquinolinyl-3-carboxylate (20 g, 68 mmol) was added to acetonitrile and DBU (20.7 g, 136 mmol) was slowly added dropwise, followed by addition of methyl glycinate (9.09 g, 102 mmol). The temperature was raised to 50° C. and the reaction was carried out for 6 h. The completion of the reaction was detected by TLC plate. The mixture was cooled to room temperature and filtered. The filtrate was extracted with water and ethyl acetate, and the organic phase was concentrated to dryness under reduced pressure to give 21.6 g of methyl (4-hydroxy-7-phenoxyisoquinolinyl-3-carboxamido) acetate with a yield of 90% and a purity of 98.7% by HPLC.

2) Methyl (4-hydroxy-7-phenoxyisoquinolinyl-3-carboxamido) acetate (2.2 g, 6.5 mmol) and acetic acid were mixed, followed by slow addition of tetramethylmethanediamine (13 mmol). The atmosphere of the reaction system was replaced with argon, then the temperature was raised to 80° C. and the reaction was carried out for 3 h. The completion of the reaction was detected by TLC. The reaction solution was extracted with water and ethyl acetate, and the organic phase was concentrated to dryness to give 2.53 g of methyl (1-((dimethylamino)methyl) 4-hydroxy-7-phenoxyisoquinolinyl-3-carboxamido) acetate (3e) with a yield of 95% and a purity of 98.3% by HPLC.

3) Methyl (1-((dimethylamino) methyl) 4-hydroxy-7-phenoxyisoquinolinyl-3-carboxamido) acetate (2.5 g, 6.1 mmol) and sodium hydroxide (0.366 g, 9.15 mmol) were added to 25 mL of methanol and reacted under stirring at room temperature for 4 h. The completion of the reaction was detected by TLC. The pH of the mixture was adjusted to be weakly acidic with 2N hydrochloric acid to precipitate solid. After filtering, 2.36 g of (1-((dimethylamino)methyl)4-hydroxy-7-phenoxyisoquinolinyl-3-carboxamido) acetic acid was obtained with a yield of 98% and a purity of 98.1% by HPLC.

4) (1-((Dimethylamino) methyl) 4-hydroxy-7-phenoxyisoquinolinyl-3-carboxamido) acetic acid (2 g, 5.06 mmol) was added to 20 mL of acetic acid, followed by addition of zinc powder (3.3 g, 50.6 mmol) and reacted at 80° C. for 5 h. The completion of the reaction was detected by TLC. The mixture was cooled to room temperature, filtered with suction. The filter cake was rinsed with acetic acid, and the filtrate was concentrated and treated with methyl tert-butyl ether to give 1.73 g of methyl [(4-hydroxy-1-methyl-7-phenoxy-isoquinolinyl-3-carbonyl)-amino]-acetate with a yield of 97% and a purity of 98.3% by HPLC. The spectral data of each substance is correspondingly the same as that in Example 2.1 or Example 2.6.

### Comparative Example 2

According to the synthetic method of Roxadustat disclosed by CN103435546, the specific route is as follows (d-g steps in Example 2.3):

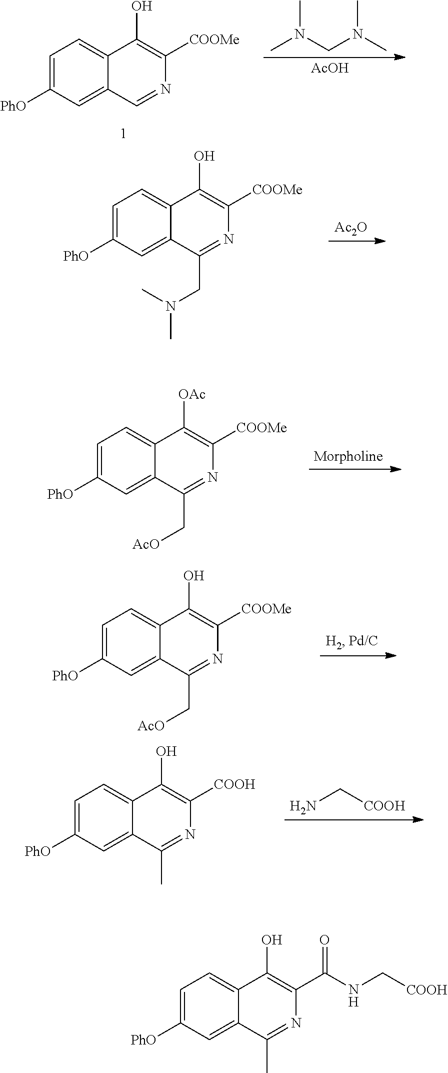

The total yield of this route is 62.1%. Moreover, the palladium carbon catalysis and ammonolysis steps need to be pressurized, which have high requirements for equipment in industrial production, require sealing pipes or other pressure-resistant equipments and have a certain degree of safety hazard.

### Example 3

### Example 3.1

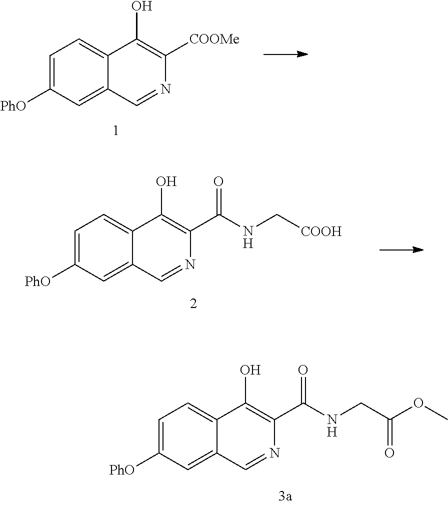 [SMILES: COC(=O)c1ncc2cc(Oc3ccccc3)ccc2c1O ; O=C(O)CNC(=O)c1ncc2cc(Oc3ccccc3)ccc2c1O ; COC(=O)CNC(=O)c1ncc2cc(Oc3ccccc3)ccc2c1O]

1) Methyl 4-hydroxy-7-phenoxyisoquinolinyl-3-carboxylate (20 g, 68 mmol) was added to acetonitrile and DBU (20.7 g, 136 mmol) was slowly added dropwise, followed by addition of glycine (7.66 g, 102 mmol). The temperature was raised to 65° C. and the reaction was carried out for 6 h. The completion of the reaction was detected by TLC plate. The mixture was cooled to room temperature and filtered. The pH of the filtrate is adjusted to be weakly acidic. The mixture was stirred to crystallization, filtered and baked to give 22 g of (4-hydroxy-7-phenoxyisoquinolinyl-3-carboxamido) acetic acid with a yield of 96% and a purity of 98.10% by HPLC.

(4-Hydroxy-7-phenoxyisoquinolinyl-3-carboxamido) acetic acid (8 g, 24 mmol) was mixed with 120 mL of methanol and the mixture was cooled to 10° C., followed by slow addition of oxalyl chloride (91.5 mmol). After the addition, the temperature was raised to reflux, and the reaction was carried out for 3 h. The completion of the reaction was detected by TLC. The mixture was concentrated to dryness and extracted with dichloromethane, washed with water, dried and filtered, and then concentrated to a minimum amount. The crystallization was performed by dropwise addition of petroleum ether. The mixture was filtered and dried to give 8.2 g of methyl (4-hydroxy-7-phenoxyisoquinolinyl-3-carboxamido) acetate with a yield of 98% and a purity of 97.7% by HPLC. ¹H NMR (400 MHz, CDCl₃): δ 12.85 (s, 1H), 8.48-8.37 (m, 2H), 8.34 (d, J=9.0 Hz, 1H), 7.52-7.37 (m, 3H), 7.23 (d, J=7.4 Hz, 1H), 7.15-7.08 (m, 2H), 4.28 (d, J=5.8 Hz, 2H), 3.81 (s, 3H).

### Example 3.2

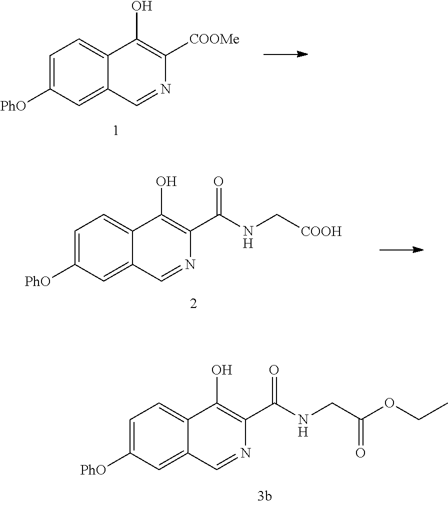 [SMILES: COC(=O)c1ncc2cc(Oc3ccccc3)ccc2c1O ; O=C(O)CNC(=O)c1ncc2cc(Oc3ccccc3)ccc2c1O ; CCOC(=O)CNC(=O)c1ncc2cc(Oc3ccccc3)ccc2c1O]

Methyl 4-hydroxy-7-phenoxyisoquinolinyl-3-carboxylate (20 g, 68 mmol) was added to NMP, DIEA (252 mmol) was slowly added dropwise, followed by addition of glycine (15.3 g, 204 mmol). After the addition, the temperature was raised to 100° C. and the reaction was carried out for 6 h. The completion of the reaction was detected by TLC plate. The mixture was cooled to room temperature and filtered. The pH of the filtrate was adjusted to be weakly acidic. The mixture was stirred to crystallization, filtered and baked to give 21 g of (4-hydroxy-7-phenoxyisoquinolinyl-3-carboxamido) acetic acid with a yield of 91% and a purity of 98.3% by HPLC.

(4-Hydroxy-7-phenoxyisoquinolinyl-3-carboxamido) acetic acid (8 g, 24 mmol) was mixed with 120 mL of ethanol and the mixture was cooled to 0° C., followed by slow addition of thionyl chloride (72 mmol). After the addition, the temperature was raised to reflux, and the reaction was carried out for 3 h. The completion of the reaction was detected by TLC. The mixture was concentrated to dryness and extracted with dichloromethane, washed with water, dried and filtered, then concentrated to a minimum amount. The crystallization was performed by dropwise addition of petroleum ether. The mixture was filtered and dried to give 8.4 g of ethyl (4-hydroxy-7-phenoxyisoquinolinyl-3-carboxamido) acetate with a yield of 97% and a purity of 96.4% by HPLC.

### Example 3.3

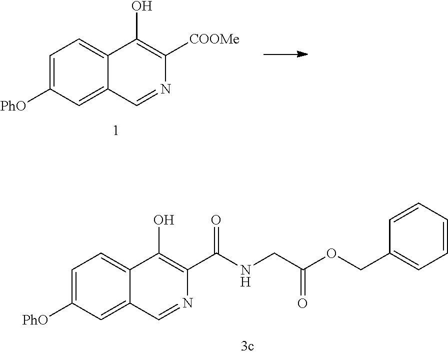 [SMILES: COC(=O)c1ncc2cc(Oc3ccccc3)ccc2c1O ; O=C(CNC(=O)c1ncc2cc(Oc3ccccc3)ccc2c1O)OCc1ccccc1]

Methyl 4-hydroxy-7-phenoxyisoquinolinyl-3-carboxylate (10 g, 33.9 mmol) was mixed with tetrahydrofuran, DIEA (136 mmol) was slowly added dropwise, followed by addition of benzyl glycinate (136 mmol). After the addition, the temperature was raised to 65° C. and the reaction was carried out for 6 h. The completion of the reaction was detected by TLC plate. The mixture was cooled to room temperature and then extracted with water and ethyl acetate. The organic phase was concentrated to give 15 g of benzyl (4-hydroxy-7-phenoxyisoquinolinyl-3-carboxamido) acetate with a yield of 83.8% and a purity of 97.3% by HPLC.

### Example 3.4

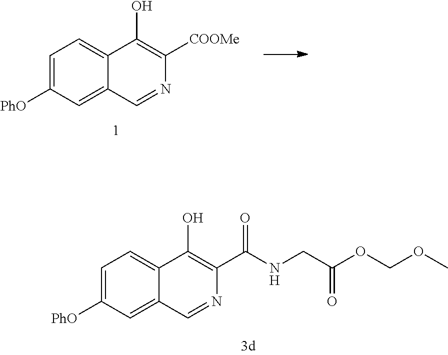 [SMILES: COC(=O)c1ncc2cc(Oc3ccccc3)ccc2c1O ; COCOC(=O)CNC(=O)c1ncc2cc(Oc3ccccc3)ccc2c1O]

Methyl 4-hydroxy-7-phenoxyisoquinolinyl-3-carboxylate (10 g, 33.9 mmol) was mixed with acetonitrile, DBU (10.3 g, 67.7 mmol) was slowly added dropwise, followed by addition of methoxymethyl glycinate (68 mmol). After the addition, the temperature was raised to 65° C. and the reaction was carried out for 6 h. The completion of the reaction was detected by TLC plate. The mixture was cooled to room temperature and then extracted with water and ethyl acetate. The organic phase was concentrated to give 10 g of methoxymethyl (4-hydroxy-7-phenoxyisoquinolinyl-3-carboxamido) acetate with a yield of 83.8% and a purity of 98.0% by HPLC.

### Example 3.5

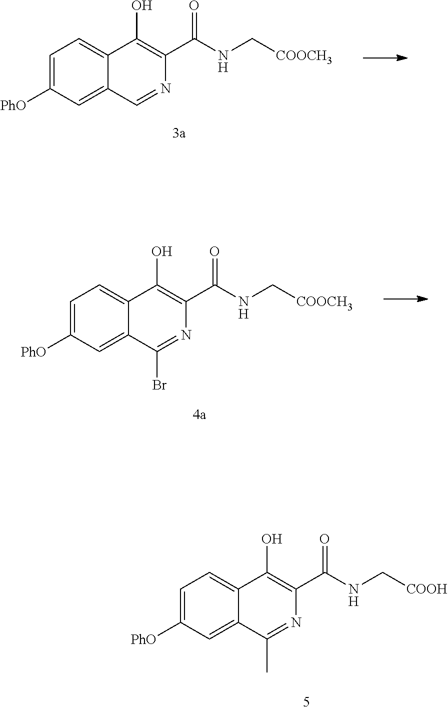 [SMILES: COC(=O)CNC(=O)c1ncc2cc(Oc3ccccc3)ccc2c1O ; COC(=O)CNC(=O)c1nc(Br)c2cc(Oc3ccccc3)ccc2c1O ; Cc1nc(C(=O)NCC(=O)O)c(O)c2ccc(Oc3ccccc3)cc12]

Methyl (4-hydroxy-7-phenoxyisoquinolinyl-3-carboxamido) acetate (26 g, 73.8 mmol) was mixed with dichloromethane and cooled to 0-10° C., followed by dropwise addition of NBS (88.5 mmol). After the addition, the temperature was raised to room temperature and the reaction was carried out for 3 h. The completion of the reaction was detected by TLC. The reaction mixture was washed with sodium bisulfite solution, dried and concentrated to give 26 g of methyl (4-hydroxy-1-bromo-7-phenoxyisoquinolinyl-3-carboxamido) acetate with a yield of 81% and a purity of 96.5% by HPLC. ¹H NMR (400 MHz, CDCl₃) δ 12.83 (s, 1H), 8.34 (d, J=9.0 Hz, 1H), 8.15 (t, J=5.5 Hz, 1H), 7.64 (d, J=2.3 Hz, 1H), 7.52-7.40 (m, 3H), 7.24 (d, J=7.5 Hz, 1H), 7.17-7.09 (m, 2H), 4.27 (d, J=5.9 Hz, 2H), 3.82 (s, 3H).

Methyl (4-hydroxy-1-bromo-7-phenoxyisoquinolinyl-3-carboxamido) acetate (20 g, 46.4 mmol), methylboronic acid (7.5 g, 125.2 mmol), potassium phosphate (27.6 g, 130 mmol) and bis(triphenylphosphorus) palladium dichloride (2.6 g, 3.7 mmol) were mixed with 400 mL of ethylene glycol monomethyl ether, followed by the addition of 80 mL of water. The temperature was raised to 110° C. and the reaction was carried out for 4 h. The completion of the reaction was detected by TLC. The mixture was cooled to room temperature and filtered, added with 400 mL of water. The pH of the solution was adjusted to 3-4 for crystallization. The mixture was filtered and the filter cake was dried to give 15.0 g of [(4-hydroxy-1-methyl-7-phenoxy-isoquinolinyl-3-carbonyl)-amino]-acetic acid with a yield of 91% and a purity of 97.7% by HPLC. ¹H NMR (400 MHz, DMSO): δ 13.07 (d, J=196.2 Hz, 2H), 9.10 (t, J=5.9 Hz, 1H), 8.30 (d, J=9.0 Hz, 1H), 7.62 (d, J=2.3 Hz, 1H), 7.51 (ddd, J=15.9, 8.6, 5.0 Hz, 3H), 7.26 (t, J=7.4 Hz, 1H), 7.19 (d, J=7.7 Hz, 2H), 4.06 (d, J=6.1 Hz, 2H), 2.71 (s, 3H).

### Example 3.6

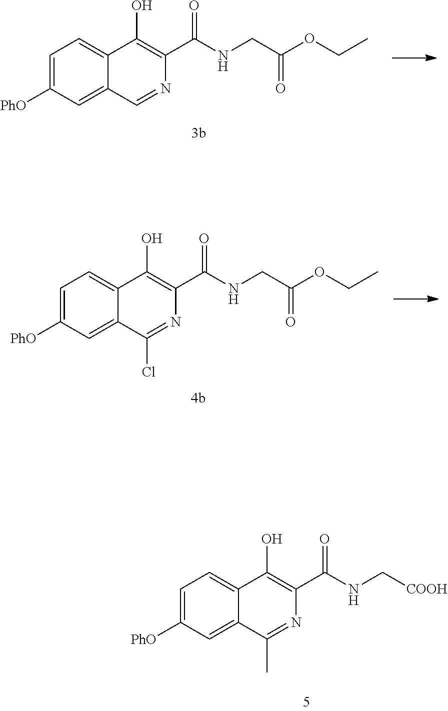 [SMILES: CCOC(=O)CNC(=O)c1ncc2cc(Oc3ccccc3)ccc2c1O ; CCOC(=O)CNC(=O)c1nc(Cl)c2cc(Oc3ccccc3)ccc2c1O ; Cc1nc(C(=O)NCC(=O)O)c(O)c2ccc(Oc3ccccc3)cc12]

Ethyl (4-hydroxy-7-phenoxyisoquinolinyl-3-carboxamido) acetate (27 g, 73.8 mmol) was mixed with dichloromethane. The temperature was cooled to 0-10° C., followed by dropwise addition of NCS (160 mmol). After the addition, the temperature was raised to room temperature and the reaction was carried out for 3 h. The completion of the reaction was detected by TLC. The reaction mixture was washed with sodium bisulfite solution, dried and concentrated to give 26 g of methyl (4-hydroxy-1-chloro-7-phenoxyisoquinolinyl-3-carboxamido) acetate with a yield of 87% and a purity of 96.8% by HPLC.

Ethyl (4-hydroxy-1-chloro-7-phenoxyisoquinolinyl-3-carboxamido) acetate (17 g, 46.4 mmol), trimethylboron (192 mmol), potassium carbonate (130 mmol), and tetrakis(triphenylphosphine)palladium (3.7 mmol) were mixed with 300 mL of ethanol, followed by the addition of 80 mL of water. The temperature was raised to 110° C. and the reaction was carried out for 4 h. The completion of the reaction was detected by TLC. The mixture was cooled to room temperature and filtered, added with 400 mL of water. The pH of the solution was adjusted to 3-4 for crystallization. The mixture was filtered and the filter cake was dried to give 14.2 g of [(4-hydroxy-1-methyl-7-phenoxy-isoquinolinyl-3-carbonyl)-amino]-acetic acid with a yield of 86% and a purity of 96.3% by HPLC.

### Example 3.7

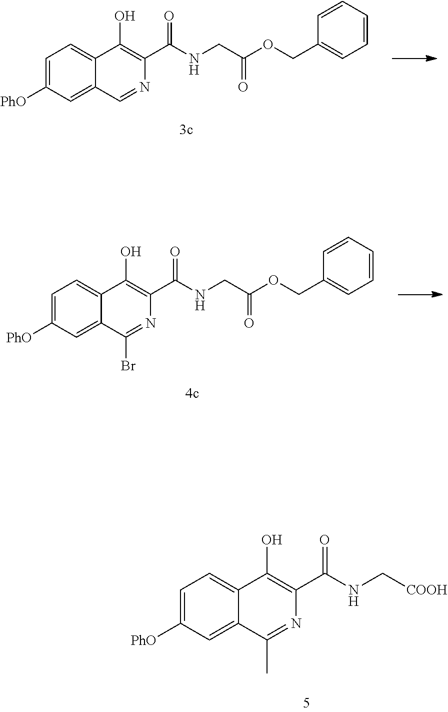 [SMILES: O=C(CNC(=O)c1ncc2cc(Oc3ccccc3)ccc2c1O)OCc1ccccc1 ; O=C(CNC(=O)c1nc(Br)c2cc(Oc3ccccc3)ccc2c1O)OCc1ccccc1 ; Cc1nc(C(=O)NCC(=O)O)c(O)c2ccc(Oc3ccccc3)cc12]

Benzyl (4-hydroxy-7-phenoxyisoquinolinyl-3-carboxamido) acetate (31.5 g, 73.8 mmol) was mixed with 315 mL of dichloromethane and 315 mL of acetonitrile. The temperature was cooled to 0-10° C., followed by dropwise addition of dibromohydantoin (300 mmol). After the addition, the temperature was raised to room temperature and the reaction was carried out for 3 h. The completion of the reaction was detected by TLC. The reaction mixture was washed with sodium bisulfite solution, dried and concentrated to give 25 g of benzyl (4-hydroxy-1-bromo-7-phenoxyisoquinolinyl-3-carboxamido) acetate with a yield of 81% and a purity of 96.8% by HPLC.

Benzyl (4-hydroxy-1-bromo-7-phenoxyisoquinolinyl-3-carboxamido) acetate (23.4 g, 46.4 mmol), isopropyl methylborate (150 mmol), potassium acetate (185.6 mmol), and triphenylphosphine palladium acetate (3.7 mmol) were mixed with 200 mL of ethylene glycol monomethyl ether and 300 mL of ethanol, followed by the addition of 80 mL of water. The temperature was raised to 110° C. and the reaction was carried out for 4 h. The completion of the reaction was detected by TLC. The mixture was cooled to room temperature and filtered. After adding 400 mL of water, the pH of the solution was adjusted to 3-4 for crystallization. The mixture was filtered and the filter cake was dried to give 15 g of [(4-hydroxy-1-methyl-7-phenoxy-isoquinolinyl-3-carbonyl)-amino]-acetic acid with a yield of 92% and a purity of 96.9% by HPLC.

### Comparative Example 3

According to the synthetic method of Roxadustat disclosed by CN107954931, the specific route is as follows:

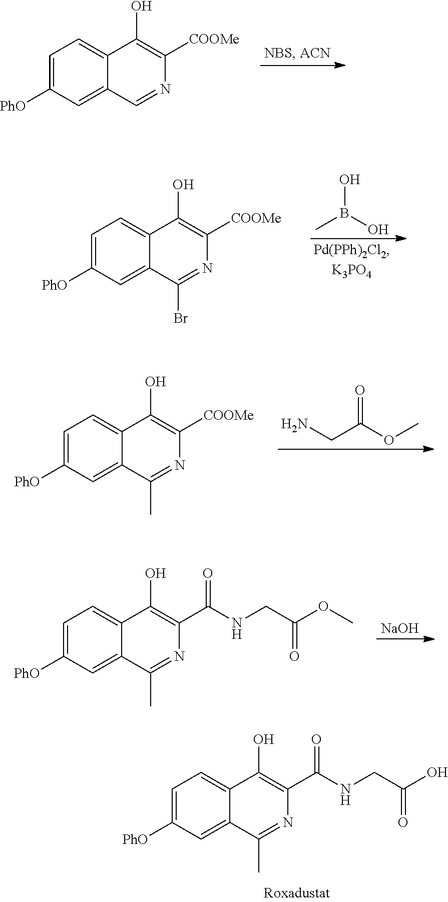

The yield of this route was 49.3%.

Compared with the comparative examples, since the raw material is first subjected to aminolysis in the method of the examples of the present invention, not only can the reaction rate be speed up in the subsequent suzuki coupling process, but also the use of high-pressure reaction conditions or complicated coupling agents in the process of aminolysis after methylation is avoided, which increases industrial productivity and reduces costs.

All documents mentioned herein are incorporated by reference in this application as if each document were individually incorporated by reference. In addition, it should be understood that after reading the above teaching content of the present invention, those skilled in the art can make various changes or modifications to the present invention, and these equivalent forms also fall within the scope defined by the appended claims of the present application.

1. A method for preparing a compound of formula 3, wherein the method comprises the following steps:
1) reacting a compound of formula 1 with acyl chloride to obtain a compound of formula 2; 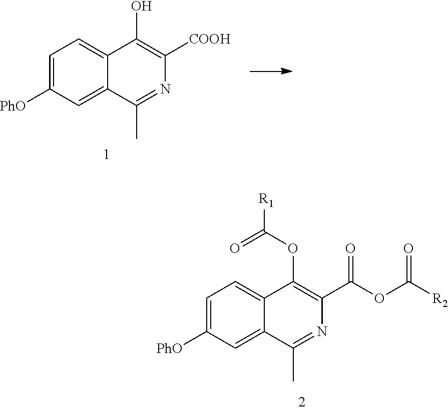
2) reacting the compound of formula 2 with an aminolysis reagent, followed by a hydrolysis reaction, to obtain the compound of formula 3, wherein the aminolysis reagent is selected from the group consisting of glycine, glycine derivatives, and a combination thereof, 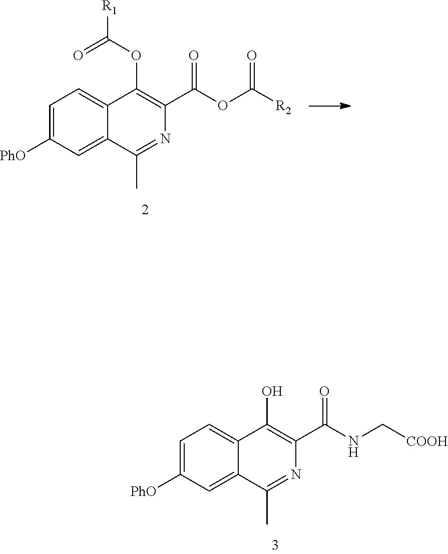
wherein, the acyl chloride is selected from the group consisting of R₁C(O)Cl, R₂C(O)Cl, and a combination thereof,
R₁ and R₂ are each independently C1-C10 alkyl or C6-C10 aryl.

2. The method according to claim 1, wherein,
in the step 1), the compound of formula 1 is reacted with the acyl chloride under the action of a deacid reagent and in a first inert solvent to obtain the compound of formula 2; and/or
in the step 2), the compound of formula 2 is first aminated by the aminolysis reagent, and then hydrolyzed by a first basic reagent to obtain the compound of formula 3.

3. The method according to claim 2, wherein the method comprises one or more features selected from the group consisting of:
in the step 1), the deacid reagent is selected from the group consisting of triethylamine (TEA), 1,8-diazabicycloundec-7-ene (DBU), N,N-diisopropylethylamine (DIEA), pyridine, N-methylmorpholine, and a combination thereof,
the first inert solvent in the step 1) is selected from the group consisting of tetrahydrofuran, dichloromethane, toluene, and a combination thereof,
in the step 1), the acyl chloride is selected from the group consisting of acetyl chloride, trimethylacetyl chloride, benzoyl chloride, and a combination thereof;
in the step 1), the molar ratio of the acyl chloride to the compound of formula 1 is 1-4:1;
in the step 2), the first basic reagent is selected from the group consisting of sodium hydroxide, potassium hydroxide, lithium hydroxide, and a combination thereof,
in the step 2), the glycine derivative comprises glycinate (salt) or glycinate (ester);
R₁ and R₂ are each independently methyl, ethyl, n-propyl, phenyl, benzyl, n-butyl, isobutyl, or tert-butyl;
in the step 2), the molar ratio of the aminolysis reagent to the compound of formula 1 is 1-3:1;
in the step 1), the molar ratio of the compound of formula 1 to the deacid reagent is 1:1-6; and/or
in the step 2), the molar ratio of the compound of formula 2 to the first basic reagent is 1:1-6.

4. The method according to claim 2, wherein the glycine derivative is selected from the group consisting of sodium glycinate, methyl glycinate, and a combination thereof; and/or
the first basic reagent comprises sodium hydroxide.

5. The method according to claim 1, wherein the compound of formula 1 is prepared by the following method:
(a) reacting a compound of formula s1 with a halogenated reagent in a second inert solvent to obtain a compound of formula s2;
(b) reacting the compound of formula s2 with a methylating reagent in a third inert solvent in the presence of a palladium catalyst and a second basic reagent to obtain the compound of formula 1; 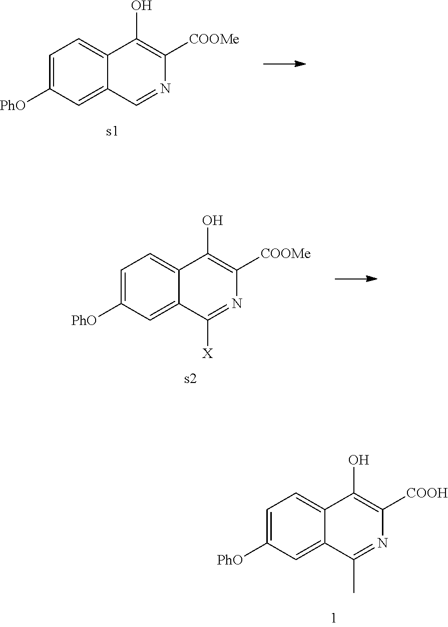
wherein, x is Cl, Br or I.

6. A method for preparing a compound of formula 4, wherein the method comprises the steps of:
converting a compound of formula 3 into the compound of formula 4 through active metal catalysis; 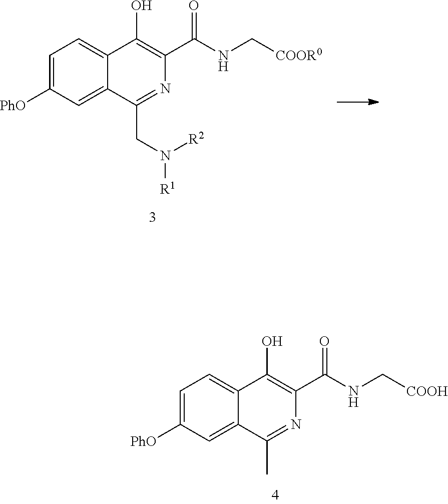
wherein R⁰ is H, C1-C10 alkyl or C6-C10 aryl;
R¹ and R² are independently C1-C10 alkyl, C3-C10 cycloalkyl, C6-C10 aryl, or C6-C10 aryl-C1-C10 alkyl-, or R¹ and R² and the nitrogen atom to which they are attached together form a 3-10 membered heterocycloalkyl, and the 3-10 membered heterocycloalkyl contains 1-2 N atoms and 0-2 heteroatoms selected from O and S.

7. The method according to claim 6, wherein,
when R⁰ is not hydrogen, the method comprises steps 1-2) or steps 1′-2′):
1) reacting the compound of formula 3 with an active metal in acid or in an aqueous solution of acid or in an alcoholic solution of acid to obtain a compound of formula 4′; 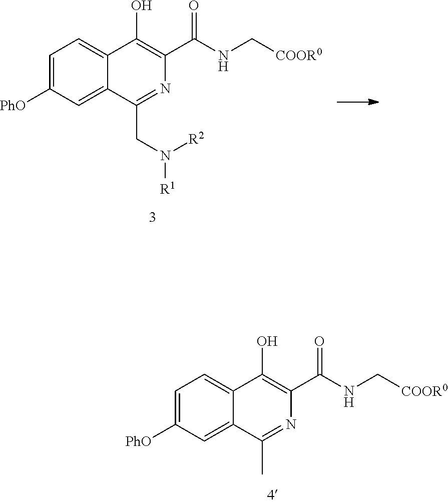
2) reacting the compound of formula 4′ with a base in a first inert solvent to obtain the compound of formula 4; 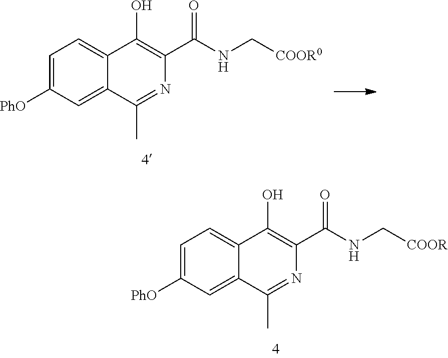
or,
1′) reacting the compound of formula 3 with a base in a second inert solvent to obtain a compound of formula 3′; 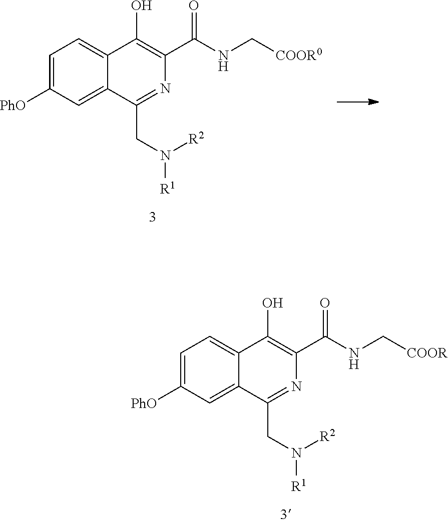
2′) reacting the compound of formula 3′ with an active metal in acid or in an aqueous solution of acid or in an alcoholic solution of acid to obtain the compound of the formula 4; 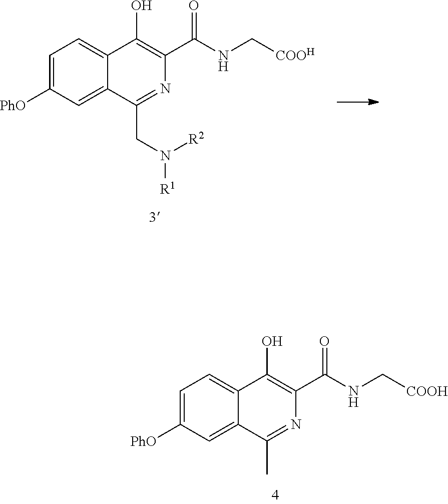
when R⁰ is hydrogen, the method comprises the following steps:
1″) reacting the compound of formula 3 with an active metal in acid or in an aqueous solution of acid or in an alcoholic solution of acid to obtain the compound of formula 4.

8. The method according to claim 7, wherein, the method comprises one or more features selected from the group consisting of:
in the step 1), the step 2′) and/or the step 1″), the acid is inorganic acid or organic acid, wherein the inorganic acid is selected from the group consisting of hydrochloric acid, sulfuric acid, phosphoric acid, and a combination thereof; the organic acid is selected from the group consisting of formic acid, acetic acid, propionic acid, trifluoroacetic acid, trifluoromethanesulfonic acid, and a combination thereof;
in the step 1), the step 2′) and/or the step 1″), the alcohol solvent of the alcoholic solution of acid is selected from the group consisting of methanol, ethanol, isopropanol, n-butanol, ethylene glycol, and a combination thereof;
in the step 1), the step 2′) and/or the step 1″), the active metal is selected from the group consisting of magnesium, aluminum, zinc, iron, and a combination thereof;
in the step 1), the molar ratio of the active metal to the compound of formula 3 is 2-20;
in the step 2′), the molar ratio of the active metal to the compound of formula 3′ is 2-20;
in the step 1″), the molar ratio of the active metal to the compound of formula 3 is 2-20;
in the step 1), the reaction temperature is 60-140° C.;
in the step 1), the reaction time is 0.5-12 h;
in the step 2′), the reaction temperature is 60-140° C.;
in the step 2′), the reaction time is 0.5-12 h;
in the step 1″), the reaction temperature is 60-140° C.;
in the step 2), the first inert solvent is selected from the group consisting of water, methanol, ethanol, isopropanol, n-butanol, ethylene glycol, tetrahydrofuran, 1,4-dioxane, acetonitrile, N,N-dimethylformamide (DMF), N-methylpyrrolidone (NMP), dimethylsulfoxide (DMSO), and a combination thereof;
in the step 1′), the second inert solvent is selected from water, methanol, ethanol, isopropanol, n-butanol, ethylene glycol, tetrahydrofuran, 1,4-dioxane, acetonitrile, N,N-dimethylformamide (DMF), N-methylpyrrolidone (NMP), dimethylsulfoxide (DMSO), and a combination thereof; and/or
in the step 2) and/or step 1′), the base is selected from the group consisting of sodium hydroxide, potassium hydroxide, lithium hydroxide, and a combination thereof.

9. The method according to claim 7, wherein the method comprises one or more features selected from the group consisting of:
in the step 1), the step 2′) or the step 1″), the acid is selected from the group consisting of sulfuric acid, phosphoric acid, trifluoroacetic acid, acetic acid, and a combination thereof;
in the step 1), the step 2′) or the step 1″), the alcohol solvent of the alcoholic solution of acid is isopropanol;
in the step 1), the step 2′) or the step 1″), the molar ratio of the active metal to the compound of formula 3 is 5-15;
in the step 1), the step 2′) or the step 1″), the reaction temperature is 80-130° C.;
in the step 2), the first inert solvent is methanol;
in the step 1′), the second inert solvent is methanol;
in the step 2), the base is sodium hydroxide; and/or
in the step 1′), the base is sodium hydroxide.

10. The method according to claim 6, wherein the compound of formula 3 is prepared by the following method:
a) reacting a compound of formula 1 with glycine or a glycinate to obtain a compound of formula 2; 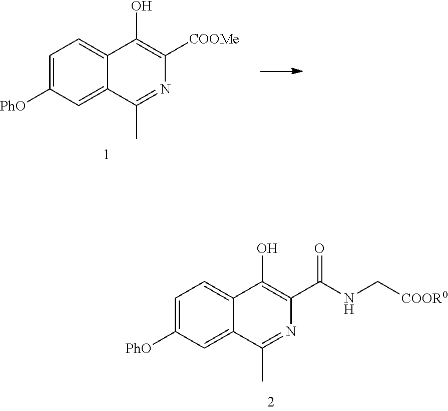
b) reacting the compound of formula 2 with an aminal to obtain the compound of formula 3; 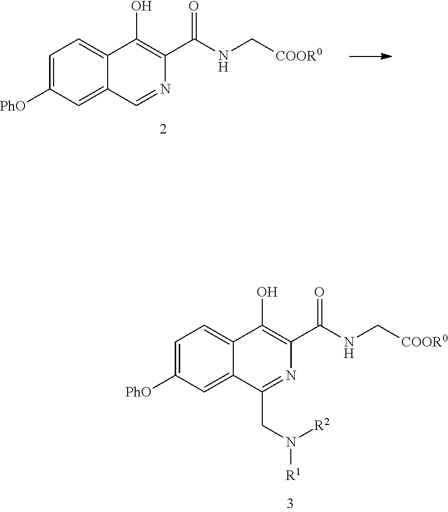
wherein, the glycinate is NH₂—CH₂—C(O)—O—R⁰, and the aminal is (R¹R²)N—CH₂—N(R¹R²);
R⁰ is selected from H, C1-C10 alkyl, and C6-C10 aryl;
R¹ and R² are independently C1-C10 alkyl, C3-C10 cycloalkyl, C6-C10 aryl, or C6-C10 aryl-C1-C10 alkyl-, or R¹ and R² and the nitrogen atom to which they are attached together form a 3-10 membered heterocycloalkyl, and the 3-10 membered heterocycloalkyl contains 1-2 N atoms and 0-2 heteroatoms selected from O and S.

11. A method for preparing a compound, wherein the method comprises step a) or step b):
step a):
reacting a compound of formula 1 with glycine to obtain a compound of formula 2, then reacting the compound of formula 2 with an alcohol and an acyl chloride to obtain the compound of formula 3; 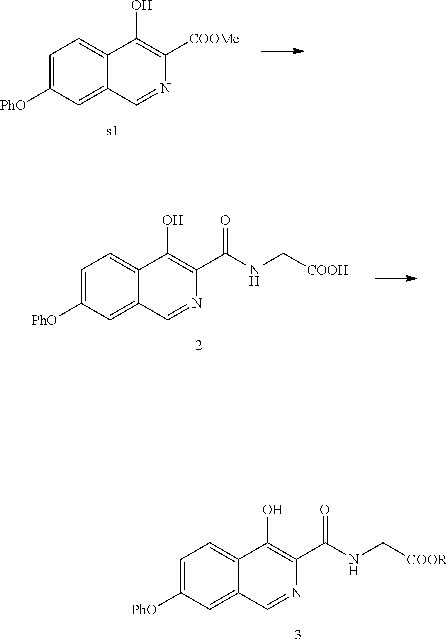
or step b):
reacting the compound of formula 1 with a glycinate to obtain the compound of formula 3; 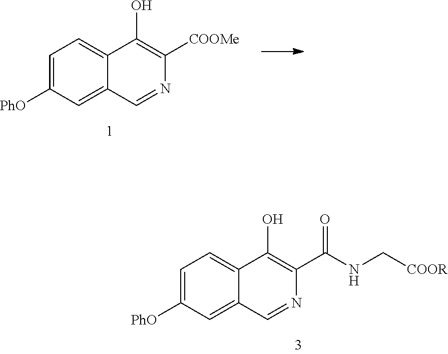
wherein, the acyl chloride is RC(O)Cl, and the glycinate is NH₂—CH₂—C(O)—O—R;
R is C1-C10 alkyl, C6-C10 aryl, C6-C10 aryl-C1-C4 alkyl-, or —R¹OR², wherein R¹ and R² are each independently C1-C10 alkyl.

12. The method according to claim 11, wherein,
the step a) comprises the following steps:
a1) reacting the compound of formula 1 with glycine in a first inert solvent under the action of a first basic reagent to obtain the compound of formula 2;
a2) reacting the compound of formula 2 with an alcohol and an acyl chloride to obtain the compound of formula 3;
the step b) comprises the following steps:
reacting the compound of formula 1 with a glycinate in a second inert solvent under the action of a second basic reagent to obtain the compound of formula 3; and/or
R is methyl, methoxymethyl, ethyl, ethoxyethyl, propyl, isopropyl, n-butyl, isobutyl, tert-butyl, benzyl or phenyl.

13. The method according to claim 12, wherein the method comprises one or more features selected from the group consisting of:
in the step a1), the first inert solvent is selected from the group consisting of ethylene glycol monomethyl ether, methanol, ethanol, isopropanol, n-butanol, N,N-dimethylformamide (DMF), N-methylpyrrolidone (NMP), dimethylsulfoxide (DMSO), acetonitrile, tetrahydrofuran, 1,4-dioxane, ethyl acetate, isopropyl acetate, dichloromethane, and a combination thereof;
in the step a1), the first basic reagent is selected from the group consisting of triethylamine (TEA), 1,8-diazabicycloundec-7-ene (DBU), N,N-diisopropylethylamine (DIEA), N-methylmorpholine, pyridine, and a combination thereof;
in the step a1), the molar ratio of the glycine to the compound of formula 1 is 1-4:1;
in the step a1), the reaction temperature is 50-100° C.;
in the step a1), the reaction time is 2-12 h;
in the step a2), the alcohol is selected from the group consisting of methanol, ethanol, isopropanol, n-butanol, and a combination thereof;
in the step a2), the acyl chloride is selected from the group consisting of thionyl chloride, acetyl chloride, benzoyl chloride, oxalyl chloride, and a combination thereof;
in the step a2), the volume ratio of the alcohol to the compound of formula 2 is 1:1 to 30:1;
in the step a2), the molar ratio of the acyl chloride to the compound of formula 2 is 1-10:1;
in the step a2), the temperatures of all the reactions are the temperature such that the reactions are carried out under reflux conditions;
in the step a2), the time of all the reactions is 2-8 h;
in the step b), the second inert solvent is selected from the group consisting of ethylene glycol monomethyl ether, methanol, ethanol, isopropanol, n-butanol, N,N-dimethylformamide (DMF), N-methylpyrrolidone (NMP), dimethylsulfoxide (DMSO), acetonitrile, tetrahydrofuran, 1,4-dioxane, ethyl acetate, isopropyl acetate, dichloromethane, and a combination thereof;
in the step b), the second basic reagent is selected from the group consisting of triethylamine (TEA), 1,8-diazabicycloundec-7-ene (DBU), N,N-diisopropylethylamine (DIEA), N-methylmorpholine, pyridine, and a combination thereof;
in the step b), the glycinate is selected from the group consisting of methyl glycinate, ethyl glycinate, benzyl glycinate, methoxymethyl glycinate, and a combination thereof;
in the step b), the molar ratio of the glycinate to the compound of formula 1 is 1-4:1;
in the step b), the reaction temperature is 50-100° C.; and/or
in the step b), the reaction time is 2-12 h.

14. The method according to claim 12, wherein the method further comprises the following steps:
1) reacting the compound of formula 3 with a halogenated reagent to obtain a compound of formula 4; 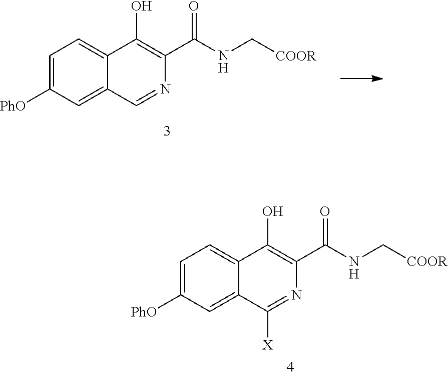
2) reacting the compound of formula 4 with a methylating reagent to obtain the compound of formula 5; 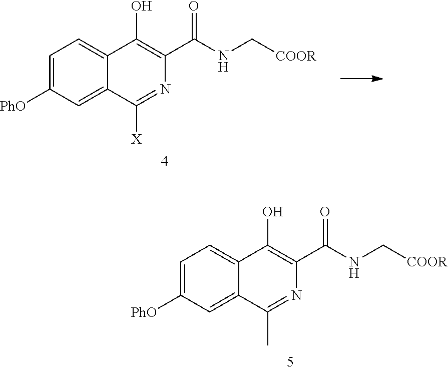
R is C1-C10 alkyl, C6-C10 aryl, C6-C10 aryl-C1-C4 alkyl-, or —R¹OR², wherein R¹ and R² are each independently C1-C10 alkyl, and X is Cl, Br or I.

15. The method according to claim 14, wherein,
in the step 1), the compound of formula 3 is subjected to the halogenation reaction with the halogenated reagent in a third inert solvent to obtain the compound of formula 4;
in the step 2), the compound of formula 4 is reacted with the methylating reagent in a fourth inert solvent in the presence of a third basic reagent and a palladium catalyst to obtain the compound of formula 5; and/or
in the compound of formula 3, R is methyl, methoxymethyl, ethyl, ethoxyethyl, propyl, isopropyl, n-butyl, isobutyl, tert-butyl, benzyl or phenyl.

16. The method according to claim 5, wherein the method comprises one of more features selected from the group consisting of:
in the step a), the second inert solvent is selected from the group consisting of acetonitrile, methanol, ethanol, ethyl acetate, dichloromethane, and a combination thereof;
in the step a), the halogenated reagent is selected from the group consisting of NCS, NBS, NIS, dichlorohydantoin, dibromohydantoin, diiodohydantoin, bromine, iodine, and a combination thereof;
in the step a), the molar ratio of the compound of formula s4 to the halogenated reagent is 1:1-3;
in the step b), the methylating agent is selected from the group consisting of trimethylboron, methylboronic acid, isopropyl methylborate, potassium methyltrifluoroborate, and a combination thereof;
in the step b), the second basic reagent is selected from the group consisting of NaOH, KOH, LiGH, Na₂CO₃, K₂CO₃, Na₃PO4, K₃PO4, and a combination thereof,
in the step b), the palladium catalyst is selected from the group consisting of palladium acetate, bis(triphenylphosphine) palladium dichloride, tetrakis(triphenylphosphine) palladium, tris(benzylideneacetone) dipalladium, bis(diphenylphosphino) ferrocene palladium dichloride, triphenylphosphine palladium dichloride, and a combination thereof, and/or
in the step b), the third inert solvent comprises a mixed solution of ethylene glycol monomethyl ether and water.

17. The method according to claim 15, wherein the method comprises one or more features selected from the group consisting of:
in the step 1), the third inert solvent is selected from the group consisting of methanol, ethanol, isopropanol, dichloromethane, acetonitrile, tetrahydrofuran, and a combination thereof;
in the step 1), the halogenated reagent is selected from the group consisting of NCS, dichlorohydantoin, NBS, dibromohydantoin, bromide, tetrabutylammonium tribromide, pyridinium tribromide, iodine, NIS, diiodohydantoin, and a combination thereof;
in the step 1), the volume ratio of the third inert solvent to the compound of formula 3 is 1:1 to 30:1;
in the step 1), the molar ratio of the halogenated reagent to the compound of formula 3 is 1.0-10:1;
in the step 1), the reaction time is 1-8 h;
in the step 1), the reaction temperature is 0-30° C.;
in the step 2), the fourth inert solvent is selected from the group consisting of water, N,N-dimethylformamide, methanol, ethanol, isopropanol, n-butanol, ethylene glycol, ethylene glycol monomethyl ether, ethylene glycol dimethyl ether, 1,4-dioxane, tetrahydrofuran, and a combination thereof;
in the step 2), the third basic reagent is selected from the group consisting of sodium carbonate, potassium carbonate, potassium acetate, sodium phosphate, potassium phosphate, and a combination thereof;
in the step 2), the palladium catalyst is selected from the group consisting of bis(triphenylphosphorus) palladium dichloride, palladium acetate, triphenylphosphine palladium acetate, tetrakis(triphenylphosphine) palladium, acetylacetonate palladium, [1,1′-bis(diphenylphosphino)ferrocene] palladium dichloride, [1,1′-bis(diphenylphosphino)ferrocene]palladium dichloride dichloromethane complex, and a combination thereof;
in the step 2), the methylating reagent is selected from the group consisting of trimethylboron, methylboronic acid, isopropyl methylborate, potassium methyltrifluoroborate, and a combination thereof;
in the step 2), the volume ratio of the fourth inert solvent to the compound of formula 4 is 1:1-30:1;
in the step 2), the molar ratio of the third basic reagent to the compound of formula 4 is 1-10:1;
in the step 2), the molar ratio of the methylating agent to the compound of formula 4 is 1-10:1;
in the step 2), the reaction temperature is 50° C.-120° C.;
in the step 2), the reaction time is 1-10 h; and/or
in the step 2), the reaction temperature is 80-140° C.
